(19) United States
(12) Patent Application Publication
(10) Pub. No.: US 2024/0142437 A1
(43) Pub. Date: May 2, 2024

(54) METHODS TO PREVENT TERATOGENICITY OF IMID LIKE MOLECULES AND IMID BASED DEGRADERS/PROTACS

(71) Applicant: DANA-FARBER CANCER INSTITUTE, INC., Boston, MA (US)

(72) Inventors: Eric S. Fischer, Chestnut Hill, MA (US); Katherine Donovan, Boston, MA (US)

(73) Assignee: DANA-FARBER CANCER INSTITUTE, INC., Boston, MA (US)

(21) Appl. No.: 18/371,858

(22) Filed: Sep. 22, 2023

Related U.S. Application Data
(63) Continuation of application No. 16/760,658, filed on Apr. 30, 2020.
(60) Provisional application No. 62/672,441, filed on May 16, 2018.
(60) Provisional application No. 62/584,015, filed on Nov. 9, 2017.

(51) Int. Cl. G01N 33/50; G01N 33/68

(52) U.S. Cl. CPC G01N 33/5014; C12Y 603/02019; G01N 33/68; G01N 2500/10; G01N 2500/20

(57) ABSTRACT

Presented are methods of assessing the teratogenicity of agents by measuring the degradation of SALL4, and related compounds with reduced teratogenicity. Provided herein is a method for assessing the teratogenicity of an agent comprising: contacting an agent with SALL4; and measuring levels of SALL4, wherein the agent is teratogenic if SALL4 levels are substantially reduced in the presence of the agent relative to in the absence of the agent.

### RELATED APPLICATIONS

This application claims the benefit under 35 U.S.C. § 119(e) of U.S. Provisional Patent Application No. 62/584,015, filed on Nov. 9, 2017, entitled “METHODS TO PREVENT TERATOGENICITY OF IMID LIKE MOLECULES AND IMID BASED DEGRADERS/PROTACS,” and of U.S. Provisional Patent Application No. 62/672,441, filed on May 16, 2018, entitled “METHODS TO PREVENT TERATOGENICITY OF IMID LIKE MOLECULES AND IMID BASED DEGRADERS/PROTACS,” the entire contents of each of which are incorporated by reference herein.

### FEDERALLY SPONSORED RESEARCH

This invention was made with government support under NCI R01CA214608 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 10, 2024, is named 52095_695C01US_ST.xml and is 98 KB in size.

### BACKGROUND OF INVENTION

Thalidomide (N-a-phthalimidoglutarimide) is known for its potent teratogenic side effects. Thalidomide was first synthesized in Germany in 1954 and was marketed from 1957 worldwide as a non-barbiturate, non-addictive, non-toxic sedative and anti-nausea medication. Thalidomide was withdrawn from the world market in 1961 due to the development of severe congenital abnormalities in babies born to mothers using it for morning sickness.

Thalidomide caused thousands of cases of limb reduction anomalies, including phocomelia (absence of the long bones in the forelimb) or amelia (a complete absence of the forelimb) in the children of pregnant women in the 1950s and 1960s. Other phenotypic malformations were also commonly seen including eye, ear, heart, gastrointestinal and kidney defects. Analogs of thalidomide are also commonly teratogenic.

Thalidomide possesses immunomodulatory, anti-inflammatory and anti-angiogenic properties. The immunomodulatory and anti-inflammatory properties may be related to suppression of excessive tumor necrosis factor-alpha production (Moreira, J Exp Med, 177(6): 1675-80, 1993). Other immunomodulatory and anti-inflammatory properties of thalidomide may include suppression of macrophage involvement in prostaglandin synthesis, and modulation of interleukin-10 and interleukin-12 production by peripheral blood mononuclear cells. The combination of anti-inflammatory and anti-angiogenic properties makes thalidomide a novel therapeutic agent with significant potential in treating a wide variety of diseases (Teo, Clin Pharmacokinet, 43(5): 311-27, 2004). Thalidomide's combined anti-angiogenic and anti-inflammatory properties likely lead to its anti-cancer effects and efficacy in the treatment of multiple myeloma as well as documented activity in other cancers.

Thalidomide-related compounds could harness the immunomodulatory, anti-inflammatory and anti-angiogenic properties of thalidomide while avoiding the teratogenic side effects.

### BRIEF SUMMARY OF INVENTION

It has been surprisingly discovered that the Cullin RING E3 ubiquitin ligase CUL4-RBX1-DDB1-CRBN (CRL4^{CRBN}) targets SALL4 for degradation and that this degradation of SALL4 in the presence of a compound can be used as an indicator of the teratogenicity of the compound. Presented herein are methods for measuring degradation of SALL4 by CRL4^{CRBN} including by measuring levels of SALL4, by visualizing degradation products of SALL4, and by detecting ubiquitination of SALL4. Also presented herein is a modified thalidomide that does not cause degradation of SALL4 by CRL4^{CRBN}.

In one aspect, provided herein is a method for assessing the teratogenicity of an agent comprising:
contacting an agent with SALL4; and
measuring levels of SALL4,
wherein the agent is teratogenic if SALL4 levels are substantially reduced in the presence of the agent relative to in the absence of the agent.

In some embodiments, contacting the agent with SALL4 comprises contacting the agent with a cell expressing SALL4.

In some embodiments, SALL4 levels are visualized by western blot. In some embodiments, SALL4 levels are detected by mass spectrometry.

In some embodiments, SALL4 is fused to a detectable label. In some embodiments, levels of SALL4 are measured optically in the cell.

In another aspect, provided herein is a method for assessing the teratogenicity of an agent comprising:
contacting an agent with SALL4; and
measuring association of SALL4 with CRBN,
wherein the agent is teratogenic if SALL4 substantially associates with CRBN in the presence of the agent relative to in the absence of the agent.

In some embodiments, the association of SALL4 with CRBN is measured in vitro. In some embodiments, the association of SALL4 with CRBN is measured by co-immunoprecipitation. In some embodiments, the association of SALL4 with CRBN is measured by FRET. In some embodiments, the FRET is TR-FRET.

In another aspect, provided herein is a method for assessing the teratogenicity of an agent comprising:
contacting an agent with SALL4; and
measuring ubiquitination of SALL4,
wherein the agent is teratogenic if SALL4 is substantially ubiquitinated in the presence of the agent relative to in the absence of the agent.

In some embodiments, ubiquitination of SALL4 is visualized by western blot. In some embodiments, ubiquitination of SALL4 is measured by mass spectrometry.

In another aspect, provided herein is a method for assessing the teratogenicity of an agent. The method comprises
contacting an agent with SALL4; and
measuring degradation of SALL4,
wherein the agent is teratogenic if SALL4 is substantially degraded in the presence of the agent relative to in the absence of the agent.

In some embodiments, contacting the agent with SALL4 comprises contacting the agent with a cell expressing SALL4.

In some embodiments, measuring degradation of SALL4 comprises detecting SALL4 degradation products. In some embodiments, SALL4 degradation products are detected by western blot. In some embodiments, SALL4 degradation products are detected by mass spectrometry.

In some embodiments, the agent is a cancer therapy. In some embodiments, the agent is an IMiD. In some embodiments, the agent is a degrader. In some embodiments, the degrader is a degronomid. In some embodiments, the agent is a pesticide.

In another aspect, provided herein is a modified thalidomide, wherein the modified thalidomide does not cause substantial reduction of SALL4 levels, substantial degradation of SALL4, substantial association of SALL4 with CRBN, or substantial ubiquitination of SALL4 when contacted with SALL4 as compared to a thalidomide without the modification.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-ID: Identification of SALL4 as an IMiD-dependent CRL4^{CRBN} substrate. FIGS. 1A-1C: Scatter plots depicting the identification of IMiD-dependent substrate candidates. H9 human embryonic stem cells (hESC) were treated with 10 μM thalidomide (FIG. 1A), 5 μM lenalidomide (FIG. 1B), 1 μM pomalidomide (FIG. 1C) or DMSO control and protein abundance was analyzed using TMT quantification mass spectrometry (see methods for details). Significant changes were assessed by a moderated t-test as implemented in the limma package (Ritchie et al., 2015) and the log 2 fold change (log 2 FC) is shown on the y-axis, and negative log₁₀ P Values on the x-axis (two independent biological replicates for each of the IMiDs, or three independent biological replicates for DMSO). FIG. 1D: Heatmap displaying the mean log 2 FC of the identified IMiD-dependent targets comparing treatment with thalidomide, lenalidomide and pomalidomide. Mean log 2 FC values were derived from averaging across proteomics experiments in four different cell lines (hESC, MM1s, Kelly, SK-N-DZ). The heatmap colors are scaled with blue indicating a decrease in protein abundance (−2 log 2 FC) and red indicating no change (0 log 2 FC) in protein abundance. Targets newly identified in this study are marked with a green dot, ZnF containing targets with a cyan dot, and previously characterized targets with a grey dot. Substrates are grouped according to their apparent IMiD selectivity in the mass spectrometry-based proteomics. It should be noted, that this does not refer to absolute selectivity but rather relative selectivity.

FIGS. 2A-2F: Validation of SALL4 as bonafide IMiD-dependent CRL4^{CRBN} substrate. FIG. 2A: H9 hESC were treated with increasing concentrations of thalidomide, lenalidomide, pomalidomide or DMSO as a control. Following 24 hours of incubation, SALL4 and GAPDH protein levels were assessed by western blot analysis. FIG. 2B: As in FIG. 2A, but treatment was done in Kelly cells. FIG. 2C: Kelly cells were treated with increasing concentrations of thalidomide and co-treated with 5 μM bortezomib, 5 μM MLN4924, 0.5 μM MLN7243, or DMSO as a control. Following 24 hours incubation, SALL4 and GAPDH protein levels were assessed by western blot analysis. FIG. 2D: Parental Kelly cells or two independent pools of CRBN^{-/-} Kelly cells were treated with increasing concentrations of thalidomide. Following 24 hours incubation, SALL4, CRBN, and GAPDH protein levels were assessed by western blot analysis. FIG. 2E: Kelly cells were treated with 5 μM pomalidomide or DMSO as a control for 8 hours, at which point the compound was washed out. Cells were harvested at 1, 2, 4, 8, 24 and 48 hours post-washout and SALL4 and GAPDH protein levels were assessed by western blot analysis. FIG. 2F: Kelly cells were treated with 5 μM pomalidomide for 1, 2, 4, 8 and 24 hours, or with DMSO as a control. Following time course treatment, SALL4 and GAPDH protein levels were assessed by western blot analysis. Shown is one representative experiment out of three replicates for each of the western blots in this figure.

FIGS. 3A-3I: SALL4 ZnF2 is the zinc finger responsible for IMiD-dependent binding to CRL4^{CRBN}. FIG. 3A: Multiple sequence alignment of the validated ‘degrons’ from known IMiD-dependent zinc finger substrates, along with the two candidate zinc finger degrons from SALL4. FIG. 3B: TR-FRET: Titration of IMiD (thalidomide, lenalidomide and pomalidomide) to DDB1ΔB-CRBN_{Spy-BodipyFL} at 200 nM, hsSALL4_{ZnF2} at 100 nM, and Terbium-Streptavidin at 4 nM. FIG. 3C: As in B, but with hsSALL4_{ZnF4} with DDB1ΔB-CRBN_{Spy-BodipyFL} at 1 μM. FIG. 3D: TR-FRET: Titration of DDB1ΔB-CRBN_{Spy-BodipyFL} to biotinylated hsSALL4_{ZnF2}, hsSALL4_{ZnF1-2} or hsSALL4_{ZnF4} at 100 nM and Terbium-Streptavidin at 4 nM in the presence of 50 μM thalidomide. FIG. 3E: As in FIG. 3B, but with hsSALL4_{ZnF1-2}. FIG. 3F: As in FIG. 3B, but with hsSALL4_{ZnF2} and hsSALL4_{ZnF2}^{G416A} mutant as thalidomide titration. FIG. 3G: Kelly cells transiently transfected with Flag-hsSALL4^{WT}, Flag-hsSALL4^{G600A} or hsSALL4^{G600N} were treated with increasing concentrations of thalidomide or DMSO as a control. Following 24 hours of incubation, SALL4 (α-Flag) and GAPDH protein levels were assessed by western blot analysis (shown is one representative experiment out of three replicates. FIG. 3H: As in FIG. 3G, but with Flag-hsSALL4^{WT}, Flag-hsSALL4^{G416A} or Flag-hsSALL4^{G416N}. FIG. 3I: In vitro ubiquitination of biotinylated hsSALL4_{ZnF1-2} by CRL4^{CRBN} in the presence of thalidomide (10 μM), lenalidomide (10 μM) and pomalidomide (0.1, 1 and 10 μM) or DMSO as a control.

FIGS. 4A-4I: Identification of the sequence differences in the IMiD-dependent binding region of both CRBN and SALL4 in specific species. FIG. 4A: Close-up view on the beta-hairpin loop region of Ck1a (CSNK1A1) interacting with CRBN and lenalidomide (PDB: 5fqd) highlighting the additional bulkiness of the V388I mutation (PDB: 4ci1) present in mouse and rat CRBN. CSNK1A1 and lenalidomide are depicted as stick representation in magenta and yellow, respectively, the Ile391 of mouse CRBN corresponding to human Val388 is depicted as stick representation in cyan, and CRBN is depicted as surface representation. FIG. 4B: TR-FRET: Titration of DDB1ΔB-hsCRBN_{Spy-BodipyFL}, or DDB1ΔB-hsCRBN^{V388I}_{Spy-BodipyFL} to biotinylated hsSALL4_{ZnF1-2} at 100 nM and Terbium-Streptavidin at 4 nM in the presence of 50 μM pomalidomide or DMSO. FIG. 4C: mES cells were treated with increasing concentrations of thalidomide and pomalidomide or DMSO as a control. Following 24 hours of incubation, SALL4 and GAPDH protein levels were assessed by western blot analysis. FIG. 4D: mES cells constitutively expressing Flag-hsCRBN were treated with increasing concentrations of thalidomide. Following 24 hours of incubation, ZFP91 and GAPDH protein levels were assessed by western blot analysis. FIG. 4E: As in FIG. 4C, but measuring GZF1 and GAPDH protein levels. FIG. 4F: As in FIG. 4C, but measuring SALL4, hsCRBN (α-Flag) and GAPDH protein levels. FIG. 4G: Kelly cells were transiently transfected with Flag-hsSALL4, Flag-mmSALL4 or Flag-mmSALL4 containing a humanized ZnF2 (Y415F, P418S, I419V, L430F, Q435H) and treated with increasing concentrations of thalidomide. Following 24 hours of incubation, hsSALL4, mmSALL4, humanized mmSALL4 (α-Flag) and GAPDH protein levels were assessed by western blot analysis. FIG. 4H: TR-FRET. Titration of thalidomide to DDB1ΔB-CRBN_{Spy-BodipyFL} at 200 nM, hsSALL4_{ZnF2}, mmSALL4_{ZnF2}, or drSALL4_{ZnF2} all at 100 nM and Terbium-Streptavidin at 4 nM. Data is presented as means±s.d. (n=3). FIG. 4I: As in FIG. 4G, but with Flag-drSALL4.

FIGS. 5A-5D: Sequence differences in the IMiD-dependent binding region of both CRBN and SALL4 interfere with ternary complex formation in specific species. FIG. 5A: A multiple sequence alignment of the region of CRBN critical for IMiD mediated ZnF binding from human, bush baby, mouse, rat, macaque, marmoset, and rabbit is shown highlighting the V388I polymorphism. FIG. 5B: A multiple sequence alignment of SALL4_{ZnF2} from human, macaque, marmoset, bush baby, rabbit, mouse, rat, zebrafish and chicken, highlighting the differences in sequence across species. FIG. 5C: Schematic summary of species-specific effects of IMiD treatment on ZnF degradation and relationship to thalidomide syndrome phenotype. Top panel depicts sensitive species: hsCRBN^{V388} is capable of IMiD-dependent binding, ubiquitination and subsequent degradation of hsSALL4 and hsZnF targets, and the thalidomide embryopathy is observed. Middle panel depicts insensitive species: mmCRBN^{I391} is capable of binding IMiDs, but not binding mmSALL4 and mmZnF targets, and no embryopathy is observed. Bottom panel depicts humanizing CRBN as ineffective for inducing the phenotype: hsCRBN^{V388} is capable of IMiD-dependent ubiquitination and subsequent degradation of mmZnF proteins, but not mmSALL4, and the embryopathy is not observed. This data is consistent with a ‘double protection’ mechanism caused by mutations in both CRBN and SALL4 preventing IMiD-dependent binding and subsequent degradation in insensitive species. FIG. 5D: Heatmap comparing the sequence conservation of IMiD-dependent targets across 30 different species. High conservation is displayed as blue and low conservation is displayed as white.

FIGS. 6A-6C: Mass spectrometry profiling of IMiDs. FIG. 6A: Schematic representation of the mass spectrometry-based proteomics workflow used for IMiD profiling. FIG. 6B: Chemical structures of compounds used in this study. FIG. 6C: Scatter plots depicting the identification of treatment-dependent substrate candidates. Kelly cells were treated with 10 μM thalidomide (3× biological replicates), 5 μM lenalidomide (3× biological replicates), 1 μM pomalidomide or DMSO as a control (3× biological replicates) for 5 hours (top row). MM1s cells were treated with 10 μM thalidomide (2× biological replicates), 5 μM lenalidomide (2× biological replicates), 1 μM pomalidomide (2× biological replicates) or DMSO as a control (3× biological replicates) for 5 hours (middle row). SK-N-DZ cells were treated with 0.1 μM CC-220, 1 μM dBET57, 1 μM Pomalidomide (3× biological replicates) or DMSO as a control (3× biological replicates) for 5 hours (bottom row). Protein abundance from each experiment was analyzed using TMT quantification mass spectrometry (see methods for details). Significant changes were assessed by a moderated t-test as implemented in the limma package (Ritchie et al., 2015) and the log 2 fold change is shown on the y-axis, and negative log₁₀ P Values on the x-axis (the number of replicates is indicated above). Hits that met the set significance threshold (fold-change greater 1.5 and log 10 P value below 0.001) are displayed with a red point (•).

FIGS. 7A-7E: Extended validation of IMiD-dependent targets. FIG. 7A: Heatmap summarizing the protein abundance of IMiD-dependent targets identified from proteomics data across four different cell lines (Kelly, MM1s, hES and SK-N-DZ cells) and five different compounds (thalidomide, lenalidomide, pomalidomide, CC-220 and dBET57). The color scale displays a 2.5 fold decrease in protein abundance in blue and no change is displayed in white. NA indicates the protein was not identified/quantified in the experiment. FIG. 7B: Mass spectrometry scatter plot validation of IMiD-dependent targets. SK-N-DZ cells were treated with 1 μM pomalidomide to induce degradation of IMiD-dependent targets (left), degradation was rescued by co-treatment with 1 μM pomalidomide+5 μM MLN4924 (right), or treated with DMSO as a control for 5 hours. Protein abundance from each experiment was analyzed using TMT quantification mass spectrometry (see methods for details). Significant changes were assessed by a moderated t-test as implemented in the limma package (Ritchie et al., 2015) and the log 2 FC is shown on the y-axis, and −log₁₀ P Values on the x-axis (for three biological replicates). Hits that met the significance threshold (fold-change greater 1.5 and log₁₀ P value below 0.001) are displayed with a point (•) next to the gene name indicated. FIG. 7C: Reporter ion ratios from FIG. 7B were normalized and scaled (see methods) and are depicted as a bar graph for the IMiD-dependent targets. Co-treatment with the neddylation inhibitor MLN4924 abrogated the degradation of all IMiD-dependent targets in accordance with a Cullin-RING ligase dependent mechanism of degradation. Data is presented as means±s.d. (n=3 biological replicates). FIG. 7D: Western blot validation of MLN4924 rescue experiment: SK-N-DZ or Kelly cells were treated with increasing concentrations of thalidomide or DMSO as a control. Following 24 hours incubation, GZF1 (left) and DTWD1 (right) as well as GAPDH protein levels were assessed by western blot analysis (shown is one representative out of three replicates). FIG. 7E: Multiple sequence alignment of the CxxCG containing zinc finger domains from each of the IMiD-dependent targets identified by mass spectrometry in this study.

FIGS. 8A-8K: Extended validation of SALL4. FIG. 8A: HEK293T cells were treated with increasing concentrations of thalidomide, lenalidomide, pomalidomide or DMSO as a control. Following 24 hours incubation, SALL4 and GAPDH protein levels were assessed by western blot analysis. FIG. 8B: As in FIG. 8A, but with H661 cells. FIG. 8C: As in FIG. 8A, but with SK-N-DZ cells. FIG. 8D: HEK293T cells were treated with increasing concentrations of thalidomide and co-treated with 5 μM bortezomib, 5 μM MLN4924, 0.5 μM MLN7243, or DMSO as a control. Following 24 hours incubation, SALL4 and GAPDH protein levels were assessed by western blot analysis. FIG. 8E: As in FIG. 8D, but with SK-N-DZ cells. FIG. 8F: Parental HEK293T cells or two independent pools of CRBN^{-/-} HEK293T cells were treated with increasing concentrations of thalidomide. Following 24 hours incubation, SALL4, CRBN, and GAPDH protein levels were assessed by western blot analysis. FIG. 8G: Kelly cells were treated with 1 μM pomalidomide or DMSO as a control for 8 hours, at which point the compound was washed out. Cells were harvested at 1, 2, 4, 8, 24 and 48 hours post-washout and SALL4 and GAPDH protein levels were assessed by western blot analysis. FIG. 8H: Kelly cells were treated with 1 μM pomalidomide for 1, 2, 4, 8 and 24 hours, or with DMSO as a control. Following time course treatment, SALL4 and GAPDH protein levels were assessed by western blot analysis. FIG. 8I: Thalidomide treatment did not influence the expression of SALL4 mRNA. hES cells treated with 10 μM thalidomide or DMSO as a control for 24 hours were subjected to quantitative RT-PCR to assess the levels of total SALL4 mRNA. The mRNA levels were normalized to those of GAPDH (housekeeping gene) mRNA. The SALL4 mRNA level remained stable or increased which is in contrast to the decrease in protein abundance observed in proteomics and western blot analysis. mRNA fold change was determined from n=2 with three technical replicates. FIG. 8J: To validate the specificity of the antibody used, Kelly or HEK293T cells were transfected with a plasmid expressing mCherry, Cas9, and one of three guide RNAs (sgRNA1, sgRNA2, sgRNA3) targeting the SALL4 gene, or a mock control. Following 48 hours incubation, SALL4 and GAPDH protein levels were assessed by western blot analysis and for sgRNA1 and sgRNA2 a loss of the specific bands for SALL4 were observed in accordance with the antibody being specific for SALL4. sgRNA3 had no effect, which is likely due to an ineffective sgRNA. FIG. 8K: To validate the specificity of the antibody used, Kelly cells were transfected with a plasmid overexpressing Flag-mmSALL4, Flag-hsSALL4, or no transfection. Following 48 hours incubation, SALL4 and GAPDH protein levels were assessed by western blot analysis. Shown is one representative experiment out of three replicates for each of the western blots in this figure.

FIGS. 9A-9L: Biochemical characterization of SALL4 binding to CRBN. FIG. 9A: TR-FRET. Titration of DDB1ΔB-CRBN_{Spy-BodipyFL} to biotinylated hsSALL4_{ZnF2}, hsSALL4_{ZnF1-2} and hsSALL4_{ZnF4} at 100 nM and Terbium-Streptavidin at 4 nM in the presence of lenalidomide at 50 μM. FIG. 9B: As in FIG. 9A, but in the presence of pomalidomide at 50 μM. FIG. 9C: TR-FRET: Titration of lenalidomide to DDB1ΔB-CRBN_{Spy-BodipyFL} at 200 nM, hsSALL4_{ZnF2}^{WT}, hsSALL4_{ZnF2}^{G416A} at 100 nM, and Terbium-Streptavidin at 4 nM. FIG. 9D: As in FIG. 9C, but titrating with pomalidomide. FIG. 9E: TR-FRET: Titration of thalidomide to DDB1ΔB-CRBN_{Spy-BodipyFL} at 1 μM, hsSALL4_{ZnF4} or hsSALL4_{ZnF4}^{Q595H} mutant at 100 nM, and Terbium-Streptavidin at 4 nM. FIG. 9F: TR-FRET: Titration of thalidomide to DDB1ΔB-CRBN_{Spy-BodipyFL} at 200 nM, hsSALL4_{ZnF1-2}^{WT}, hsSALL4_{ZnF1-2}^{G416N} and hsSALL4_{ZnF1-2}^{S388N} at 100 nM, and Terbium-Streptavidin at 4 nM. FIG. 9G: As in FIG. 9F, but titrating with lenalidomide. FIG. 9H: As in FIG. 9F, but titrating with pomalidomide. FIG. 9I: TR-FRET. Titration of DDB1ΔB-CRBN_{Spy-BodipyFL} to biotinylated hsSALL4_{ZnF1-2}^{WT}, hsSALL4_{ZnF1-2}^{G416N} and hsSALL4_{ZnF1-2}^{S388N} at 100 nM and Terbium-Streptavidin at 4 nM in the presence of thalidomide at 50 μM. FIG. 9J: TR-FRET. Titration of DDB1ΔB-mmCRBN_{Spy-BodipyFL} to biotinylated hsSALL4_{ZnF2}, hsSALL4_{ZnF1-2} and IKZF1Δ (Petzold et al., 2016) at 100 nM and Terbium-Streptavidin at 4 nM in the presence of thalidomide at 50 μM. FIG. 9K: TR-FRET: Titration of lenalidomide to DDB1ΔB-CRBN_{Spy-BodipyFL} at 200 nM, hsSALL4_{ZnF2}, mmSALL4 ZnF2 and drSALL4_{ZnF2} at 100 nM, and Terbium-Streptavidin at 4 nM. FIG. 9L: As in FIG. 9K, but titrating pomalidomide. All TR-FRET data in this figure are presented as means±s.d. (n=3).

FIGS. 10A-10E: Species specific effects. FIG. 10A: mES cells were treated with increasing doses up to 100 μM of thalidomide. Following 24 hours incubation, SALL4 and GAPDH protein levels were assessed by western blot analysis. FIG. 10B: Kelly cells were transiently transfected with Flag-hsSALL4 and treated with increasing concentrations of thalidomide. Following 24 hours of incubation, SALL4 and GAPDH protein levels were assessed by western blot analysis. FIG. 10C: As in FIG. 10B, but with Flag-mmSALL4. FIG. 10D: Kelly cells were transiently transfected with Flag-mmSALL4 and treated with increasing doses of CC-885, with no transfection as a negative control. Following 24 hours incubation, mmSALL4 (α-Flag) protein levels were assessed by western blot analysis. Shown is one representative out of three replicates for each western blot. FIG. 10E: Gene expression profiles for IMiD-dependent substrates were derived from the genotype-tissue expression (GTex) dataset and are presented as a heatmap.

FIG. 11: Mass spectrometry profiling of DFCI1.

FIG. 12: Mass spectrometry profiling of DFCI2.

### DETAILED DESCRIPTION OF INVENTION

It has been surprisingly discovered that the Cullin RING E3 ubiquitin ligase CUL4-RBX1-DDB1-CRBN (CRL4^{CRBN}) targets SALL4 for degradation and that this degradation of SALL4 in the presence of a compound can be used as an indicator of the teratogenicity of the compound. For example, thalidomide, a teratogenic compound, binds to CRL4^{CRBN} and promotes ubiquitination and degradation of key hematopoietic transcription factors IKZF1/3 and other therapeutic targets such as Ck1a via an induced association mechanism. As is shown in Example 1, thalidomide and other teratogenic compounds, e.g., lenalidomide and pomalidomide, all induce degradation of SALL4 by CRL4^{CRBN} and SALL4 is a direct target of the (CRL4^{CRBN})-thalidomide complex. The involvement of SALL4 in teratogenicity is demonstrated by the role of SALL4 in diseases such as Duane Radial Ray and Holt-Oram syndromes, in which heterozygous loss of function (LOF) mutations in SALL4 mirrors teratogenicity caused by thalidomide.

Degradation of SALL4 by CRL4^{CRBN} can be assayed in a variety of ways including by measuring levels of SALL4, by visualizing degradation products of SALL4, and by detecting ubiquitination of SALL4.

SALL4

Spalt-Like Transcription Factor 4 (SALL4) plays an essential role in developmental events and the maintenance of stem cell pluripotency. SALL4 is a zinc finger transcription factor, that forms a core transcriptional network with POU5FI (Oct4), Nanog and Sox2, which activates genes related to proliferation in embryonic stem cells (ESCs). SALL4 binds to retinoblastoma binding protein 4 (RBBp4), a subunit of the nucleosome remodeling and histone deacetylation (NuRD) complex and the SALL4 bound complex is recruited to various downstream targets including transcription factors. Beside the NuRD complex, SALL4 is also reported to bind to other epigenetic modifiers, altering gene expression. The binding of SALL4 to NuRD complex allows SALL4 to act as a transcriptional repressor for various downstream targets. An example of such downstream target includes, but is not limited to Phosphatase and Tensin homolog (PTEN), a factor that is essential for the self-renewal of leukemic stem cells (LSCs). Diseases associated with SALL4 include Duane-Radial Ray Syndrome and Ivic Syndrome.

As is used herein, “SALL4” refers to the protein encoding sal-like protein 4 and having a human zinc-finger 2 domain (i.e., amino acids 378-438 of SEQ ID NO. 1), or a fragment thereof. In some embodiments, “SALL4” refers to the protein encoding human sal-like protein 4 isoform 1 or 2, or fragments thereof mRNA sequences of human SALL4 include, but are not limited to NCBI: NG 008000.1, NCBI: XP_011527223.1, and XP_011527224.1. Amino acid sequences of human SALL4 include, but are not limited to NCBI: XP_011527223.1, XP_011527224.1, and XP_005260524.1. Isoform 1 of SALL4 is a protein of 1053 amino acids with an apparent molecular weight of ˜112 kDa. In some embodiments, the nucleic acid sequence of SALL4 isoform 1 mRNA is NM_020436.4. In some embodiments, the amino acid sequence of SALL4 isoform 1 is NP_065169.1. Isoform 2 of SALL4 is a protein of 616 amino acids. In some embodiments, the nucleic acid sequence of SALL4 isoform 1 mRNA is NM_001318031.1. In some embodiments, the amino acid sequence of SALL4 isoform 2 is NP_001304960.1. In some embodiments, the amino acid sequence of SALL4 is:

(SEQ ID NO: 1)
MSRRKQAKPQHINSEEDQGEQQPQQQTPEFADAAPAAPAAGELGA
PVNHPGNDEVASEDEATVKRLRREETHVCEKCCAEFFSISEFLEH
KKNCTKNPPVLIMNDSEGPVPSEDFSGAVLSHQPTSPGSKDCHRE
NGGSSEDMKEKPDAESVVYLKTETALPPTPQDISYLAKGKVANTN
VTLQALRGTKVAVNQRSADALPAPVPGANSIPWVLEQILCLQQQQ
LQQIQLTEQIRIQVNMWASHALHSSGAGADTLKTLGSHMSQQVSA
AVALLSQKAGSQGLSLDALKQAKLPHANIPSATSSLSPGLAPFTL
KPDGTRVLPNVMSRLPSALLPQAPGSVLFQSPFSTVALDTSKKGK
GKPPNISAVDVKPKDEAALYKHKCKYCSKVFGTDSSLQIHLRSHT
GERPFVCSVCGHRFTTKGNLKVHFHRHPQVKANPQLFAEFQDKVA
AGNGIPYALSVPDPIDEPSLSLDSKPVLVTTSVGLPQNLSSGTNP
KDLTGGSLPGDLQPGPSPESEGGPTLPGVGPNYNSPRAGGFQGSG
TPEPGSETLKLQQLVENIDKATTDPNECLICHRVLSCQSSLKMHY
RTHTGERPFQCKICGRAFSTKGNLKTHLGVHRTNTSIKTQHSCPI
CQKKFTNAVMLQQHIRMHMGGQIPNTPLPENPCDFTGSEPMTVGE
NGSTGAICHDDVIESIDVEEVSSQEAPSSSSKVPTPLPSIHSASP
TLGFAMMASLDAPGKVGPAPFNLQRQGSRENGSVESDGLTNDSSS
LMGDQEYQSRSPDILETTSFQALSPANSQAESIKSKSPDAGSKAE
SSENSRTEMEGRSSLPSTFIRAPPTYVKVEVPGTFVGPSTLSPGM
TPLLAAQPRRQAKQHGCTRCGKNFSSASALQIHERTHTGEKPFVC
NICGRAFTTKGNLKVHYMTHGANNNSARRGRKLAIENTMALLGTD
GKRVSEIFPKEILAPSVNVDPVVWNQYTSMLNGGLAVKTNEISVI
QSGGVPTLPVSLGATSVVNNATVSKMDGSQSGISADVEKPSATDG
VPKHQFPHFLEENKIAVS

In some embodiments, SALL4 used in the assays described herein is native human SALL4 expressed from its genomic locus under its native promoter. In some embodiments, the native SALL4 is SALL4 isoform 1. In some embodiments, the native SALL4 is SALL4 isoform 2. In some embodiments, the native SALL4 is a mixture of SALL4 isoforms 1 and 2. In some embodiments, the native SALL4 comprises a degradation product of SALL4.

In some embodiments, SALL4 is recombinant SALL4. In some embodiments, SALL4 is recombinant human SALL4. In some embodiments, SALL4 is recombinant SALL4 from a species other than human, e.g., macaque, marmoset, bush baby, mouse, rat, rabbit, chicken, or zebrafish, in which the zinc finger two domain has the sequence of the human zinc finger two domain (i.e., amino acids 378-438 of SEQ ID NO. 1). The nucleic acid sequences coding for SALL4 can be obtained using recombinant methods known in the art, such as, for example by screening libraries from cells expressing the gene, by deriving the gene from a vector known to include the same, or by isolating directly from cells and tissues containing the same, using standard techniques. Recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference).

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the SALL4, e.g., the native or recombinant human SALL4, has 80, 81, 82, 83, 84, 85, 86, 87, 88, 89, 90, 91, 92, 93, 94, 95, 96, 97, 98, or 99% identity to SEQ ID NO: 1.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the SALL4, e.g., the native or recombinant human SALL4, is truncated at the N-terminus by 1-100 amino acids. In some embodiments, SALL4 used in the assays described herein is truncated at the N-terminus by 1, 5, 10, 20, 30, 40, 50, 60, 70, 80, 90, or 100 amino acids. In some embodiments, a protein having the sequence of SEQ ID NO. 1 is truncated at the N-terminus by 1, 5, 10, 20, 30, 40, 50, 60, 70, 80, 90, or 100 amino acids.

In some embodiments, the SALL4, e.g., the native or recombinant human SALL4, is truncated at the C-terminus by 1-100 amino acids. In some embodiments, SALL4 used in the assays described herein is truncated at the C-terminus by 1, 5, 10, 20, 30, 40, 50, 60, 70, 80, 90, or 100 amino acids. In some embodiments, a protein having the sequence of SEQ ID NO. 1 is truncated at the C-terminus by 1, 5, 10, 20, 30, 40, 50, 60, 70, 80, 90, or 100 amino acids.

In some embodiments, the SALL4, e.g., the native or recombinant human SALL4, is truncated at the N-terminus and C-terminus by 1-100 amino acids. In some embodiments, SALL4 used in the assays described herein is truncated at the N-terminus and C-terminus by 1, 5, 10, 20, 30, 40, 50, 60, 70, 80, 90, or 100 amino acids. In some embodiments, a protein having the sequence of SEQ ID NO. 1 is truncated at the N-terminus and C-terminus by 1, 5, 10, 20, 30, 40, 50, 60, 70, 80, 90, or 100 amino acids.

In some embodiments, the SALL4 used in the methods described herein comprises or consists of a fragment of SALL4. In some embodiments, the SALL4 used in the methods described herein comprises or consists of a fragment of recombinant human SALL4 of 10-100 consecutive amino acids of SEQ ID NO. 1, e.g., 10, 15, 20, 25, 30, 35, 40, 45, 50, 60, 70, 80, 90, or 100 consecutive amino acids of SEQ ID NO. 1. In some embodiments, the fragment comprises amino acid residues 300-500 of SEQ ID NO. 1. In some embodiments, the fragment comprises amino acid residues 350-450 of SEQ ID NO. 1. In some embodiments, the fragment comprises amino acid residues 370-440 of SEQ ID NO. 1. In some embodiments, the fragment comprises amino acid residues amino acid residues 378-438 of SEQ ID NO. 1. In some embodiments, the fragment comprises amino acid residues 400-440 of SEQ ID NO. 1. In some embodiments, the fragment comprises amino acid residues 410-433 or 402-436 of SEQ ID NO. 1. In some embodiments, the fragment comprises amino acid residues 500-700 of SEQ ID NO. 1. In some embodiments, the fragment comprises amino acid residues 550-650 of SEQ ID NO. 1. In some embodiments, the fragment comprises amino acid residues 594-616, 583-617, or 590-618 of SEQ ID NO. 1.

In some embodiments, the SALL4 is recombinant human SALL4 or a fragment thereof and comprises 1-10 amino acid substitutions, e.g., 2, 3, 4, 5, 6, 7, 8, 9, or 10 amino acid substitutions. In some embodiments, the SALL4 has a mutation at Q595. In some embodiments, the SALL4 has a mutation at S388 of SEQ ID NO. 1, e.g., a S388N mutation. In some embodiments, the SALL4 has a mutation at G416 of SEQ ID NO. 1, e.g., a G416N or G416A mutation. In some embodiments, the SALL4 has a mutation at G600 of SEQ ID NO. 1, e.g., a G600A or G600N mutation

In some embodiments, SALL4 or fragment thereof used in the assays described herein is tagged. Examples of tags are well known in the art and include, e.g., HIS tags, biotin tags, streptavidin tags, spycatcher tags, Flag tags, and GST tags. In some embodiments, SALL4 used in the assays described herein is tagged with streptavidin. In some embodiments, SALL4 used in the assays described herein is tagged with BirA or SmBiT.

CRBN

As is used herein, “Cereblon” (CRBN) refers to the protein encoding human CBRN or fragments thereof. Human CBRN (isoform 1) is a protein of 442 amino acids with an apparent molecular weight of ˜51 kDa (GenBank: AAH17419). (For additional information related to the CRBN structure see Hartmann et al., PLoS One. 2015, 10, e0128342.) Human CRBN contains the N-terminal part (237-amino acids from 81 to 317) of ATP-dependent Lon protease domain without the conserved Walker A and Walker B motifs, 11 casein kinase II phosphorylation sites, 4 protein kinase C phosphorylation sites, 1 N-linked glycosylation site, and 2 myristoylation sites. CRBN is widely expressed in testis, spleen, prostate, liver, pancreas, placenta, kidney, lung, skeletal muscle, ovary, small intestine, peripheral blood leukocyte, colon, brain, and retina. CRBN is located in the cytoplasm, nucleus, and peripheral membrane. (Chang et al., Int. J. Biochem. Mol. Biol. 2011, 2, 287-94.)

Cereblon is an E3 ubiquitin ligase, and it forms an E3 ubiquitin ligase complex with damaged DNA binding protein 1 (DDBT), Cullin-4A (CUL4A), and regulator of cullins 1 (ROC1). This complex ubiquitinates a number of other proteins. Through a mechanism which has not been completely elucidated, Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

In some embodiments, “CRBN” refers to the protein encoding human CRBN isoform 1 or 2, or fragments thereof. In some embodiments, the nucleic acid sequence of CRBN isoform 1 mRNA is NM_016302.3. In some embodiments, the amino acid sequence of CRBN isoform 1 is NP_057386.2. Isoform 2 of CRBN is a protein of 441 amino acids. In some embodiments, the nucleic acid sequence of CRBN isoform 1 mRNA is NM_001173482.1. In some embodiments, the amino acid sequence of CRBN isoform 2 is NP_001166953.1.

In some embodiments, the amino acid sequence of CRBN is:

(SEQ ID NO: 2)
MAGEGDQQDAAHNMGNHLPLLPAESEEEDEMEVEDQDSKEAKK
PNIINFDTSLPTSHTYLGADMEEFHGRTLHDDDSCQVIPVLPQ
VMMILIPGQTLPLQLFHPQEVSMVRNLIQKDRTFAVLAYSNVQ
EREAQFGTTAEIYAYREEQDFGIEIVKVKAIGRQRFKVLELRT
QSDGIQQAKVQILPECVLPSTMSAVQLESLNKCQIFPSKPVSR
EDQCSYKWWQKYQKRKFHCANLTSWPRWLYSLYDAETLMDRIK
KQLREWDENLKDDSLPSNPIDFSYRVAACLPIDDVLRIQLLKI
GSAIQRLRCELDIMNKCTSLCCKQCQETEITTKNEIFSLSLCG
PMAAYVNPHGYVHETLTVYKACNLNLIGRPSTEHSWFPGYAWT
VAQCKICASHIGWKFTATKKDMSPQKFWGLTRSALLPTIPDTE
DEISPDKVILCL

In some embodiments, CRBN used in the assays described herein is native human CRBN expressed from its genomic locus under its native promoter. In some embodiments, the native SALL4 is CRBN isoform 1. In some embodiments, the native CRBN is CRBN isoform 2. In some embodiments, the native CRBN is a mixture of CRBN isoforms 1 and 2.

In some embodiments, CRBN is recombinant human CRBN. Recombinant CRBN can be produced by the methods described above.

In some embodiments, CRBN, e.g., the native or recombinant human CRBN, 80, 81, 82, 83, 84, 85, 86, 87, 88, 89, 90, 91, 92, 93, 94, 95, 96, 97, 98, or 99% identity to SEQ ID NO: 2.

In some embodiments, CRBN, e.g., the native or recombinant human CRBN, is truncated at the N-terminus by 1-100 amino acids. In some embodiments, CRBN used in the assays described herein is truncated at the N-terminus by 1, 5, 10, 20, 30, 40, 50, 60, 70, 80, 90, or 100 amino acids. In some embodiments, a protein having the sequence of SEQ ID NO. 2 is truncated at the N-terminus by 1, 5, 10, 20, 30, 40, 50, 60, 70, 80, 90, or 100 amino acids.

In some embodiments, CRBN, e.g., the native or recombinant human CRBN, used in the assays described herein is truncated at the C-terminus by 1-100 amino acids. In some embodiments, CRBN used in the assays described herein is truncated at the C-terminus by 1, 5, 10, 20, 30, 40, 50, 60, 70, 80, 90, or 100 amino acids. In some embodiments, a protein having the sequence of SEQ ID NO. 2 is truncated at the C-terminus by 1, 5, 10, 20, 30, 40, 50, 60, 70, 80, 90, or 100 amino acids.

In some embodiments, the CRBN, e.g., the native or recombinant human CRBN, is truncated at the N-terminus and C-terminus by 1-100 amino acids. In some embodiments, CRBN used in the assays described herein is truncated at the N-terminus and C-terminus by 1, 5, 10, 20, 30, 40, 50, 60, 70, 80, 90, or 100 amino acids. In some embodiments, a protein having the sequence of SEQ ID NO. 2 is truncated at the N-terminus and C-terminus by 1, 5, 10, 20, 30, 40, 50, 60, 70, 80, 90, or 100 amino acids.

In some embodiments, the CRBN comprises a fragment of CRBN. In some embodiments, the CRBN is recombinant human CRBN and comprises a fragment of 10-100 consecutive amino acids of SEQ ID NO. 2, e.g., 10, 15, 20, 25, 30, 35, 40, 45, 50, 60, 70, 80, 90, or 100 amino acids of SEQ ID NO. 2.

In some embodiments, the CRBN is recombinant human CRBN and comprises 1-10 acid substitutions e.g., 2, 3, 4, 5, 6, 7, 8, 9, or 10 amino acid substitutions. In some embodiments, the CRBN has a mutation at V388 of SEQ ID NO. 2, e.g., a V388I mutation.

In some embodiments, the recombinant human CBRN used in the methods described herein is recombinantly expressed as a fusion with human DDB1 or a fragment thereof. As is used herein, “DDB1” is a polypeptide of 1140 amino acids encoding DNA damage-binding protein 1 having the sequence of NCBI Reference Sequence. NP_001914.3 (SEQ ID NO. 3).

In some embodiments, DDB1 is expressed N-terminal to CRBN. In some embodiments, DDB1 is expressed C-terminal to CRBN.

In some embodiments, DDB1, e.g., recombinant human DDB1, 80, 81, 82, 83, 84, 85, 86, 87, 88, 89, 90, 91, 92, 93, 94, 95, 96, 97, 98, or 99% identity to SEQ ID NO: 75.

In some embodiments, DDB1, e.g., the native or recombinant human DDB1, is truncated at the N-terminus by 1-100 amino acids. In some embodiments, DDB1 used in the assays described herein is truncated at the N-terminus by 1, 5, 10, 20, 30, 40, 50, 60, 70, 80, 90, or 100 amino acids. In some embodiments, a protein having the sequence of SEQ ID NO. 75 is truncated at the N-terminus by 1, 5, 10, 20, 30, 40, 50, 60, 70, 80, 90, or 100 amino acids.

In some embodiments, DDB1, e.g., the native or recombinant human DDB1, used in the assays described herein is truncated at the C-terminus by 1-100 amino acids. In some embodiments, DDB1 used in the assays described herein is truncated at the C-terminus by 1, 5, 10, 20, 30, 40, 50, 60, 70, 80, 90, or 100 amino acids. In some embodiments, a protein having the sequence of SEQ ID NO. 75 is truncated at the C-terminus by 1, 5, 10, 20, 30, 40, 50, 60, 70, 80, 90, or 100 amino acids.

In some embodiments, the DDB1, e.g., the native or recombinant human DDB1, is truncated at the N-terminus and C-terminus by 1-100 amino acids. In some embodiments, DDB1 used in the assays described herein is truncated at the N-terminus and C-terminus by 1, 5, 10, 20, 30, 40, 50, 60, 70, 80, 90, 100 amino acids. In some embodiments, a protein having the sequence of SEQ ID NO. 75 is truncated at the N-terminus and C-terminus by 1, 5, 10, 20, 30, 40, 50, 60, 70, 80, 90, or 100 amino acids.

In some embodiments, the DDB1 a fragment of DDB1. In some embodiments, the DDB1 is recombinant human DDB1 and comprises a fragment of 10-100 consecutive amino acids of SEQ ID NO. 75, e.g., 10, 15, 20, 25, 30, 35, 40, 45, 50, 60, 70, 80, 90, or 100 amino acids of SEQ ID NO. 75.

In some embodiments, the DDB1 is recombinant human DDB1 and comprises 1-10 amino acid substitutions e.g., 2, 3, 4, 5, 6, 7, 8, 9, or 10 amino acid substitutions. In some embodiments, the DDB1 is DDB1ΔB having a deletion or substitution of beta-propeller domain B. In some embodiments, comprises DDB1 of SEQ ID NO. 75 in which residues 396-705 are replaced, e.g., replaced with a linker having the sequence GNGNSG.

In some embodiments, CRBN or DDB1 used in the assays described herein is tagged with a detectable label. Examples of tags are well known in the art and include, e.g., HIS tags, biotin tags, streptavidin tags, Flag tags and GST tags. In some embodiments, CRBN used in the assays described herein is tagged with His. In some embodiments, CRBN used in the assays described herein is tagged with spycatcher or LgBiT.

Assays for Detecting Targeting of SALL4 to CRBN for Degradation

Described herein are a variety of assays for assessing the teratogenicity of an agent by detecting the targeting of SALL4 to CRBN for degradation. In some embodiments, SALL4 levels are measured. In some embodiments, the association between SALL4 and CRBN is measured. In some embodiments, SALL4 ubiquitination is measured. In some embodiments, SALL4 degradation products are measured.

In some embodiments, an agent is teratogenic if SALL4 levels are substantially decreased, if SALL4 is substantially associated with CRBN, if SALL4 is substantially ubiquitinated, or if SALL4 is substantially degraded relative to a control. As is used herein “substantially” means 20% more than a control, e.g., 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.5%, 99.9%, or 100% more than a control. In some embodiments, a compound is teratogenic if SALL4 levels are decreased 20% more than a control, e.g., 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.5%, 99.9%, or 100% more than a control. In some embodiments, a compound is teratogenic if SALL4 is ubiquitinated 20% more than a control, e.g., 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.5%, 99.9%, or 100% more than a control. In some embodiments, a compound is teratogenic if SALL4 is associated with CRBN 20% more than a control, e.g., 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.5%, 99.9%, or 100% more than a control. In some embodiments, a compound is teratogenic if SALL4 is degraded 20% more than a control, e.g., 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.5%, 99.9%, or 100% more than a control.

As is used herein, a control comprises measuring SALL4 levels, SALL4 ubiquitination, SALL4 association with CRBN, or SALL4 degradation, in the absence of the agent. In some embodiments, the control comprises identical, or near identical conditions as the conditions for measuring SALL4 levels, SALL4 ubiquitination, SALL4 association with CRBN, or SALL4 degradation in the presence of the agent. In some embodiments, identical, or near identical conditions comprises the same cell type. In some embodiments, identical, or near identical conditions comprises using cells from the same culture for expressing SALL4. In some embodiments, identical, or near identical conditions comprises using SALL4 obtained from the same protein isolation prep. In some embodiments, identical, or near identical conditions comprises using the same buffers, antibodies, or other reagents.

In some embodiments, cells expressing SALL4 for the assays described herein are murine cells. In some embodiments, cells expressing SALL4 for the assays described herein are rat cells. In some embodiments, cells expressing SALL4 for the assays described herein are rabbit cells. In some embodiments, cells expressing SALL4 for the assays described herein are monkey cells. In some embodiments, cells expressing SALL4 for the assays described herein are zebrafish cells. In some embodiments, cells expressing SALL4 for the assays described herein are human cells.

Cells can be cultured according to art known cell culture methods. For example, cells can be cultured in DMEM, RPMI1640, KO-DMEM, Essential 8, or StemFlex media. In some embodiments, cells are cultured in media supplemented with FBS. In some embodiments, cells are cultured in media supplemented with glutamine. In some embodiments, cells are cultured in media supplemented with non-essential amino acids. In some embodiments, cells are cultured in media supplemented with HEPES, sodium pyruvate, 2-mercaptoethanol, antibiotics, and/or mLIF.

In some embodiments, the cell expressing SALL4 is contacted with the agent. In some embodiments, SALL4 is contacted with the agent after isolation from the cell expressing SALL4.

In some embodiments, a cell expressing SALL4 is contacted with the agent for 2, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 38, 40, 42, 44, 46, or 48 or more hours.

In some embodiments, a cell expressing SALL4 is contacted with the agent at a concentration of 0.01 μM to 1,000 μM. In some embodiments, a cell expressing SALL4 is contacted with the agent at a concentration of 0.01 μM, 0.05 μM, 0.1 μM, 0.2 μM, 0.3 μM, 0.4 μM, 0.5 μM, 0.6 μM, 0.7 μM. 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 200 μM, 300 μM, 400 μM, 500 μM, 600 μM, 700 μM, 800 μM, 900 μM, or 1000 μM. In some embodiments, a cell expressing SALL4 is contacted with the agent at a concentration of 0.05 μM to 100 μM. In some embodiments, a cell expressing SALL4 is contacted with the agent at a concentration of 0.1 μM to 20 μM.

In some embodiments, SALL4 levels, SALL4 ubiquitination, SALL4 association with CRBN, or SALL4 degradation are measured using assays used for protein detection. Assays for detecting protein levels include, but are not limited to, immunoassays (also referred to herein as immune-based or immuno-based assays, e.g., Western blot, ELISA, proximity extension assays, and ELISpot assays), Mass spectrometry, and multiplex bead-based assays. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

In some embodiments, SALL4 degradation is measured by visualizing SALL4 levels in a living cell.

In some embodiments, SALL4 degradation is measured by detecting SALL4 association with CRBN by FRET. As used herein the term “Förster Resonance Energy Transfer” or “FRET” refers to an energy transfer mechanism occurring between two fluorescent molecules: a fluorescent donor and a fluorescent acceptor (i.e., a FRET pair) positioned within a range of about 1 to about 10 nanometers of each other wherein one member of the FRET pair (the fluorescent donor) is excited at its specific fluorescence excitation wavelength and transfers the fluorescent energy to a second molecule, (fluorescent acceptor) and the donor returns to the electronic ground state. In some embodiments, the FRET is TR-FRET (time-resolved fluorescence energy transfer). TR-FRET is the practical combination of time-resolved fluorometry (TRF) with FRET. TR-FRET combines the low background aspect of TRF with the homogeneous assay format of FRET.

SALL4 Levels

In some embodiments, SALL4 levels are measured in cells in the presence of an agent, and substantially reduced levels of SALL4 in the presence of the agent, relative to in the absence of the agent, is indicative of SALL4 degradation, e.g., teratogenicity of the agent.

In some embodiments, SALL4, e.g, a cell expressing SALL4, is contacted with an agent and the level of SALL4 is measured. In some embodiments, the level of SALL4 in cells contacted with the agent is compared to the level of SALL4 in cells that are not contacted with the agent.

In some embodiments, the level of SALL4 is measured in extracts from the cell. In some embodiments, cell extracts are prepared by lysing the cells, e.g., mechanically or chemically. In some embodiments, the cell lysate is homogenized, e.g., by passing through a needle. In some embodiments, the homogenized cell lysate is clarified, e.g., by centrifugation.

In some embodiments, the level of SALL4 is measured by running protein from the cell on an SDS-PAGE gel, transferring the protein to a solid support, and probing the solid support with an anti-SALL4 antibody, e.g., by western blotting.

In some embodiments, SALL4 is tagged with a detectable label and the level of SALL4 is measured by running protein from the cell on an SDS-PAGE gel, transferring the protein to a solid support, and probing the solid support with an antibody to the detectable label.

In some embodiments, SALL4 levels are measured by Western Blot. Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that preferentially bind to SALL4 (e.g., anti-SALL4) can then be used to assess SALL4 levels and/or to visualize SALL4 degradation products, where the intensity of the signal from the detectable label corresponds to the amount of SALL4 present. Levels can be quantitated, for example by densitometry.

In some embodiments, SALL4 levels are measured by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). see, e.g., U.S. Publication Nos. 20030199001, 20030134304, and 20030077616.

Mass spectrometry methods are well known in the art and have been used to quantify and/or identify proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In some embodiments, SALL4 levels are measured by fusing SALL4 to a detectable label and visualizing the level of SALL4 in cells. In some embodiments, the level of SALL4 fused to a detectable label visualized in cells contacted with an agent is compared to level of SALL4 fused to a detectable agent visualized in cells that are not contacted to the agent.

In some embodiments, a cell expressing SALL4 fused to a detectable label is expressed in cells also expressing a second detectable label. In some embodiments, the level of SALL4 fused to a detectable label is standardized relative to the level of the second detectable label.

In some embodiments, the level of SALL4 fused to a detectable label is visualized in live cells. In some embodiments, the level of SALL4 fused to a detectable label is visualized in cells that have been fixed after the cells have been contacted with the agent. Methods for fixing cells are well known in the art.

Examples of detectable labels are known in the art and include, for example, a His-tag, a myc-tag, an S-peptide tag, a MBP tag, a GST tag, a FLAG tag, a thioredoxin tag, a GFP tag, a CFP tag, an RFP tag, a YFP tag, a BCCP, a calmodulin tag, a Strep tag, an HSV-epitope tag, a V5-epitope tag, a CBP tag or components of the nanoBiT system, e.g., HiBiT, LoBiT, LgBiT, SmBiT.

In some embodiments, the levels of SALL4 fused to a detectable label is visualized by microscopy. Microscopic methods are well known in the art and include, e.g., phase contrast microscopy, fluorescence microscopy, and confocal microscopy.

In some embodiments, the levels of SALL4 fused to a detectable label is determined by FACS.

SALL4 Degradation Products

In some embodiments, SALL4 degradation products are measured in cells in the presence of an agent, and substantial degradation of SALL4 in the presence of the agent, relative to in the absence of the agent, is indicative of teratogenicity of the agent.

In some embodiments, SALL4 degradation products are detected by Western Blot, as is described supra.

In some embodiments, SALL4, e.g, a cell expressing SALL4, is contacted with an agent and the degradation products of SALL4 are measured. In some embodiments, the degradation products of SALL4 in cells contacted with the agent is compared to the degradation products of SALL4 in cells that are not contacted with the agent. In some embodiments, the degradation products of SALL4 are measured by running protein from the cell on an SDS-PAGE gel, transferring the protein to a solid support, and probing the solid support with an anti-SALL4 antibody.

In some embodiments, SALL4 is tagged with a detectable label and the degradation products of SALL4 are measured by running protein from the cell on an SDS-PAGE gel, transferring the protein to a solid support, and probing the solid support with an antibody to the detectable label.

In some embodiments, SALL4 degradation products are detected by mass spectrometry, as is described herein.

SALL4-CRBN Association

In some embodiments, SALL4 association with CRBN is measured in cells in the presence of an agent, and substantial association of SALL4 with CRBN in the presence of the agent, relative to in the absence of the agent, is indicative of SALL4 degradation, e.g., teratogenicity of the agent.

In some embodiments, SALL4 association with CRBN is measured by co-immunoprecipitation assay. Methods for immunoprecipitation, e.g., co-immunoprecipitation are well known in the art and comprise contacting a first antibody attached to a solid support with cell lysate to immunoprecipitate a first protein recognized by the antibody. In some embodiments, the immunoprecipitated protein is run on an SDS-PAGE gel, transferred to a solid support, and probed with a second antibody to a second protein, e.g., a western is performed, to determine if the second protein binds, e.g., is immunoprecipitated with, the first protein. In some embodiments, mass spectrometry, as described supra, is performed on the immunoprecipitation reaction to detect SALL4 association with CRBN.

In some embodiments, the first protein is SALL4 and the second protein is CRBN. In other embodiments, the first protein is CRBN and the second protein is SALL4.

In some embodiments, the first antibody is an anti-SALL4 antibody. In some embodiments, the second antibody is an anti-CRBN antibody. In some embodiments, the first antibody is an anti-CRBN antibody. In some embodiments, the second antibody is an anti-SALL4 antibody.

In some embodiments, SALL4 and/or CRBN are tagged with a detectable label. In some embodiments, SALL4 or CRBN is tagged with a detectable label and is immunoprecipitated using an antibody against the label. In some embodiments, SALL4 or CRBN is tagged with a detectable label and the solid support is probed with the antibody against the label.

In some embodiments, the interaction between SALL4 and CRBN is tested when SALL4 and/or CRBN are contacted with the agent ex vivo, e.g., after isolation of SALL4 and/or CRBN from cells. In some embodiments, the interaction between SALL4 and CRBN is tested by ELISA. In some embodiments, the interaction between SALL4 and CRBN is tested by FRET. In some embodiments, the FRET is TR-FRET.

In some embodiments, SALL4 and CRBN are incubated with the agent for 1, 2, 3, 4, 5, 6, 7, 8, 9, 10, 11, 12, 13, 14, 15, 16, 17, 18, 19, 20, 25, 30, 35, 40, 45, 50, 55, or 60 minutes or more.

In some embodiments, the agent is added at a concentration of log −10M, log −9M, log −8M, log −7M, log −6M, log −5M, log −4M, log −3M, log −2M, or log −1M.

In some embodiments, SALL4 is provided at a concentration of 1 nM-1 μM. In some embodiments, SALL4 is provided at a concentration of 1 nM, 10 nM, 20 nM, 30 nM, 40 nM, 50 nM, 60 nM, 70 nM, 80 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, or 1000 nM. In some embodiments, SALL4 is provided at a concentration of 10 nM-300 nM. In some embodiments, SALL4 is provided at a concentration of 50 nM-200 nM.

In some embodiments, CRBN is provided at a concentration of 500 nm-500 μM. In some embodiments, CRBN is provided at a concentration of 500 nm, 600 nm, 700 nm, 800 nm, 900 nm, 910 nm, 920 nm, 930 nm, 940 nm, 950 nm, 960 nm, 970 nm, 980 nm, 990 nm, 991 nm, 992 nm, 993 nm, 994 nm, 995 nm, 996 nm, 997 nm, 998 nm, 999 nm, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, or 500 μM. In some embodiments, CRBN is provided at a concentration of 900 nm-100 μM.

In some embodiments, the interaction between SALL4 and CRBN is tested by ELISA. For example, a first molecule, e.g., SALL4 or CRBN, is contacted to a microtiter plate whose bottom surface has been coated with a second molecule, e.g., a limiting amount of a second molecule, e.g., SALL4 or CRBN, in the presence and in the absence of the agent. The plate is washed with buffer to remove non-specifically bound polypeptides. Then the amount of the binding protein bound to the target on the plate is determined by probing the plate with an antibody that can recognize the binding protein. The antibody is linked to a detection system (e.g., an enzyme such as alkaline phosphatase or horse radish peroxidase (HRP) which produces a colorimetric product when appropriate substrates are provided).

In some embodiments, the interaction between SALL4 and CRBN is tested by FRET (fluorescence energy transfer) (see, for example, Lakowicz et al., U.S. Pat. No. 5,631,169; Stavrianopoulos, et al., U.S. Pat. No. 4,868,103). A fluorophore label on the first molecule, e.g., SALL4 or CRBN, is selected such that its emitted fluorescent energy can be absorbed by a fluorescent label on a second molecule (e.g., SALL4 or CRBN) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means, e.g., using a fluorimeter. By titrating the amount of the first or second binding molecule, a binding curve can be generated to estimate the equilibrium binding constant.

In some embodiments, the FRET is TR-FRET (time-resolved fluorescence energy transfer). TR-FRET is the practical combination of time-resolved fluorometry (TRF) with FRET. TR-FRET combines the low background aspect of TRF with the homogeneous assay format of FRET.

Donor acceptor pairings for TR-FRET are well known in the art and include, e.g., Europium (donor) and Allophycocyanin (acceptor), Terbium (donor) and Phycoerythrin (acceptor), and Terbium (donor) and BODIPY (acceptor).

SALL4 Ubiquitination

In some embodiments, SALL4 ubiquitination is measured in cells in the presence of an agent, and substantial ubiquitination of SALL4 in the presence of the agent, relative to in the absence of the agent, is indicative of SALL4 degradation, e.g., teratogenicity of the agent.

In some embodiments, SALL4 ubiquitination is measured by Western Blot, as is described supra.

In some embodiments, SALL4, e.g, a cell expressing SALL4, is contacted with an agent and the ubiquitination of SALL4 is measured. In some embodiments, the ubiquitination of SALL4 in cells contacted with the agent is compared to the ubiquitination of SALL4 in cells that are not contacted with the agent. In some embodiments, the ubiquitination of SALL4 is measured by running protein from the cell on an SDS-PAGE gel, transferring the protein to a solid support, and probing the solid support with an anti-ubiquitin antibody.

In some embodiments, ubiquitinated SALL4 is detected by mass spectrometry, as is described herein.

Antibodies

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

In some embodiments, the anti-SALL4 antibody used in the methods described herein specifically binds to SALL4 or an epitope thereof. In some embodiments, the anti-SALL4 antibody is reactive to human SALL4. In some embodiments, the anti-SALL4 antibody used in the methods described herein is ab57577 (Abcam). In some embodiments, the anti-SALL4 antibody used in the methods described herein is reactive to murine SALL4. In some embodiments, the anti-SALL4 antibody used in the methods described herein is ab29112 (Abcam). In some embodiments, the anti-SALL4 antibody used in the methods described herein is sc-101147 (Santa Cruz Biotechnology). In some embodiments, the anti-SALL4 antibody used in the methods described herein is 720030 (Thermo Fisher). In some embodiments, the anti-SALL4 antibody used in the methods described herein is PA5-29072 (Thermo Fisher). In some embodiments, the anti-SALL4 antibody used in the methods described herein is PA5-11566 (Thermo Fisher). In some embodiments, the anti-SALL4 antibody used in the methods described herein is 5850 (Cell Signaling Technology). In some embodiments, the anti-SALL4 antibody used in the methods described herein is MAB6374 (MD Systems).

In some embodiments, the anti-CRBN antibody used in the methods described herein specifically binds to CRBN or an epitope thereof. In some embodiments, the anti-CRBN antibody used in the methods described herein is BP1-91810 (Novus Biologicals). In some embodiments, the anti-CRBN antibody used in the methods described herein is ab68763 (abcam). In some embodiments, the anti-CRBN antibody used in the methods described herein is PA5-38037 (Thermo Fisher). In some embodiments, the anti-CRBN antibody used in the methods described herein is SAB1407456 (Sigma Aldrich). In some embodiments, the anti-CRBN antibody used in the methods described herein is HPA045910 (Sigma Aldrich). In some embodiments, the anti-CRBN antibody used in the methods described herein is HPA045910 11435-1-AP (Proteintech).

Anti-ubiquitin antibodies are well known in the art. Examples of anti-ubiquitin antibodies include, e.g., U5379 (Sigma-Aldrich), U0508 (Sigma-Aldrich), ab7780 (abcam), 3933 (Cell Signaling Technology), and 3936 (Cell Signaling Technology).

In some embodiments, the antibody used in the methods described herein specifically binds a detectable label described herein. Antibodies to detectable labels are extensively characterized in the art (see, e.g., Epitope Tags in Protein Research, Tag Selection & Immunotechniques, Sigma Life Sciences, 2012).

Agents

In some embodiments, the agent is an Immunomodulatory Imide Drug (IMiD). The term “immunomodulatory drug” or “IMiD” refers to a class of drugs that modifies the immune system response or the functioning of the immune system, such as by the stimulation of antibody formation and/or the inhibition of peripheral blood cell activity, and include, but are not limited to, thalidomide (α-N-phthalimido-glutarimide) and its analogues, REVLIMID® (lenalidomide), ACTI-MID™ (pomalidomide), OTEZLA® (apremilast), and pharmaceutically acceptable salts or acids thereof. The term “thalidomide” refers to drugs or pharmaceutical formulations comprising the active thalidomide compound 2-(2,6-dioxopiperidin-3-yl)-1H-isoindole-1,3(2H)-dione. Thalidomide derivatives thereof refer to structural variants of thalidomide that have a similar biological activity such as, for example, without limitation, lenalidomide (REVLEVHD™) ACTEVIID™ (Celgene Corporation), and POMALYST™ (Celgene Corporation), and the compounds disclosed in U.S. Pat. No. 5,712,291, WO02068414, and WO2008154252, each of which is incorporated herein by reference in its entirety. Illustrative examples of EVIiDs that may be administered with the compositions contemplated herein include, but are not limited to, thalidomide, lenalidomide, pomalidomide, linomide, CC-1088, CDC-501, and CDC-801.

As is shown in Example 1, thalidomide, lenalidomide, and pomalidomide all induce degradation of SALL4. IMiDs that do not induce degradation of SALL4 are also identified, as is shown in Example 2, including DFCI1-DFCI2.

In some embodiments, the agent is a PROTAC (proteolysis targeting chimeras)/degrader. As is used herein, “PROTAC” or “degrader” refers to a bifunctional compound that comprises a moiety for binding a target protein to be degraded (e.g., a moiety that binds SALL4) linked to an E3 ubiquitin ligase binding moiety. In some embodiments, the E3 ubiquitin ligase binding moiety is a small molecule, e.g. IMiDs (e.g., thalidomide, lenalidomide). In some embodiments, the moiety for binding the target protein is a small molecule. In some embodiments, the E3 ubiquitin ligase binding moiety is attached to the moiety for binding the target protein via a linker. In some embodiments, the linker is a bond or a chemical linking moiety. PROTACs/degraders are described e.g., in U.S. patent application U.S. Ser. No. 14/792,414, filed Jul. 6, 2015; U.S. Ser. No. 14/707,930, filed May 8, 2015; US20180147202; US20180125821 US20160045607; and US20180050021, U.S. Pat. Nos. 9,821,068, 9,750,816, 9,770,512, and 9,694,084, each of which is incorporated herein by reference). PROTACs/degraders, are a new therapeutic strategy recently developed to reduce and/or eliminate proteins associated with certain pathological states by creating bifunctional compounds that recruit E3 ubiquitin ligase to a target protein, which subsequently induce ubiquitination and proteasome-mediated degradation of the target protein. E3 ubiquitin ligases are proteins that, in combination with an E2 ubiquitin-conjugating enzyme, promote the attachment of ubiquitin to a lysine of a target protein via an isopeptide bond (e.g., an amide bond that is not present on the main chain of a protein). In some embodiments, the E3 ubiquitin ligase is CRBN. The ubiquitination of the protein results in degradation of the target protein by the proteasome. PROTACs/degraders employ a strategy of recruiting a target protein to an E3 ubiquitin ligase and subsequently inducing proteasome-mediated degradation of the target protein. The bifunctional compounds can induce the inactivation of a protein of interest upon addition to cells or administration to an animal, and could be useful as biochemical reagents, leading to a new paradigm for disease treatment by removing pathogenic or oncogenic proteins (See Crews C., et al., Chemistry & Biology, 2010, 17(6):551-555; Schnnekloth J S Jr., Chembiochem, 2005, 6(1):40-46). An exemplary PROTAC/degrader involves a bifunctional compound which links a binder of BRD4 (a protein from the bromodomain and extraterminal domain (BET) family) with an E3 ligase cereblon (CRBN) binding moiety (pomalidomide). See Lu J., Qian Y., Altieri M., Crews, C., et al., Chemistry & Biology. 2015; 22(6):755-763. Another exemplary PROTAC/degrader is a degronomid, which involves a bifunctional compound that links a binder of a protein from the bromodomain and extraterminal domain (BET) family (e.g., BRD2, BRD3, or BRD4) with an E3 ligase cereblon binding moiety (e.g., phthalimide). See Winter, G. E., Bradner, J. E., et al., Science (New York, NY). 2015; 348(6241):1376-1381.

In some embodiments, the agent is a pesticide. Pesticides are well known in the art. Exemplary pesticides include, e.g., acaricides, algicides, antifeedants, avicides, bactericides, bird repellents, chemosterilants, herbicide safeners, insect attractants, insect repellents, insecticides, mammal repellents, mating disruptors, molluscicides, nematicides, plant activators, plant-growth regulators, rodenticides, synergists, and virucides. Exemplary microbial pesticides include Bacillus thuringiensis and mycorrhizal fungi. Exemplary insecticides include, but are not limited to, thiodan, diazinon, and malathion. Exemplary commercially available pesticides include, but are not limited to. Admire™ (imidacloprid) manufactured by Bayer, Regent™ (fipronil) manufactured by BASF, Dursban™ (chlorpyrifos) manufactured by Dow, Cruiseru (thiamethoxam) manufactured by Syngenta, Karate™ (lambda-cyhalothrin) manufactured by Syngenta, and Decis™ (deltamethrin) manufactured by Bayer.

### EXAMPLES

### Example 1: CRL4CN Dependent Degradation of SALL4 Underlies Thalidomide Teratogenicity

Frequently used to treat morning sickness, the drug thalidomide led to the birth of thousands of children with severe birth defects. Despite their teratogenicity, thalidomide and related IMiD drugs are now a mainstay of cancer treatment, however, the molecular basis underlying the pleiotropic biology and characteristic birth defects remains unknown. Here it is shown that IMiDs disrupt a broad transcriptional network through induced degradation of several C2H2 zinc finger transcription factors, including SALL4, a member of the spalt-like family of developmental transcription factors. Strikingly, heterozygous loss of function mutations in SALL4 result in a human developmental condition that phenocopies thalidomide induced birth defects such as absence of thumbs, phocomelia, defects in ear and eye development, and congenital heart disease. It is found that thalidomide induces degradation of SALL4 exclusively in humans, primates and rabbits, but not in rodents or fish, providing a mechanistic link for the species-specific pathogenesis of thalidomide syndrome.

Thalidomide was first marketed in the 1950s as a nonaddictive, nonbarbiturate sedative with anti-emetic properties, and widely used to treat morning sickness in pregnant women. Soon after its inception, reports of severe birth defects appeared, but were denied to be linked to thalidomide. Only in 1961, two independent reports confirmed that thalidomide was causative to this largest preventable medical disaster in modern history (Lenz, 1962; McBride, 1961). In addition to thousands of children born with severe birth defects, there were reports of increased miscarriage rates during this period (Lenz, 1988). Despite this tragedy, thalidomide, and its close derivatives, lenalidomide and pomalidomide, known as immunomodulatory drugs (IMiDs), are commonly used to treat a variety of clinical conditions such as multiple myeloma (MM) and 5q-deletion associated myelodysplastic syndrome (del(5q)-MDS) (D'Amato et al., 1994; Pan and Lentzsch, 2012).

While a potentially transformative treatment for MM, the molecular mechanisms of thalidomide teratogenicity, and many of its biological activities remain elusive. It was only recently shown that thalidomide and analogs exert their therapeutic effect by binding to the Cullin RING E3 ubiquitin ligase CUL4-RBX1-DDB1-CRBN (CRL4^{CRBN}) (Chamberlain et al., 2014; Fischer et al., 2014; Ito et al., 2010) and promote ubiquitination and degradation of key efficacy targets (neo-substrates), such as the zinc finger (ZnF) transcription factors IKAROS (IKZF1), AIOLOS (IKZF3), and ZFP91 (An et al., 2017; Fischer et al., 2014; Gandhi et al., 2014b; Kronke et al., 2014; Lu et al., 2014). IMiDs can also promote degradation of targets that lack a zinc finger domain, including Casein Kinase 1 alpha (CSNK1A1) (Kronke et al., 2015; Petzold et al., 2016) and GSPT1 (Matyskiela et al., 2016). CRL4^{CRBN} has further been implicated in the IMiD independent turnover of GLUL, BSG, and MEIS2 (Eichner et al., 2016; Kronke et al., 2014; Nguyen et al., 2016) and regulation of AMPK (Lee et al., 2013), processes potentially inhibited by IMiDs. While no obvious sequence homology exists between the known IMiD-dependent CRL4^{CRBN} substrates, all share the characteristic β-hairpin loop structure observed in X-ray crystal structures of IMiDs bound to CRBN and CSNK1A1 or GSPT1 (Matyskiela et al., 2016; Petzold et al., 2016), and a key glycine residue that engages the phthalimide moiety of IMiDs (An et al., 2017; Matyskiela et al., 2016; Petzold et al., 2016). Despite the progress in understanding the therapeutic mechanism of action of thalidomide, the cause of thalidomide syndrome has remained unknown since its description in 1961. Over the last 60 years, multiple theories such as anti-angiogenic properties or the formation of reactive oxygen species (ROS) by thalidomide, or specific metabolites of thalidomide have been linked to thalidomide induced defects. However, rarely they explain the full spectrum of birth defects caused by all members of the IMiD family of drugs (Vargesson, 2015). Moreover, it was shown that species such as mice, rats and bush babies are resistant to thalidomide induced teratogenicity (Butler, 1977; Heger et al., 1988; Ingalls et al., 1964; Vickers, 1967), which suggests an underlying genetic difference between species, more likely to be present in a specific substrate rather than in a general physiological mechanism such as anti-angiogenic effects or ROS production. To date, IMiD target identification efforts have largely focused on elucidating the mechanism of therapeutic efficacy of these drugs in MM and del(5q)-MDS (Gandhi et al., 2014a; Kronke et al., 2015; Kronke et al., 2014; Lu et al., 2014). These hematopoietic lineages may not express the specific proteins that are important in the developmental events disrupted by thalidomide during embryogenesis. In the absence of tractable animal models that closely resemble the human disease, human embryonic stem cells (hESC) were focused on as a model system that more likely expresses proteins relevant to embryo development, and set out to investigate the effects of thalidomide in this developmental context.

### Results

IMiDs Induce CRL4^{CRBN} Dependent Degradation of Multiple C₂H₂ Zinc Finger Transcription Factors

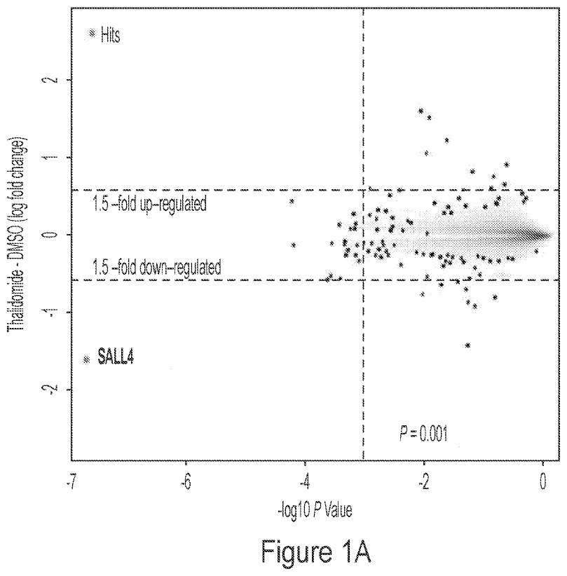

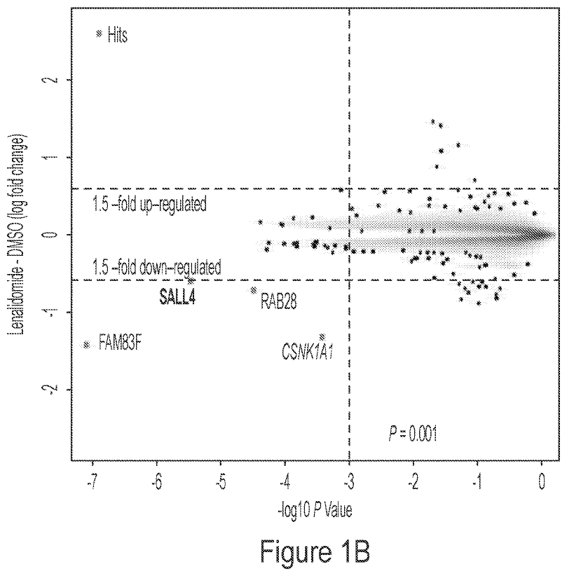

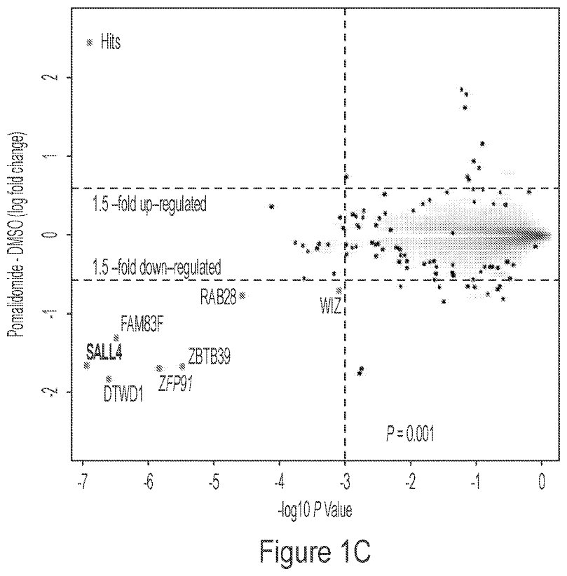

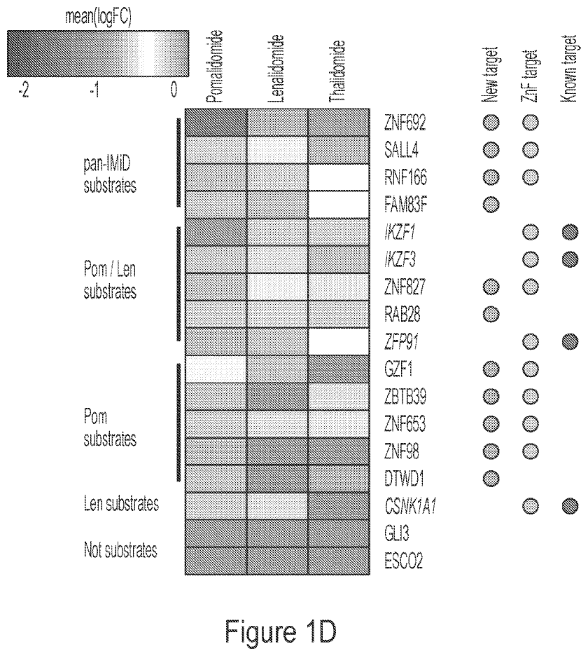

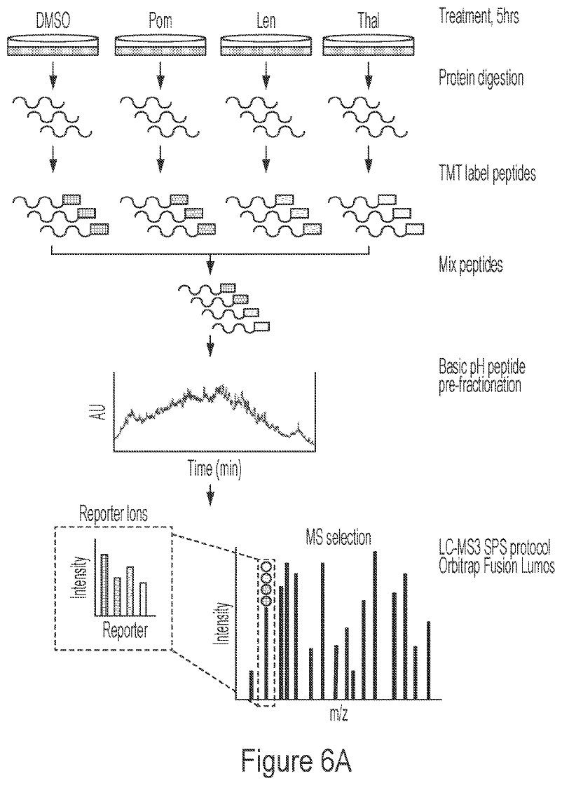

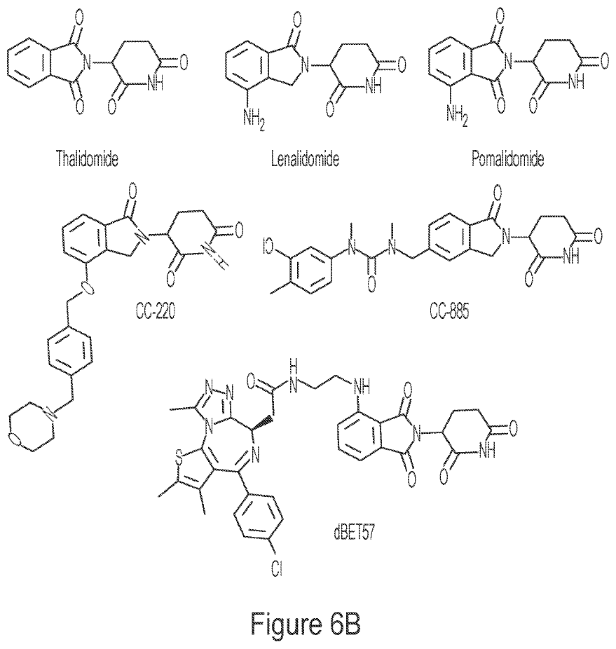

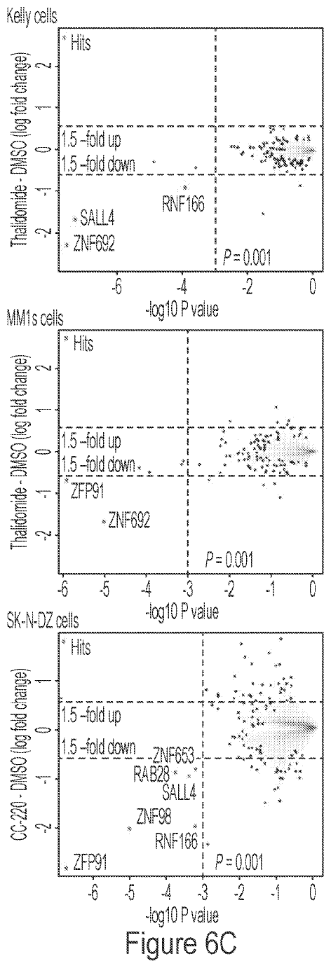

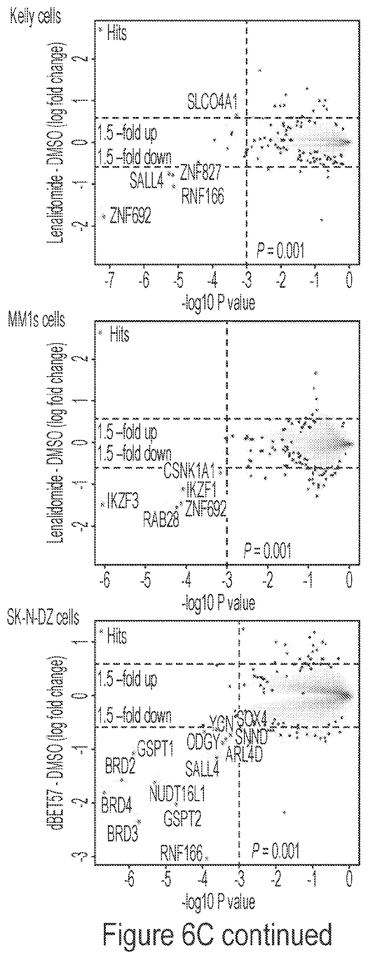

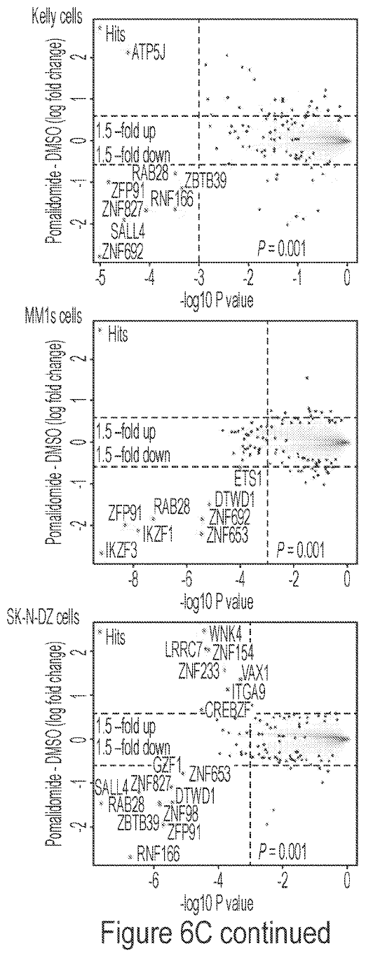

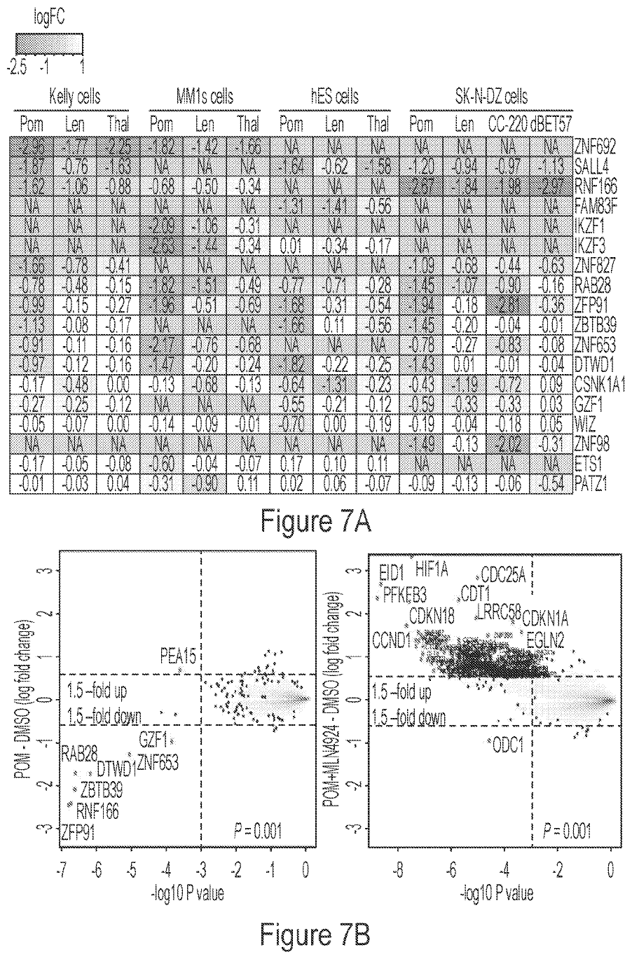

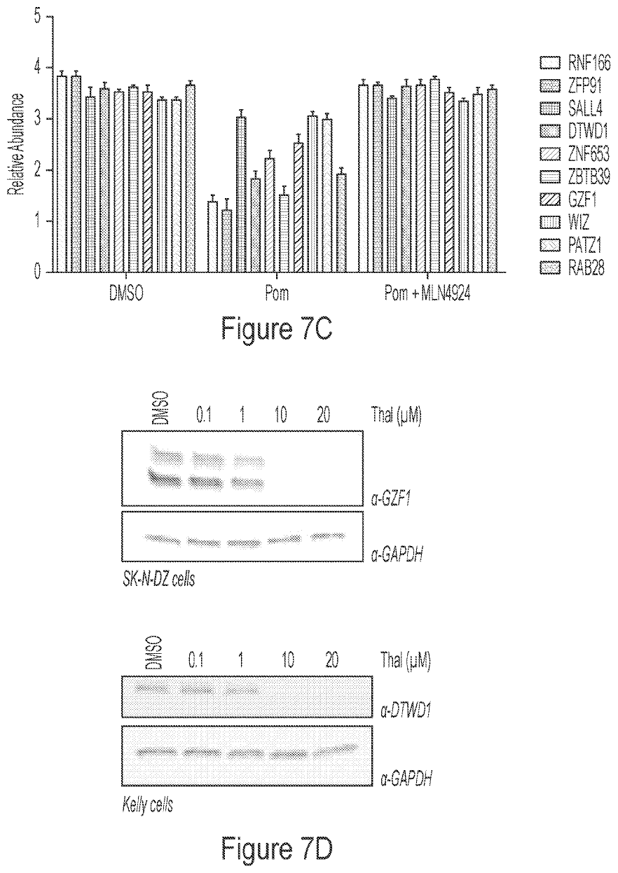

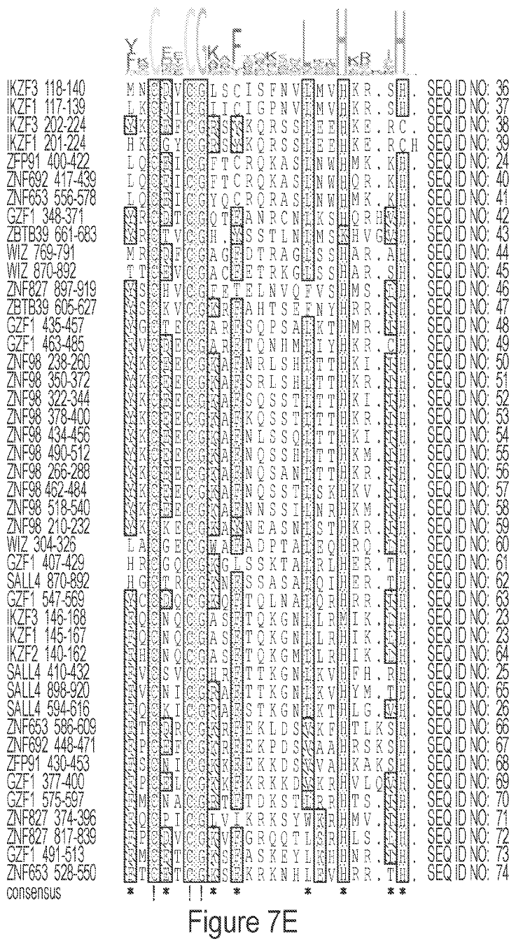

A mass spectrometry-based workflow was established (see FIGS. 6A-6C) to detect IMiD-induced protein degradation in hESC. To identify targets of IMiDs, cells were treated with 10 μM thalidomide, 5 μM lenalidomide, 1 μM of pomalidomide, or a DMSO control (see FIGS. 7A-7E). To minimize transcriptional changes and other secondary effects that often result from extended drug exposure (An et al., 2017), cells were treated for 5 hours and protein abundance was measured in multiplexed mass spectrometry-based proteomics using tandem mass tag (TMT) isobaric labels (McAlister et al., 2014) (see FIGS. 6A-6C and methods). From ˜10,000 proteins quantified in H9 hESC, only the developmental spalt-like transcription factor SALL4 showed statistically significant downregulation across all three drug treatments with a change in protein abundance greater than 1.5-fold, and a P Value <0.001 (FIGS. 1A-1C). In accordance with previous findings, it was also observed that treatment with lenalidomide led to degradation of CSNK1A1 (Kronke et al., 2015; Petzold et al., 2016). Pomalidomide induced degradation of additional targets including the previously characterized zinc finger protein ZFP91 (An et al., 2017), and the largely uncharacterized proteins ZBTB39, FAM83F, WIZ, RAB28, and DTWD1 (FIGS. 1A-1C).

This diverse set of neo-substrates observed in response to treatment with different IMiDs (number of substrates identified: Thal <Len<<Pom) prompted the further expansion of exploration of IMiD-dependent neo-substrates by profiling IMiDs in additional cell lines. Since degradation is mediated through CRL4^{CRBN}, and because CRBN expression levels are high in the central nervous system (CNS), the effects of IMiDs were assessed in two different neuroblastoma cell lines, Kelly and SK-N-DZ cells, as well as the commonly used multiple myeloma cell line, MM1s, as a control. Comprehensive proteomics studies across multiple independent replicates of hESC, Kelly, SK-N-DZ, and MM1s cells (FIGS. 1A-1D, see methods and FIGS. 6A-6C and FIGS. 7A-7E for details), revealed multiple novel substrates for IMiDs (ZNF692, SALL4, RNF166, FAM83F, ZNF827, RAB28, ZBTB39, ZNF653, DTWD1, ZNF98, and GZF1). In order to validate these novel targets, a ‘rescue’ proteomics experiment was carried out, in which SK-N-DZ cells were treated with 1 μM pomalidomide or with a co-treatment of 1 μM pomalidomide and 5 μM MLN4924 (a specific inhibitor of the NAE1/UBA3 Nedd8 activating enzyme). Inhibition of the Cullin RING ligase (CRL) by MLN4924 fully abrogated IMiD-induced degradation of targets (FIGS. 7B and 7C), and thereby confirmed the CRL dependent mechanism. This approach was confirmed by spot-checking IMiD-dependent degradation for novel targets for which antibodies were available by western blot (FIG. 7D). All targets that were found consistently degraded across multiple large sale proteomics experiments validated in those independent validation experiments, providing a high confidence target list (FIG. 1D).

Eight of the eleven new targets found in the proteomics screen are ZnF proteins (SALL4, ZNF827, ZBTB39, RNF166, ZNF653, ZNF692, ZNF98 and GZF1), and except for RNF166, all contain at least one ZnF domain that has the characteristic features previously described as critical for IMiD-dependent degradation (An et al., 2017) (FIG. 7E). A striking difference was also observed in substrate specificity between thalidomide, lenalidomide and pomalidomide (FIG. 1D). It is found that thalidomide induces robust degradation of the zinc finger transcription factors ZNF692, SALL4, and the ubiquitin ligase RNF166 in cell lines expressing detectable levels of those proteins (FIG. 1D and FIG. 7A). Lenalidomide results in additional degradation of ZNF827, FAM83F, and RAB28 along with the lenalidomide specific substrate CSNK1A1. Pomalidomide shows the most pronounced expansion of targets, and in addition induces robust degradation of ZBTB39, ZFP91, DTWD1, and ZNF653. It is interesting to note that DTWD1 is, as CSNK1A1 and GSPT1, another non zinc finger target that was found to be robustly degraded by pomalidomide. While this expansion of substrates is interesting and may contribute to some of the clinical differences between lenalidomide and pomalidomide, a target causative for teratogenicity would need to be consistently degraded across all IMiDs.

### SALL4, a Key Developmental Transcription Factor, is Bona Fide IMiD-Dependent CRL4^{CRBN} Target

The robust down-regulation of SALL4, a spalt-like developmental transcription factor important for limb development (Koshiba-Takeuchi et al., 2006), upon treatment with thalidomide, lenalidomide and pomalidomide prompted further investigation SALL4 as an IMiD-dependent target of CRL4^{CRBN}. Strikingly, human genetic research has shown that familial loss of function (LOF) mutations in SALL4 are causatively linked to the clinical syndromes, Duane Radial Ray syndrome (DRRS) also known as Okihiro syndrome, and mutated in some patients with Holt-Oram syndrome (HOS). Remarkably, both DRRS and HOS have large phenotypic overlaps with thalidomide embryopathy (Kohlhase et al., 2003), and this phenotypic resemblance has led to the misdiagnosis of patients with SALL4 mutations as cases of thalidomide embryopathy and the hypothesis that the tbx5/sall4 axis might be involved in thalidomide pathogenesis (Knobloch and Rtither, 2008; Kohlhase et al., 2003).

Thalidomide embryopathy is characterized not only by phocomelia, but also various other defects (Table 1), many of which are specifically recapitulated in syndromes known to originate from heterozygous LOF mutations in SALL4 (Kohlhase, 1993). The penetrance of DRRS in individuals with heterozygous SALL4 mutations likely exceeds 90% (Kohlhase, 2004), and thus partial degradation of SALL4 through IMiD exposure will likely result in similar clinical features observed in DRRS. All currently described SALL4 mutations are heterozygous LOF mutations, and the absence of homozygous mutations indicates the essentiality of the gene. Accordingly, homozygous deletion of Sall4 is early embryonic lethal in mice (Sakaki-Yumoto et al., 2006). Mice with heterozygous deletion of Sall4 show a high frequency of miscarriage, while surviving litters show ventricular septal defects and anal stenosis, both phenotypes that are observed in humans with DRRS or thalidomide syndrome (Sakaki-Yumoto et al., 2006). Mice carrying a heterozygous Sall4 genetrap allele show defects in heart and limb development, partially reminiscent to patients with DRRS or HOS (Koshiba-Takeuchi et al., 2006). Another genetic disorder with a related phenotype is Roberts Syndrome, caused by mutations in the ESCO2 gene (Afifi et al., 2016). While ESCO2 similarly encodes for a zinc finger protein and is transcriptionally regulated by ZNF143 (Nishihara et al., 2010), ESCO2 (as well as ZNF143, SALL1, SALL2, and SALL3) protein levels were found unchanged in all of the mass spectrometry experiments despite robust and ubiquitous expression (FIGS. 1D, 6A-6C, and 7A-7E).

TABLE 1
Common phenotypes in thalidomide syndrome, Duane
Radial Ray syndrome, and Holt-Oram syndrome.

| | Thalidomide syndrome | Duane Radial Ray syndrome | Holt-Oram syndrome |
|---|---|---|---|
| Upper limbs | | | |
| | Thumbs | Thumbs | Thumbs |
| | Radius | Radius | Radius |
| | Humerus | Humerus | Humerus |
| | Ulna | Ulna | Ulna |
| | Fingers | Fingers | Fingers |
| Lower limbs | | | |
| | Mostly normal lower limbs | Mostly normal lower limbs | |
| | Talipes dislocation | Talipes dislocation | |
| | Hip dislocation | | |
| | Shortening of long bones | | |
| Ears | | | |
| | Absence or abnormal pinnae | Abnormal pinnae | |
| | Deafness | Deafness | |
| | Microtia | | |
| Eyes | | | |
| | Colobomata | Colobomata | |
| | Microphthalmos | Microphthalmos | |
| | Abduction of the eye | Abduction of the eye | |
| | Duane anomaly | Duane anomaly | |
| Stature | | | |
| | Short stature | Postnatal growth retardation | |
| Heart | | | |
| | Ventricular septal defects | Ventricular septal defects | Ventricular septal defects |
| | Atrial septal defects | Atrial septal defects | Atrial septal defects |

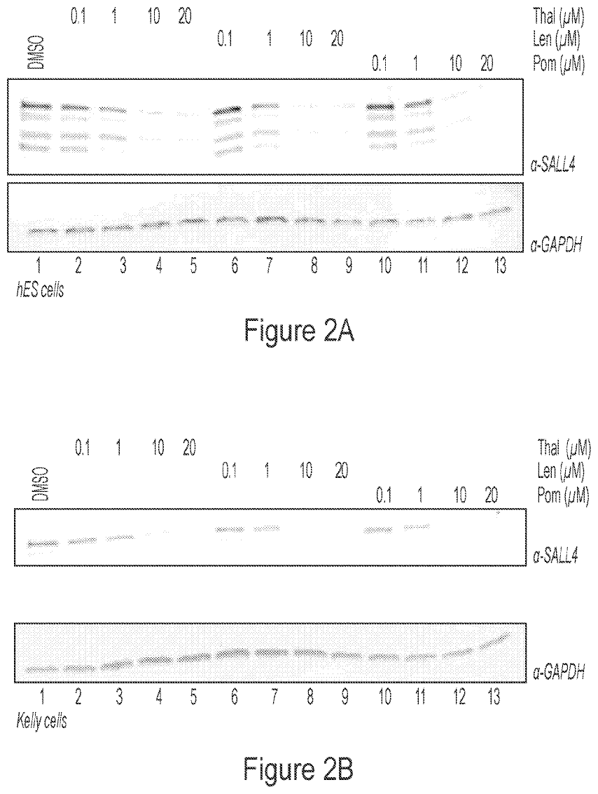

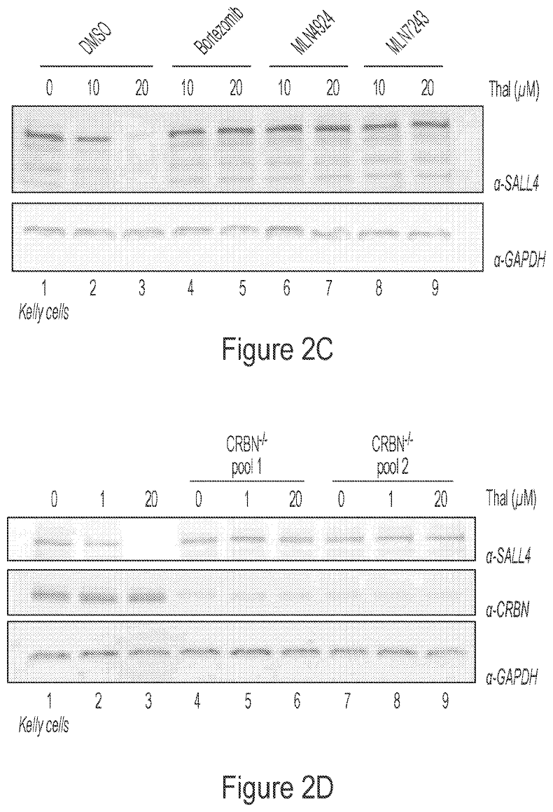

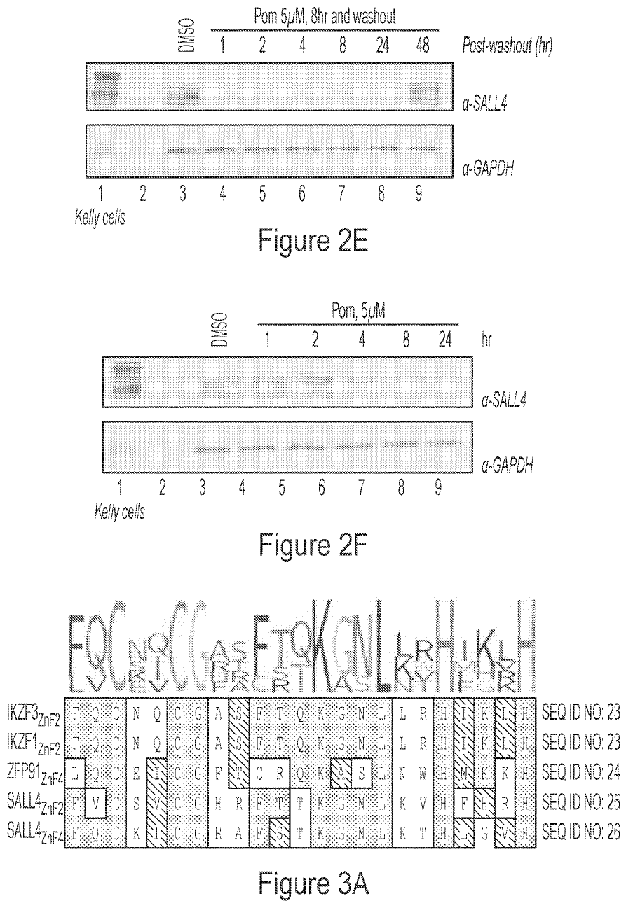

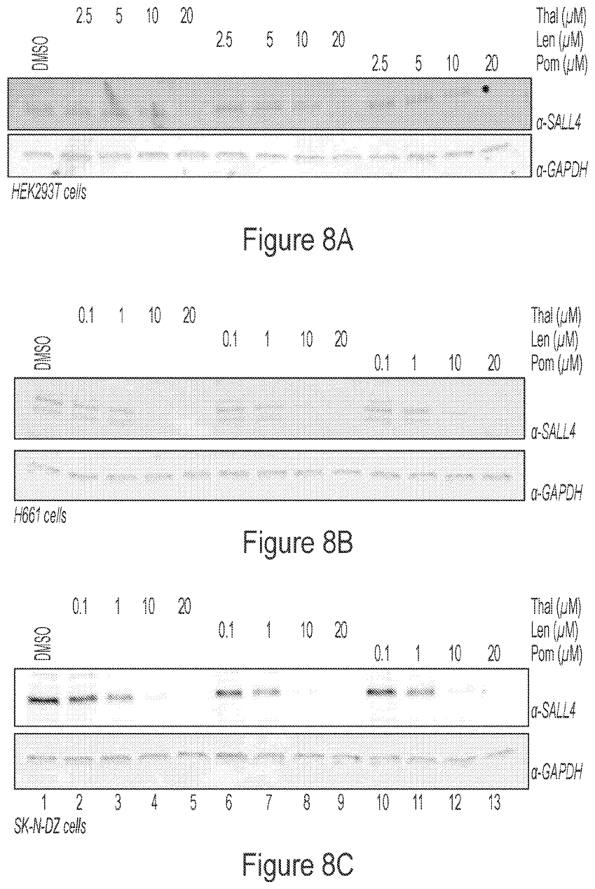

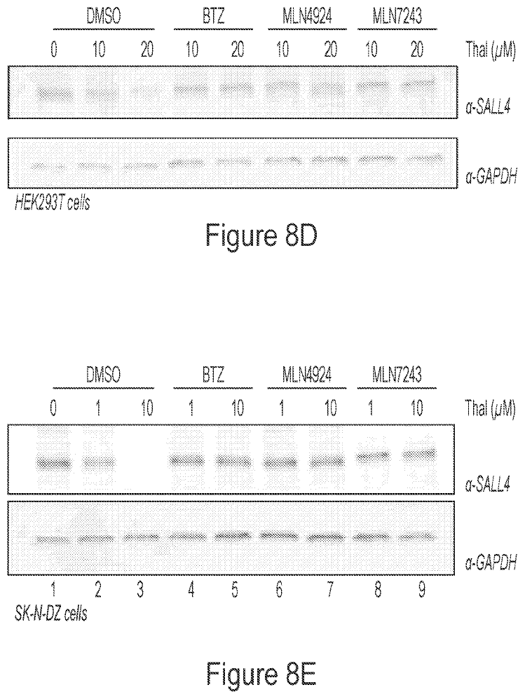

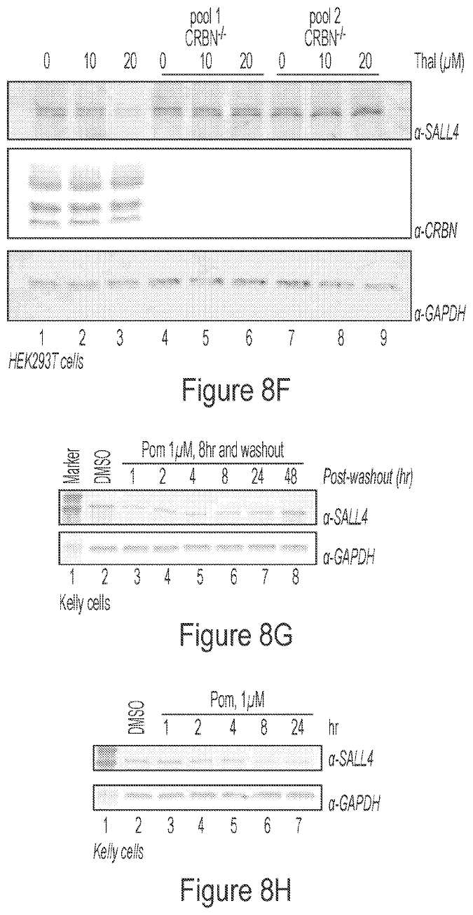

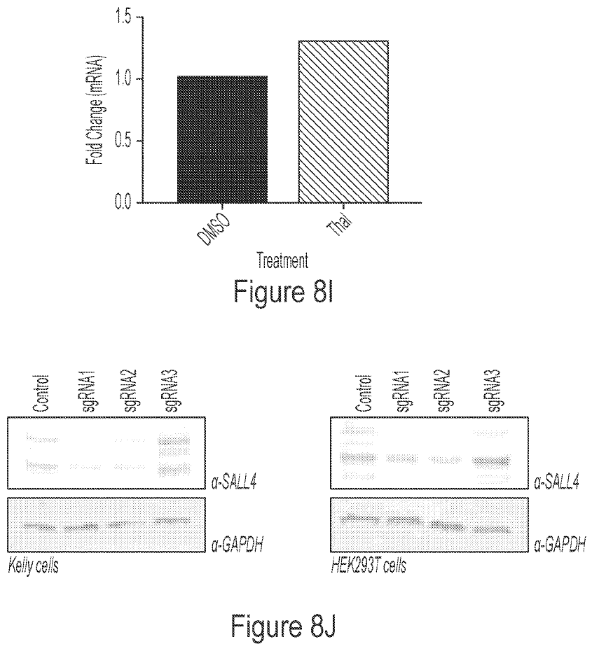

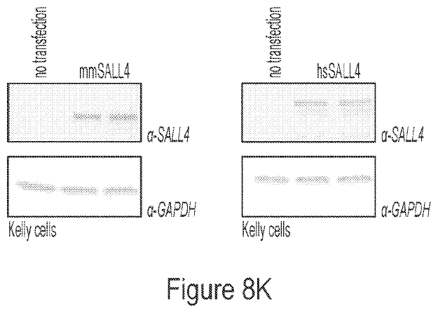

The remarkable phenotypic overlap of LOF mutations in SALL4 with thalidomide embryopathy led to further assessment of whether thalidomide and related IMiDs directly induce degradation of SALL4 in an IMiD and CRL4^{CRBN} dependent manner. To extend the mass spectrometry findings, H9 hESC was treated with increasing doses of thalidomide, lenalidomide, pomalidomide, or with DMSO as a control and assessed protein levels of SALL4 by western blot. A dose dependent decrease in protein levels was observed with all three drugs (FIG. 2A and FIGS. 8A-8K), in accordance with IMiD-induced protein degradation. qPCR was then used to confirm that thalidomide treatment does not reduce the level of SALL4 mRNA, but rather upregulates SALL4 mRNA, consistent with the protein-level changes being due to post-transcriptional effects (FIG. 8I).

Next, the robustness of SALL4 degradation across different lineages was assessed by subjecting a panel of cell lines (Kelly, SK-N-DZ, HEK293T, and H661 cells) to increasing concentrations of thalidomide, lenalidomide, pomalidomide, or DMSO as a control and performed western blot analysis (FIG. 2B and FIGS. 8A-8C). A dose-dependent decrease was observed in SALL4 protein levels with all three IMiD analogs and in all tested cell lines. In accordance with a CRL4^{CRBN} dependent mechanism, the IMiD-induced degradation was abrogated by co-treatment with the proteasome inhibitor bortezomib, the NEDD8 inhibitor MLN4924, or the ubiquitin E1 (UBA1) inhibitor MLN7243 (which blocks all cellular ubiquitination by inhibiting the initial step of the ubiquitin conjugation cascade) (FIG. 2C and FIGS. 8D-8E). To further evaluate the CRL4^{CRBN} dependent mechanism, CRBN^{-/-} Kelly and HEK293T cells were generated using CRISPR/Cas9 technology and treated the resulting CRBN-cells and parental cells with increasing concentrations of thalidomide, lenalidomide, or pomalidomide (FIG. 2D and FIG. 8F). In agreement with the CRBN dependent mechanism, no degradation of SALL4 was observed in CRBN^{-/-} cells. Thalidomide has a plasma half-life (t_{1/2}) of ˜6 to 8 hours (˜3 hours for lenalidomide, ˜9 hours for pomalidomide) and a maximum plasma concentration (Cₘₐₓ) of ˜5-10 μM (˜2.5 μM for lenalidomide, 0.05 μM for pomalidomide) upon a typical dose of 200-400 mg, 25 mg, or 2 mg for thalidomide, lenalidomide, or pomalidomide, respectively (Chen et al., 2017; Hoffmann et al., 2013; Teo et al., 2004). To recapitulate these effects in vitro, Kelly cells were treated with 1 or 5 μM pomalidomide for 8 hours, followed by washout of the drug and assessment of time dependent recovery of SALL4 protein levels (FIG. 2E and FIG. 8G). Treatment with pomalidomide induces degradation of SALL4 as early as 4 hours post-treatment (FIG. 2F and FIG. 8H), which recovered to levels close to pre-treatment level after 48 hours post-washout (FIG. 2E), together suggesting that a single dose of IMiD drugs will be sufficient to deplete SALL4 protein levels for >24 hours.

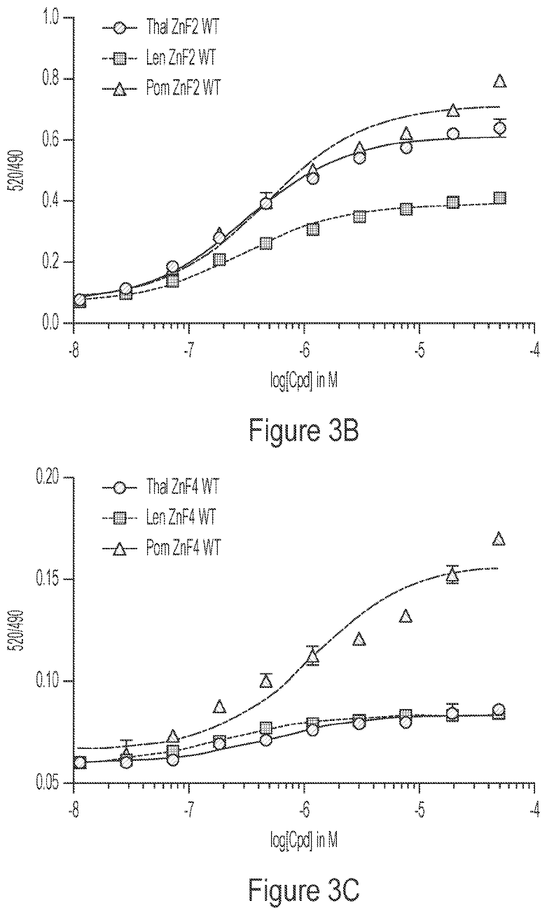

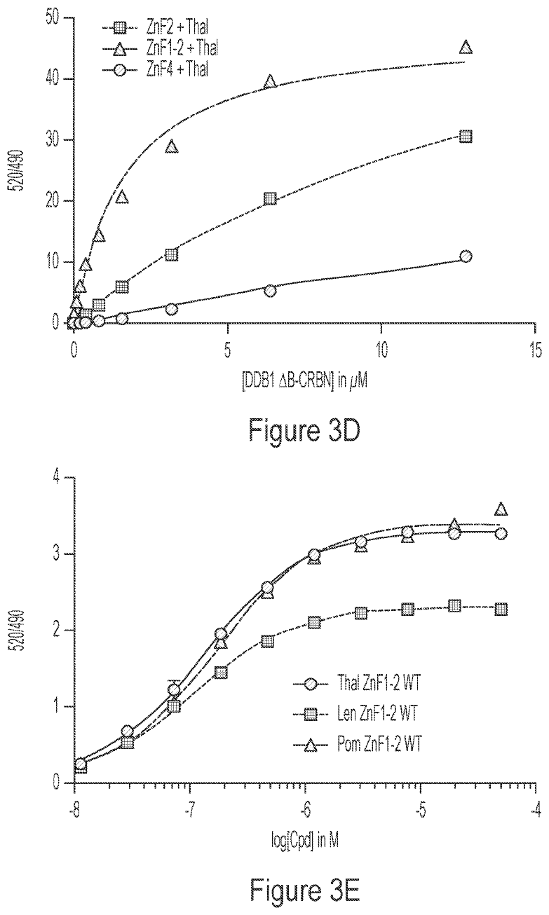

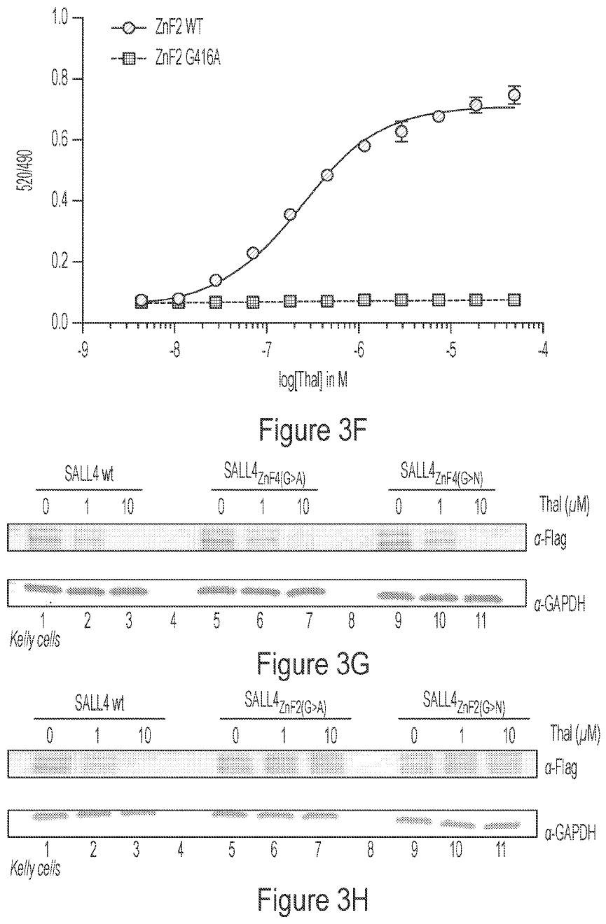

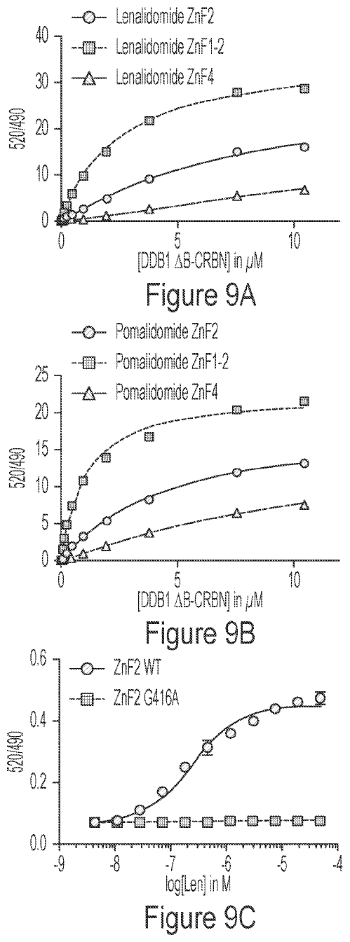

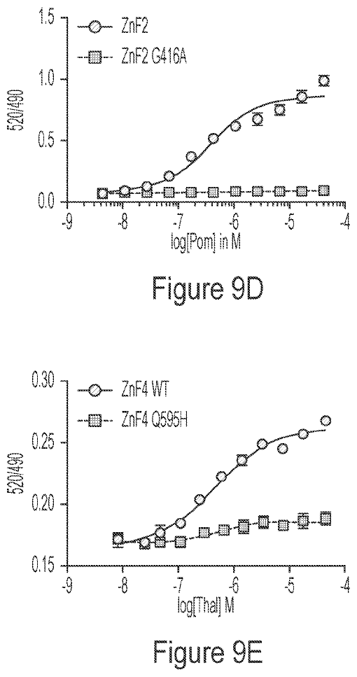

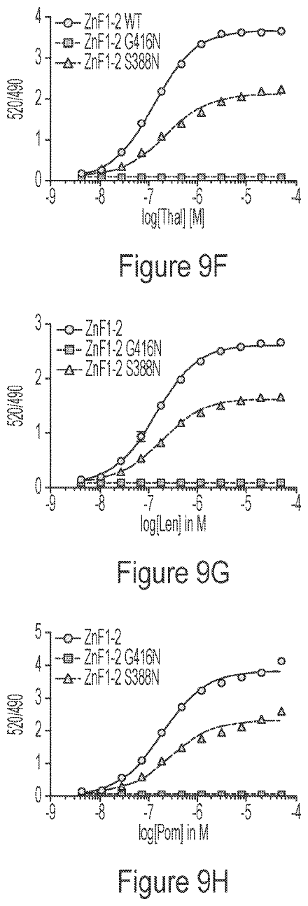

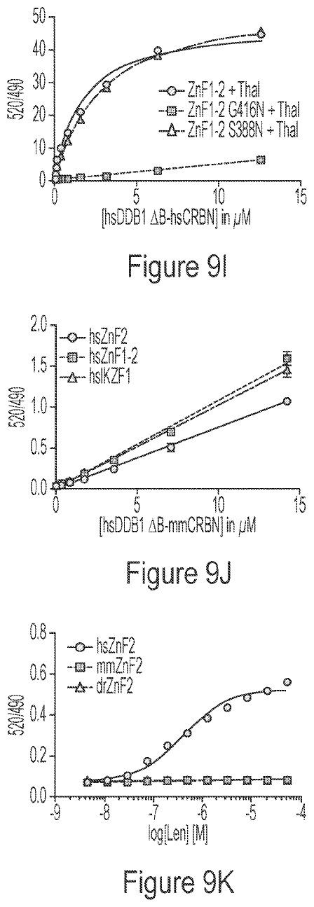

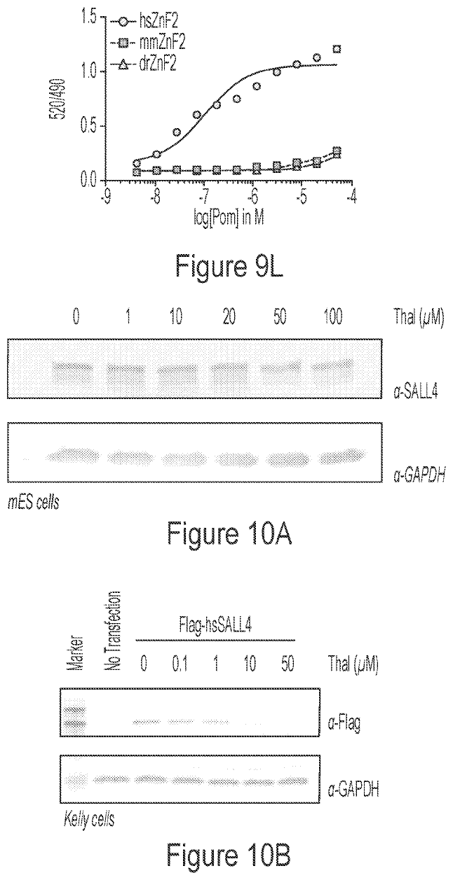

In Vitro binding assays confirm IMiD-dependent binding of SALL4 to CRL4^{CRBN} Bonafide targets of IMiD-induced degradation typically bind to CRBN (the substrate-recognition domain of the E3 ligase) in vitro in a compound-dependent manner. Thus, it was sought to test whether SALL4 binds to CRBN and to map the ZnF domain required for binding using purified recombinant proteins. Based on conserved features among IMiD sensitive ZnF domains (FIG. 3A, C-x(2)-C-G motif within the canonical C₂H₂ zinc finger motif), the second (SALL4_{ZnF2}) and fourth (SALL4_{ZnF4}) ZnF domains of SALL4 (aa 410-433, and aa 594-616, respectively) were identified as candidate degrons for IMiD-induced binding. These ZnF domains were expressed, purified, biotinylated, and subjected to in vitro CRBN binding assays (An et al., 2017; Petzold et al., 2016). Dose dependent binding was observed between SALL4_{ZnF2} or SALL4_{ZnF4} and CRBN similar to that described for IKZF1/3 and ZFP91, albeit with reduced apparent affinity for SALL4_{ZnF4} (FIGS. 3B and 3C) (Petzold et al., 2016). To estimate apparent affinities (K_{D(app)}) bodipy-FL labelled DDB1ΔB-CRBN was titrated to biotinylated SALL4_{ZnF2}, or SALL4_{ZnF4} at 100 nM with saturating concentrations of IMiDs (50 μM) and measured the affinity by TR-FRET (FIG. 3D and FIGS. 9A-9B), which confirmed the weak affinity of SALL4_{ZnF4}. However, it was noticed that a construct spanning ZnF1 and ZnF2 of SALL4 (SALL4_{ZnF1-2}) exhibited even tighter binding to CRBN (FIG. 3D and FIGS. 9A-9B) and enhanced dose dependent complex formation in TR-FRET (FIG. 3E). These findings suggest that multiple zinc finger domains of SALL4 contribute to binding, and may result in a multivalent recruitment to CRBN in vivo. However, the strength of the interaction with ZnF4 is unlikely to be sufficient for degradation in cells, and moreover, the rank order of Pom >Thal>>Len in binding observed with ZnF2 is in accordance with the cellular potency in degradation of SALL4, suggesting that ZnF2 is the critical ZnF domain for SALL4 degradation. The specificity of the SALL4_{ZnF2} interaction was confirmed by introducing a point mutation to glycine 416 (G416), the residue critical for IMiD-dependent binding to CRBN (Petzold et al., 2016). Mutations to alanine (G416A) rendered SALL4_{ZnF2} resistant to IMiD-dependent binding to CRBN (FIG. 3F and FIGS. 9C-9D). Mutating glutamine 595 (Q595) in SALL4_{ZnF4}, another residue previously shown to be critical for IMiD-dependent CRBN binding in the ZnF domains of IKZF1/3, impaired IMiD-dependent binding (FIG. 9E), confirming the specificity of the interaction despite the weak binding affinity. Since increased affinity of the tandem-ZnF construct SALL4_{ZnF1-2} was observed compared to the single SALL4_{ZnF2}, it was sought to test whether ZnF1 was sufficient for binding. The G416N mutation was introduced in ZnF2 or a S388N mutation in ZnF1 into the SALL4_{ZnF1-2} construct (S388 is the ZnF1 sequence equivalent of ZnF2 G416; ZnF1-2: C-x-x-C-S/G) and performed CRBN binding assays. G416N, but not S388N, fully abrogated IMiD-dependent binding of SALL4_{ZnF1-2} to CRBN (FIGS. 9F-9I) confirming the strict dependence on the ZnF2 interaction with CRBN. To test whether the second zinc finger of SALL4 is critical for IMiD-induced degradation in cells, G416A and G416N mutations were introduced into Flag-tagged full length SALL4. When expressed in Kelly cells, the parental wild type Flag-SALL4 was readily degraded by thalidomide treatment (FIG. 3G). Similarly, Flag-tagged SALL4 with G600A or G600N mutations in ZnF4 were also shown to be readily degraded with thalidomide treatment, suggesting that SALL4_{ZnF4} is dispensable for binding and subsequent degradation (FIG. 3G). Finally, the two conservative mutations in ZnF2 (G416A or G416N), both known to specifically disrupt binding to CRBN while maintaining the overall zinc finger fold (Petzold et al., 2016), rendered SALL4 stable under these treatment conditions, demonstrating that SALL4_{ZnF2} is necessary for CRL4^{CRBN}mediated degradation of SALL4 in cells (FIG. 3H). In vitro ubiquitination assays further confirm that SALL4_{ZnF1-2} is ubiquitinated by CRL4^{CRBN} in an IMiD-dependent fashion (FIG. 3I). Together, the cellular and biochemical data establish SALL4 as a bonafide IMiD-dependent target of CRL4^{CRBN}, and demonstrate that the second zinc finger is necessary for IMiD-dependent degradation, while the tandem array of ZnF1-2 further strengthens the interaction in vitro.

### Species Specific Teratogenicity is a Result of Genetic Differences in Both CRBN and SALL4

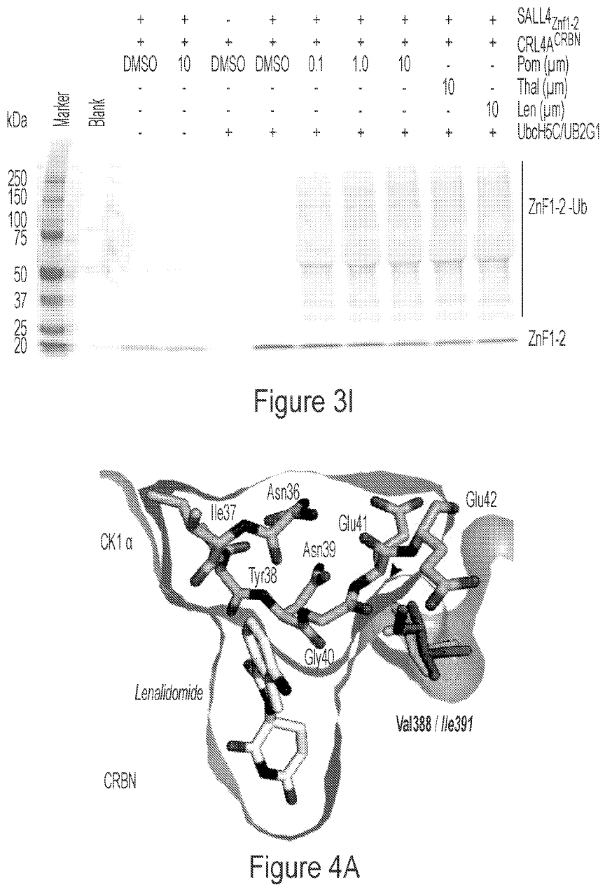

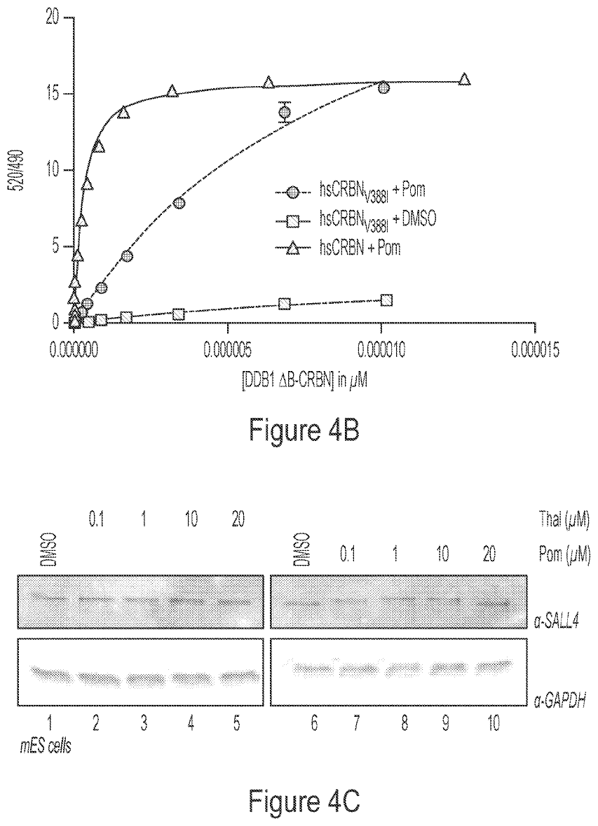

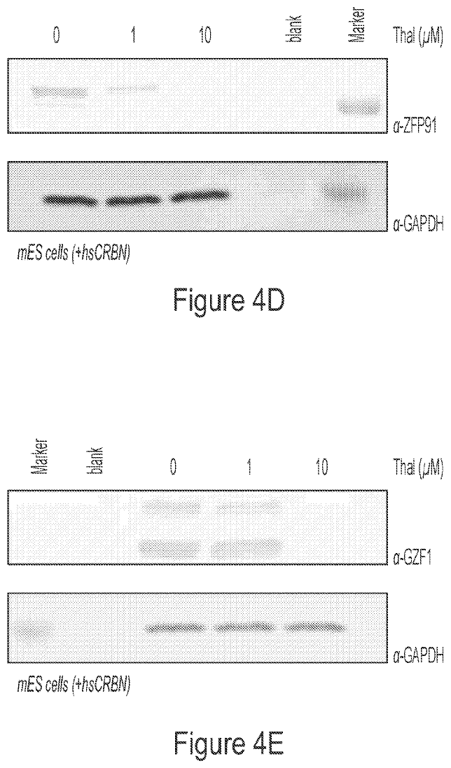

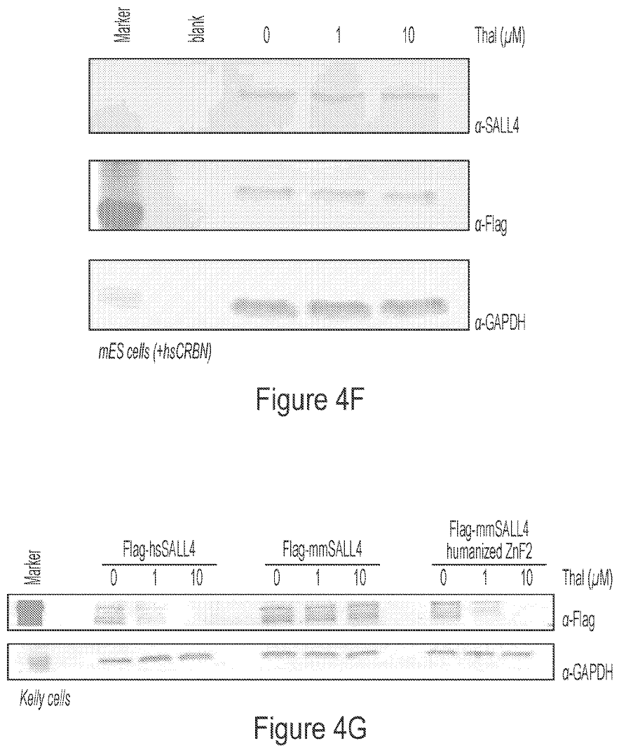

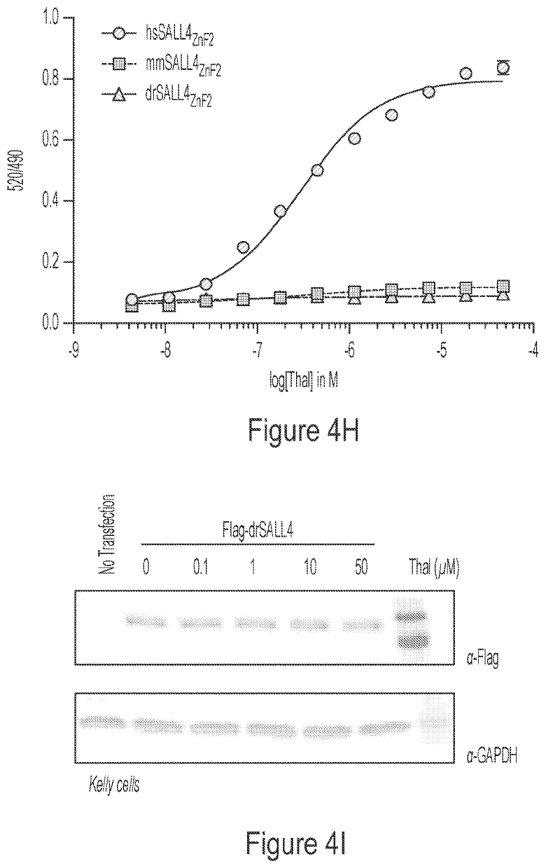

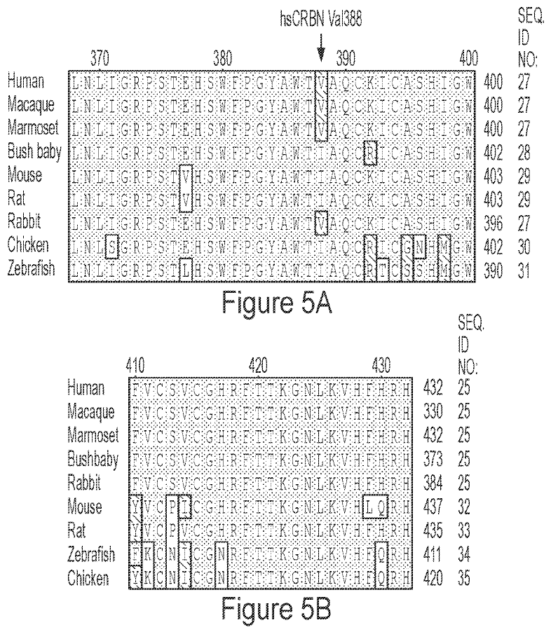

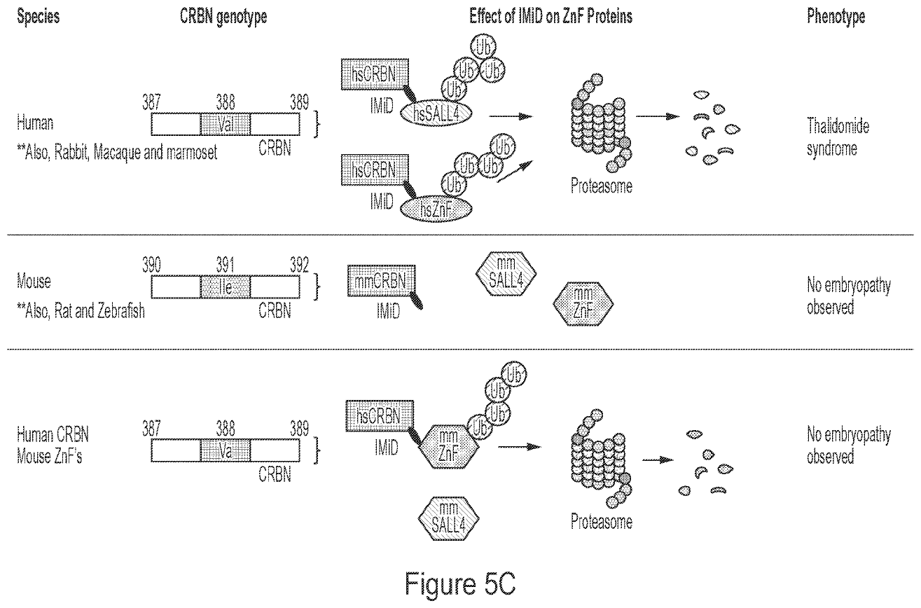

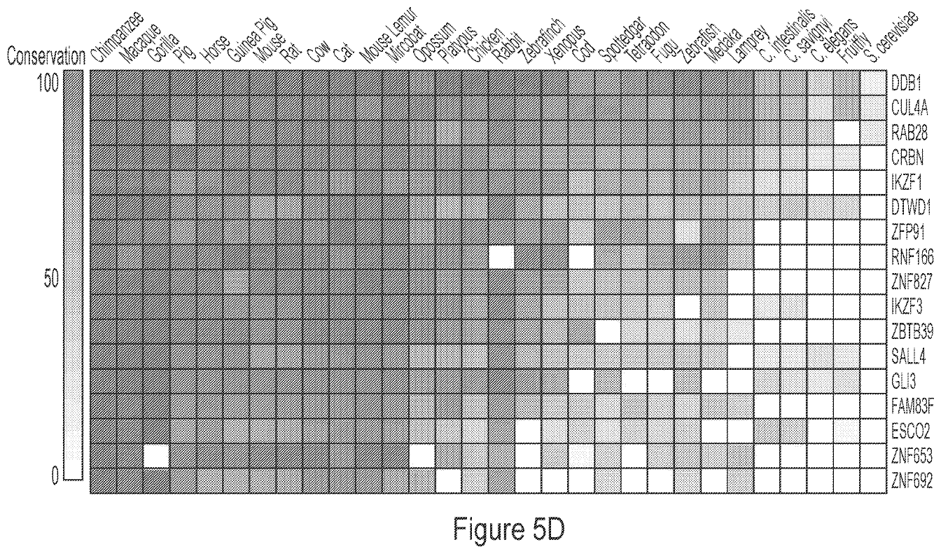

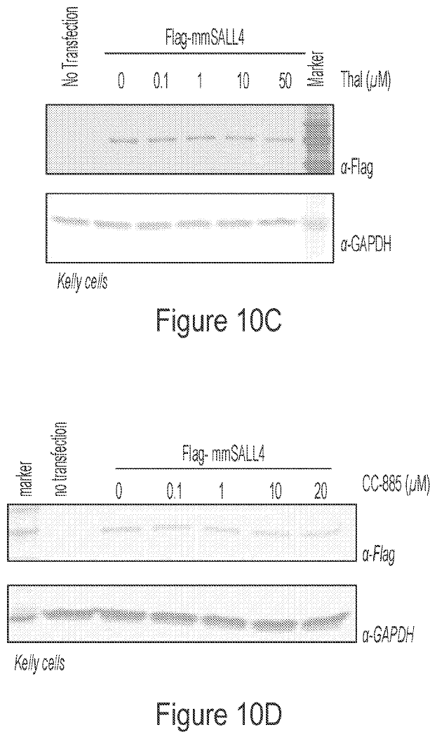

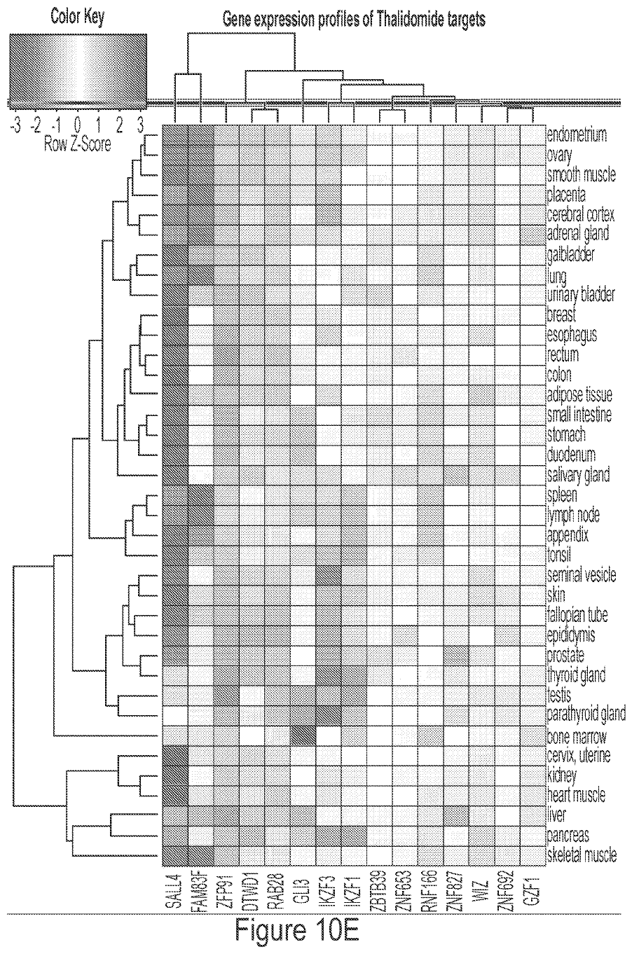

One characteristic feature of IMiD phenotypes is the absence of defining limb deformities following administration to pregnant rodents, which contributed to the initial approval by regulatory agencies in Europe. In contrast, many non-human primates exhibit phenotypes that mimic the human syndrome (Neubert et al., 1988; Smith et al., 1965; Vickers, 1967). These remarkable species specific phenotypes have historically complicated studies of thalidomide embryopathies, and suggest a genetic difference between these species that would abrogate the detrimental effects of thalidomide. Mouse Carbon harbors a critical polymorphism (FIGS. 4A, 4B and FIGS. 5A-5D) that prevents IMiD-dependent degradation of ZnF substrates and CSNK1A1 (Kronke et al., 2015), which could explain the absence of a SALL4 dependent phenotype in mice. Mouse and rat (both insensitive to thalidomide embryopathies) harbor an isoleucine at CRBN position 388 (residue 388 refers to the human CRBN sequence), in contrast, sensitive primates have a valine in position 388 that is necessary for CRL4^{CRBN} to bind, ubiquitinate, and subsequently degrade ZnF substrates (FIGS. 4A, 4B and FIG. 5A). Consistent with this concept, treatment of mouse embryonic stem cells (mESC) with increasing concentrations of thalidomide or pomalidomide does not promote degradation of mmSALL4 (FIG. 4C and FIG. 10A) and introducing a V388I mutation in hsCRBN renders the protein less effective to bind to SALL4 in vitro (FIG. 4B). It was thus asked whether ectopic expression of hsCRBN in mouse cells would lead to IMiD-induced degradation of mmSALL4, similar to what had been observed for CSNK1A1, and could hence render mice sensitive to IMiD-induced birth defects. Expression of hsCRBN in mouse cells, while sensitizing cells to degradation of IMiD targets such as mmIKZF1/3, mmCSNK1A1 (Kronke et al., 2015), mmZFP91 or mmGZF1 (FIGS. 4D and 4E), does not result in degradation of mmSALL4 (FIG. 4F). To test whether a fully human CRBN in a human cell background would be sufficient to induce SALL4 degradation, hsSALL4, or mmSALL4 was introduced into human cells (Kelly cells) and found that while ectopically expressed hsSALL4 is readily degraded upon IMiD treatment, mmSALL4 is unaffected even at arbitrarily high doses of IMiDs (FIG. 4G and FIGS. 10B-10C). Sequence analysis reveals that mice and zebrafish have critical mutations in the ZnF2 domain of SALL4 (FIG. 5B), which abrogate binding to hsCRBN in vitro (FIG. 4H), and render mmSALL4 and drSALL4 insensitive to IMiD mediated degradation in cells (FIGS. 4G, 4I and FIG. 10C). In line with these findings, mice harboring a homozygous CRBN I391V knock-in allele, despite exhibiting degradation of mmIKZF1/3, mmZFP91, and mmCSNK1A1 (Fink et al., submitted manuscript), show increased miscarriage upon IMiD treatment compared to control mice, however, do not exhibit IMiD-induced embryopathies resembling the human phenotype (Fink et al., submitted manuscript). It was next sought to test whether exchange of the mmSALL4 ZnF2 domain for the hsSALL4 ZnF2 domain would be sufficient to enable mmSALL4 degradation in a human cell line (Kelly cells). Strikingly, through the five base substitutions required to ‘humanize’ the mmSALL4 ZnF2 domain, thalidomide-mediated mouse SALL4 degradation was induced in a human cell line (FIG. 4G).

The observation, that SALL4 degradation depends on both the sequence of SALL4 (zinc finger 2 differs between human and rodents), and the sequence of CRBN, supports a genetic cause for the species specific effects, and highlights the complexities of modelling teratogenic adverse effects of IMiDs in murine and other animal models (Sakaki-Yumoto et al., 2006) (FIGS. 5A-5C). Noteworthy, the only non-human primate known to be insensitive to thalidomide induced embryopathies, the greater bush baby, also harbors an isoleucine in the critical CRBN V388 position (Butler, 1977), while all sensitive non-human primates and rabbits harbor the conserved valine (FIG. 5A). It is thus shown that species can be rendered resistant by either mutations in CRBN, SALL4 or both, and hence the data suggests that thalidomide embryopathy is primarily a human disease (with some non-human primates, and rabbits more closely resembling the phenotypes), and thus explain the historic observation that modelling thalidomide embryopathies in animals is challenging. It is noted that zebrafish and chicken both contain an Ile in the V388 position, however, were reported to exhibit defects to limb/fin formation upon exposure to thalidomide or knock-down of Crbn (Eichner et al., 2016; Ito et al., 2010), partially resembling thalidomide induced defects. These findings are in contrast with the observations in higher eukaryotes, as Crbn knock-out mice have been reported to exhibit normal morphology (Lee et al., 2013), and children harboring a homozygous C391R mutation in CRBN (C391 is a structural cysteine coordinating the zinc in the thalidomide binding domain of CRBN and any protein from a C391R cDNA was failed to be produced), a loss of function mutation, were born without characteristic birth defects but exhibited severe neurological defects (Sheereen et al., 2017). Whether the phenotypes in zebrafish and chicken are a result of species-specific downstream pathways or the high dose (400 μM) and direct application of thalidomide to the limb buds (Ito et al., 2010), which both could result in off-target effects, remains to be shown. The plasma concentration of thalidomide in humans will, however, unlikely exceed 10 μM (Bai et al., 2013; Dahut et al., 2009), a concentration that results in effective degradation of SALL4, but is forty times below the dose found to be teratogenic in chicken and zebrafish embryos. While degradation of mmSALL4 or drSALL4 is not observed upon high dose exposure, it cannot be ruled out that such high doses will induce degradation of other ZnF targets in zebrafish or chicken, which could potentially result in the observed phenotypes. In fact, it is shown that IMiDs lead to degradation of multiple ZnF transcription factors, a class of proteins known to evolve very rapidly (Schmitges et al., 2016), and it is likely that IMiDs will exhibit species specific effects. Sequence analysis shows that IMiD-dependent ZnF targets such as SALL4, ZNF653, ZNF692, or ZBTB39 as well as other known genetic causes of limb defects in ZnF transcription factors, such as ESCO2, are highly divergent even in higher eukaryotes (FIG. 5D).

### Discussion

It is shown that thalidomide, lenalidomide and pomalidomide all induce degradation of SALL4, which has been causatively linked to the most characteristic and common birth defects of the limbs and inner organs by human genetics. While other targets of thalidomide, such as CSNK1A1 for lenalidomide or GZF1, ZBTB39 for pomalidomide may contribute to the pleiotropic developmental conditions observed upon thalidomide exposure, SALL4 is consistently degraded across all IMiDs and human genetics associate heterozygous loss of SALL4 with human developmental syndromes that largely phenocopy thalidomide syndrome. Moreover, from the targets degraded across IMiDs, IKZF1/3 have been shown to be non-causative for birth defects, RNF166 is a ubiquitin ligase involved in autophagy (Heath et al., 2016), and ZNF692 knock-out mice do not exhibit a teratogenic phenotype [International Mouse Phenotyping Consortium]. While only genetic studies in non-human primates or rabbits can provide the ultimate molecular role of SALL4 and other targets in thalidomide embryopathies, the known functions of SALL4 are consistent with a potential role in thalidomide embryopathies.

The polypharmacology of IMiDs (most notably pomalidomide), together with the size and rapid evolution of the C₂H₂ family of zinc finger transcription factors (FIG. 5D), which results in most C₂H₂ zinc finger transcription factors being highly species specific (Najafabadi et al., 2015; Schmitges et al., 2016), help to explain the pleiotropic effects of IMiDs, which still remain largely understudied. Thalidomide embryopathies thus represent a case in which animal studies fall short, and it is likely that the clinical features of IMiD efficacy as well as adverse effects, are a result of induced degradation of multiple C₂H₂ zinc finger transcription factors. For example, some degree of degradation is seen for GZF1, another C₂H₂ transcription factor, while GZF1 is unlikely to cause the defining birth defects of thalidomide, mutations in GZF1 have been associated with joint laxity and short stature, which are both also found in thalidomide affected children (Patel et al., 2017). It is also noticed that CRBN expression levels influence the efficacy of IMiDs in inducing protein degradation, and it is conceivable that these contribute to a certain degree of tissue selectivity of IMiD effects, which for example, could increase the therapeutic index in MM since hematopoietic lineages tend to have high levels of CRBN.

Thalidomide teratogenicity was a severe and widespread public health tragedy, affecting more than 10,000 individuals, and the aftermath has shaped many of the current drug regulatory procedures. The findings that thalidomide and its derivatives induce degradation of SALL4 provide a direct link to genetic disorders of SALL4 deficiency, which phenocopy many of the teratogenic effects of thalidomide. While other effects of thalidomide, such as anti-angiogenic properties may contribute to birth defects, degradation of SALL4 will likely contribute to birth defects. These findings can inform the development of new compounds that induce CRBN-dependent degradation of disease-relevant proteins but avoid degradation of developmental transcription factors such as SALL4, and thus have the potential for therapeutic efficacy without the risk of teratogenicity, a defining feature of this class of drugs. This is further relevant to the development of thalidomide-derived bifunctional small molecule degraders (commonly referred to as PROTACs) (Raina and Crews, 2017), since it is shown that IMiD based PROTACs (and novel IMiD derivatives such as CC-220) can be effective inducers of ZnF targets including SALL4 degradation (FIG. 6C). Lastly, the surprising expansion in substrate repertoire for pomalidomide, suggest that IMiDs exhibit a large degree of polypharmacology contributing to both efficacy and adverse effects. Transcription factors, and specifically C₂H₂ zinc fingers are highly divergent between species, and hence IMiDs and related compounds will likely exhibit species specific effects by virtue of their mode of action. In turn, the discovery that IMiDs target an unanticipated large set of C₂H₂ zinc finger proteins with significant differences between thalidomide, lenalidomide, pomalidomide and CC-220, suggests that this chemical scaffold holds the potential to target one of the largest families of human transcription factors.

### Materials and Methods

TABLE 2
Key resources

| Reagent type (species) | | | | Additional |
|---|---|---|---|---|
| or resource | Designation | Source or reference | Identifiers | information |
| gene (H. sapiens) | CRBN | Fischer et al., Nature 2014 | Gene ID: 51185 | |
| gene (M. musculus) | CRBN | Dr. Ben Ebert (Brigham and | Gene ID: 58799 | |
| | | Womens Hospital, Dana | | |
| | | Farber Cancer Institute) | | |
| gene (H. sapiens) | SALL4 | IDT | Gene ID: 57167 | |
| gene (H. sapiens) | DDB1ΔB | Petzold et al., Nature 2016 | Gene ID: 1642 | |
| gene (M. musculus) | SALL4 | IDT | Gene ID: 99377 | |
| gene (D. rerio) | SALL4 | IDT | Gene ID: 572527 | |
| cell line (H. sapiens) | H9 hES cells | Dr. Wade Harper (Harvard | RRID: CVCL_9773 | |
| | | Medical School) | | |
| cell line (H. sapiens) | Kelly Cells | Dr. Nathanael Gray (Dana | RRID: CVCL_2092 | |
| | | Farber Cancer Institute, | | |
| | | Harvard Medical School) | | |
| cell line (H. sapiens) | SK-N-DZ cells | ATCC | RRID: CVCL_1701; | |
| | | | CRL-2149 | |
| cell line (H. sapiens) | MM1s cells | ATCC | RRID: CVCL_8792; | |
| | | | CRL-2974 | |
| cell line (H. sapiens) | H661 cells | ATCC | RRID: CVCL_1577; | |
| | | | HTB-183 | |
| cell line (H. sapiens) | HEK293T cells | ATCC | RRID: CVCL_0063; | |
| | | | CRL-3216 | |
| cell line (M. musculus) | TC1 mESC | Dr. Richard Gregory | RRID: CVCL_M350 | |
| | cells | (Boston Childrens Hospital, | | |
| | | Harvard Medical School) | | |
| cell line (T. ni) | High Five | Thermo Fisher Scientific | RRID: CVCL_C190; | |
| | insect cells | | B85502 | |
| chemical compound, drug | Thalidomide | MedChemExpress | HY-14658 | |
| chemical compound, drug | Lenalidomide | MedChemExpress | HY-A0003 | |
| chemical compound, drug | Pomalidomide | MedChemExpress | HY-10984 | |
| chemical compound, drug | CC-220 | MedChemExpress | HY-101291 | |
| chemical compound, drug | CC-885 | Cayman chemical | 19966 | |
| chemical compound, drug | dBET57 | Nowak et al., Nat Chem | | |
| | | Biol 2018 | | |
| chemical compound, drug | Bortezomib | MedChemExpress | HY-10227 | |
| chemical compound, drug | MLN4924 | MedChemExpress | HY-70062 | |
| chemical compound, drug | MLN7243 | Active Biochem | A1384 | |
| recombinant DNA | pCDH-MSCV (PGK | Dr. Ben Ebert (Brigham and | | |
| reagent | promoter plasmid) | Womens Hospital, Dana | | |
| | | Farber Cancer Institute) | | |
| recombinant DNA | pNTM (CMV | Dr. Nicolas Thomä, FMI, | | |
| reagent | promoter plasmid) | Switzerland | | |
| recombinant DNA | pAC8 (Polyhedrin | Dr. Nicolas Thomä, FMI, | | |
| reagent | promoter plasmid) | Switzerland | | |
| peptide, recombinant | hsHis6-3C- | Nowak et al., Nat Chem | | |
| protein | Spy-CRBN | Biol 2018 | | |
| peptide, recombinant | hsHis6-3C-Spy- | This study | | |
| protein | CRBN_V388I | | | |
| peptide, recombinant | hsStrep-BirA- | This study | | |
| protein | SALL4 (590-618) | | | |
| peptide, recombinant | hsStrep-BirA- | This study | | |
| protein | SALL4_Q595H | | | |
| | (590-618) | | | |
| peptide, recombinant | hsStrep-BirA- | This study | | |
| protein | SALL4 (378-438) | | | |
| peptide, recombinant | hsStrep-BirA- | This study | | |
| protein | SALL4 (402-436) | | | |
| peptide, recombinant | mmStrep-BirA- | This study | | |
| protein | SALL4 (593-627) | | | |
| peptide, recombinant | drStrep-BirA- | This study | | |
| protein | SALL4 (583-617) | | | |
| peptide, recombinant | SpyCatcher | Nowak et al., Nat Chem | | |
| protein | S50C | Biol 2018 | | |
| peptide, recombinant | His-hsDDB1(1- | Fischer et al., Cell 2011 | | |
| protein | 1140)-His- | | | |
| | hsCUL4A(38-759)- | | | |
| | His-mmRBX1(12- | | | |
| | 108) (CRL4-CRBN) | | | |
| peptide, recombinant | Ubiquitin | Boston Biochem | U-100H | |
| protein | | | | |
| peptide, recombinant | His-E1 | Boston Biochem | E-304 | |
| protein | | | | |
| peptide, recombinant | UBE2G1 | Boston Biochem | E2-700 | |
| protein | | | | |
| peptide, recombinant | UbcH5c | Boston Biochem | E2-627 | |
| protein | | | | |
| antibody | Mouse anti- | abcam | RRID: AB_2183366; | WB (1:250) |
| | SALL4 | | ab57577 | |
| antibody | Rabbit anti- | abcam | RRID: AB_777810; | WB (1:250) |
| | SALL4-chip | | ab29112 | |
| | grade | | | |
| antibody | Rabbit anti- | Sigma Aldrich | RRID: AB_2677903; | WB (1:500) |
| | DTWD1 | | HPA042214 | |
| antibody | Mouse anti- | Sigma Aldrich | RRID: AB_262044; | WB |
| | FLAG M2 | | F1804 | (1:1000) |
| antibody | Rabbit anti- | Novus Biologicals | RRID: AB_11037820; | WB (1:500) |
| | CRBN | | NBP1-91810 | |
| antibody | Rabbit anti- | Thermo Fisher Scientific | RRID: AB_2551727; | WB (1:500) |
| | GZF1 | | PA534375 | |
| antibody | Mouse anti- | Sigma Aldrich | RRID: AB_1078991; | WB |
| | GAPDH | | G8795 | (1:10,000) |
| antibody | IRDye680 | LiCor | RRID: AB_10953628; | WB |
| | Donkey anti- | | 92668072 | (1:10,000) |
| | mouse IgG | | | |
| antibody | IRDye800 | LiCor | RRID: AB_621843; | WB |
| | Goat anti- | | 92632211 | (1:10,000) |
| | rabbit | | | |
| antibody | Rabbit anti- | abcam | RRID: AB_1524455; | WB |
| | Strep-Tag II | | ab76949 | (1:10,000) |
| antibody | anti-Strep-Tag | Millipore | RRID: AB_10806716; | WB |
| | II HRP conjugate | | 71591 | (1:10,000) |
| antibody | anti-Mouse IgG | Cell Signalling | RRID: AB_330924; | WB |
| | HRP conjugate | | 7076 | (1:10,000) |
| other | Amersham ECL | GE healthcare | RPN2232 | |
| | prime western | | | |
| | blot reagent | | | |
| other | BODIPY-FL- | Thermo Fisher Scientific | B10250 | |
| | Maleimide | | | |
| other | Tb streptavidin | Invitrogen | LSPV3966 | |
| other | TMT 10-plex | Thermo Fisher Scientific | 90406 | |
| | labels | | | |
| other | Lipofectamine | Invitrogen | 11668019 | |
| | 2000 | | | |

### Compounds, Enzymes and Antibodies

Thalidomide (HY-14658, MedChemExpress), lenalidomide (HY-A0003, MedChemExpress), pomalidomide (HY-10984, MedChemExpress), CC-220 (HY-101291, MedChemExpress), CC-885 (19966, Cayman chemical), dBET57 (Nowak et al., 2018), bortezomib (HY-10227, MedChemExpress), MLN4924 (HY-70062, MedChemExpress) and MLN7243 (A1384, Active Biochem) were purchased from the indicated vendors and subjected to in house LC-MS for quality control.

HEK293T, SK-N-DZ, MM1s and H661 were purchased from ATCC and cultured according to ATCC instructions. H9 hESC, mESC and Kelly cells were kindly provided by the labs of J. Wade Harper (HMS), Richard I. Gregory (TCH/HMS) and Nathanael Gray (DFCI/HMS) respectively. Sequencing grade modified trypsin (V5111) was purchased from Promega (Promega, USA) and mass spectrometry grade lysyl endopeptidase from Wako (Wako Pure Chemicals, Japan). Primary and secondary antibodies used included, anti-SALL4 at 1:250 dilution (ab57577, abcam—found reactive for human SALL4), anti-SALL4 chip grade at 1:250 dilution (ab29112, abcam—found reactive for mouse Sall4), anti-DTWD1 1:500 (HPA042214, Sigma), anti-Flag 1:1000 (F1804, Sigma), anti-CRBN 1:500 (NBP1-91810, Novus Biologicals), anti-GZF1 at 1:500 (PA534375, Thermo Fisher Scientific), anti-GAPDH at 1:10,000 dilution (G8795, Sigma), IRDye680 Donkey anti-mouse at 1:10,000 dilution (926-68072, LiCor), IRDye800 Goat anti-rabbit at 1:10,000 dilution (926-32211, LiCor) and rabbit anti-Strep-Tag II antibody at 1:10,000 (ab76949, Abcam), anti-mouse IgG HRP-linked Antibody at 1:10,000 dilution (7076, Cell Signaling), Amersham ECL Prime Western Blotting Detection Reagent (RPN2232, GE).

### Cell Culture

HEK293T cells were cultured in DMEM supplemented with 10% dialyzed fetal bovine serum (FBS) and 2 mM L-Glutamine. SK-N-DZ cells were cultured in DMEM supplemented with 10% dialyzed FBS, 0.1 mM Non-Essential Amino Acids (NEAA) and 2 mM L-Glutamine. H661, MM1s and Kelly cells were cultured in RPMI1640 supplemented with 10% dialyzed FBS. H9 hESC cells were cultured in Essential 8 (Gibco) media on Matrigel-coated nunc tissue culture plates. TC1 mouse embryonic stem cells (mESCs) were adapted to gelatin cultures and fed with KO-DMEM (Gibco) supplemented with 15% stem cell-qualified fetal bovine serum (FBS, Gemini), 2 mM L-glutamine (Gibco), 20 mM HEPES (Gibco), 1 mM sodium pyruvate (Gibco), 0.1 mM of each non-essential amino acids (Gibco), 0.1 mM 2-mercaptoethanol (Sigma), 104 U mL⁻¹ penicillin/streptomycin (Gibco), and 103 U mL⁻¹ mLIF (Gemini).

Cell lines were acquired from sources provided in the key resource table. All cell lines are routinely authenticated using ATCC STR service, and are tested for mycoplasma contamination on a monthly basis. All cell lines used for experiments tested negative.

### Western Blot

Cells were treated with compounds as indicated and incubated for 24 hours, or as indicated. Samples were run on 4-20%, AnyKD or 10% (in-vitro ubiqutination assay) SDS-PAGE Gels (Bio-rad), and transferred to PVDF membranes using the iBlot 2.0 dry blotting system (Thermo Fisher Scientific). Membranes were blocked with LiCor blocking solution (LiCor), and incubated with primary antibodies overnight, followed by three washes in LiCor blocking solution and incubation with secondary antibodies for one hour in the dark. After three final washes, the membranes were imaged on a LiCor fluorescent imaging station (LiCor). When Anti-mouse IgG, HRP Antibody was used, after three washes, the membranes were incubated with Amersham ECL Prime Western Blotting Detection Reagent for 1 minute and subjected to imaging by Amersham Imager 600 (GE).

### Q5 Mutagenesis and Transient Transfection

hsCRBN, hsSALL4, mmSALL4 and drSALL4 were PCR amplified and cloned into a pNTM-Flag based vector. Mutagenesis was performed using the Q5 site-directed mutagenesis kit (NEB, USA) with primers designed using the BaseChanger web server (http://nebasechanger.neb.com/).

Primer sets used for Q5 mutagenesis are:

hsSALL4-S388N
(SEQ ID NO: 3)
Fwd 5′-3′: AAGTACTGTAaCAAGGTTTTTG
(SEQ ID NO: 4)
Rev 5′-3′: ACACTTGTGCTTGTAGAG
hsSALL4-G416A
(SEQ ID NO: 5)
Fwd 5′-3′: TCTGTCTGTGcTCATCGCTTCAC
(SEQ ID NO: 6)
Rev 5′-3′: GCACACGAAGGGTCTCTC
hsSALL4-G416N
(SEQ ID NO: 7)
Fwd 5′-3′: CTCTGTCTGTaaTCATCGCTTCACCAC
(SEQ ID NO: 8)
Rev 5′-3′: CACACGAAGGGTCTCTCT
hsSALL4-G600A
(SEQ ID NO: 9)
Fwd 5′-3′: AAGATCTGTGcCCGAGCCTTTTC
(SEQ ID NO: 10)
Rev 5′-3′: ACACTGGAACGGTCTCTC
hsSALL4-G600N
(SEQ ID NO: 11)
Fwd 5′-3′: TAAGATCTGTaaCCGAGCCTTTTCTAC
(SEQ ID NO: 12)
Rev 5′-3′: CACTGGAACGGTCTCTCC
Humanizing mmSALL4-Y415F, P418S,
I419V, L430F, Q435H
(SEQ ID NO: 13)
Fwd 5′-3′: AGGGCAATCTCAAGGTCCACTTtCAcC
GACACCCTCAGGTGAAGGCAAACCCCC
(SEQ ID NO: 14)
Rev 5′-3′:
TGGTGGTGAAGCGGTGACCACAGAcAGaGCACACGaAA
GGTCTCTCTCCGGTGTG

For transient transfection, 0.2 million cells were seeded per well in a 12 well plate on day one. On day two, cells were transfected with 200-300 ng of plasmid (pNTM-Flag containing gene of interest) using 2 μL of lipofectamine 2000 transfection reagent (Invitrogen). On day three, desired concentration of IMiD was added to each well and cells were harvested after 24 hours for western blot analysis using the protocol described above.

Constructs and Protein Purification _{His6}DDB1ΔB (Petzold et al., 2016), _{His6-3C-Spy}hsCRBN, _{His6-3C-Spy}hsCRBN^{V388I}, _{Strep-Bir}_{_{A}}hsSALL4₅₉₀₋₆₁₈ (ZnF4), _{Strep-Bir}_{_{A}}hSSALL4^{Q595H}₅₉₀₋₆₁₈ (ZnF4), _{Strep-Bir}_{_{A}}hsSALL4₃₇₈₋₄₃₈ (ZnF1-2), _{Strep-Bir}_{_{A}}hsSALL4₄₀₂₋₄₃₆ (ZnF2), _{Strep-Bir}_{_{A}}mmSALL4₅₉₃₋₆₂₇ (ZnF4), _{Strep-Bir}_{_{A}}drSALL4₅₈₃₋₆₁₇ (ZnF2) were subcloned into pAC-derived vectors or BigBac vector for _{His}hsDDB1_{1-1140-His}hsCUL4A_{38-759-Hhis}mmRBX1₁₂₋₁₀₈ (CRL4^{CRBN}). Mutant _{Strep-Bir}_{_{A}}hsSALL4₃₇₈₋₄₃₈ (ZnFT-2) and _{Strep-Bir}_{_{A}}hsSALL4₄₀₂₋₄₃₆ (ZnF2) constructs were derived from these constructs using Q5 mutagenesis (NEB, USA). Recombinant proteins expressed in Trichoplusia ni High Five insect cells (Thermo Fisher Scientific) using the baculovirus expression system (Invitrogen). For purification of DDB1ΔB-CRBN_{SpyBodipyFL} or CRL4^{CRBN} cells were resuspended in buffer containing 50 mM tris(hydroxymethyl)aminomethane hydrochloride (Tris-HCl) pH 8.0, 200 mM NaCl, 1 mM tris(2-carboxyethyl)phosphine (TCEP), 1 mM phenylmethylsulfonyl fluoride (PMSF), 1× protease inhibitor cocktail (Sigma) and lysed by sonication. Cells expressing variations of _{Strep-Bir}_{_{A}}SALL4 were lysed in the presence of 50 mM Tris-HCl pH 8.0, 500 mM NaCl, 1 mM TCEP, 1 mM PMSF and 1× protease inhibitor cocktail (Sigma). Following ultracentrifugation, the soluble fraction was passed over appropriate affinity resin Ni Sepharose 6 Fast Flow affinity resin (GE Healthcare) or Strep-Tactin Sepharose XT (IBA), and eluted with 50 mM Tris-HCl pH 8.0, 200 mM NaCl, 1 mM TCEP, 100 mM imidazole (Fischer Chemical) for His₆-tagged proteins or 50 mM Tris-HCl pH 8.0, 500 mM NaCl, 1 mM TCEP, 50 mM D-biotin (IBA) for Strep tagged proteins. Affinity-purified proteins were either further purified via ion exchange chromatography (Poros 50HQ) and subjected to size exclusion chromatography (SEC200 HiLoad™ 16/60, GE) (_{His6}DDB1ΔB-_{His6-3C-Spy}CRBN or CRL4^{CRBN}) or biotinylated over-night, concentrated and directly loaded on the size exclusion chromatography (ENRich SEC70 10/300, Bio-rad) in 50 mM HEPES pH 7.4, 200 mM NaCl and 1 mM TCEP. Biotinylation of _{Strep-Bir}_{_{A}}SALL4 constructs was performed as previously described (Cavadini et al., 2016).

The protein-containing fractions were concentrated using ultrafiltration (Millipore), flash frozen in liquid nitrogen, and stored at −80° C. or directly covalently labeled with BODIPY-FL-SpyCatcher_{S50C} as described below.

### Spycatcher S50C Mutant

Spycatcher (Zakeri et al., 2012) containing a Ser50Cys mutation was obtained as synthetic dsDNA fragment from IDT (Integrated DNA technologies) and subcloned as GST-TEV fusion protein in a pET-Duet derived vector. Spycatcher S50C was expressed in BL21 DE3 and cells were lysed in the presence of 50 mM Tris-HCl pH 8.0, 200 mM NaCl, 1 mM TCEP and 1 mM PMSF. Following ultracentrifugation, the soluble fraction was passed over Glutathione Sepharose 4B (GE Healthcare) and eluted with wash buffer (50 mM Tris-HCl pH 8.0, 200 mM NaCl, 1 mM TCEP) supplemented with 10 mM glutathione (Fischer BioReagents). The affinity-purified protein was subjected to size exclusion chromatography, concentrated and flash frozen in liquid nitrogen.

### In-Vitro Ubiquitination Assays

In vitro ubiquitination was performed by mixing biotinylated SALL4 ZnF1-2 at 0.6 μM, and CRL4^{CRBN} at 80 nM with a reaction mixture containing IMiDs at indicated concentrations or a DMSO control, E1 (UBA1, Boston Biochem) at 30 nM, E2 (UbcH5c, Boston Biochem and UBE2G1) at 1.0 μM each, ubiquitin (Ubiquitin, Boston Biochem) at 23 μM. Reactions were carried out in 50 mM Tris pH 7.5, 30 mM NaCl, 5 mM MgCl₂, 0.2 mM CaCl₂), 2.5 mM ATP, 1 mM DTT, 0.1% Triton X-100 and 2.0 mg mL⁻¹ BSA, incubated for 60 minutes at 30° C. and analyzed by western blot using rabbit anti-Strep-Tag II antibody at 1:10,000 (ab76949, Abcam) as described above.

### Lentiviral Infection of mES Cells

TC1 mES cells were transduced with a pCDH-MSCV-based lentiviral vector expressing hsCRBN, GFP and the puromycin resistance gene. Infection was performed after 24 hours in culture in a 6-well 0.2% gelatin coated plate using standard infection protocol in the presence of 2 μg mL⁻¹ polybrene (hexadimethrine bromide, Sigma). 72 hours after transduction the cells were subjected to two rounds of puromycin selection (5 μg mL⁻¹) to form mES cells stably expression hsCRBN, which were confirmed to be >90% GFP positive under fluorescent microscope.

Labeling of Spycatcher with BODIPY-FL-Maleimide

Purified Spycatcher_{S50C} protein was incubated with DTT (8 mM) at 4° C. for 1 hour. DTT was removed using a ENRich SEC650 10/300 (Bio-rad) size exclusion column in a buffer containing 50 mM Tris pH 7.5 and 150 mM NaCl, 0.1 mM TCEP. BODIPY-FL-maleimide (Thermo Fisher Scientific) was dissolved in 100% DMSO and mixed with Spycatcher_{S50C} to achieve 2.5 molar excess of BODIPY-FL-maleimide. SpyCatcher_{S50C} labeling was carried out at room temperature (RT) for 3 hours and stored overnight at 4° C. Labeled Spycatcher_{S50C} was purified on an ENRich SEC650 10/300 (Bio-rad) size exclusion column in 50 mM Tris pH 7.5, 150 mM NaCl, 0.25 mM TCEP and 10% (v/v) glycerol, concentrated by ultrafiltration (Millipore), flash frozen (˜40 μM) in liquid nitrogen and stored at −80° C.

### BODIPY-FL-Spycatcher Labeling of CRBN-DDB1ΔB

Purified _{His6}DDB1ΔB-_{His6-3C-Spy}CRBN constructs (WT and V388I) were incubated overnight at 4° C. with BODIPY-FL-maleimide labeled SpyCatcher_{S50C} protein at stoichiometric ratio. Protein was concentrated and loaded on the ENrich SEC 650 10/300 (Bio-rad) size exclusion column and the fluorescence monitored with absorption at 280 and 490 nm. Protein peak corresponding to the labeled protein was pooled, concentrated by ultrafiltration (Millipore), flash frozen in liquid nitrogen and stored at −80° C.

### Time-Resolved Fluorescence Resonance Energy Transfer (TR-FRET)

Compounds in binding assays were dispensed into a 384-well microplate (Coming, 4514) using the D300e Digital Dispenser (HP) normalized to 1% DMSO and containing 100 nM biotinylated Strep-Avi-SALL4 (WT or mutant, see Figure legends), 1 μM His₆-DDB1ΔB-His₆-CRBN_{BODIPY-Spycatcher} and 4 nM terbium-coupled streptavidin (Invitrogen) in a buffer containing 50 mM Tris pH 7.5, 100 mM NaCl, 1 mM TCEP, 0.1% Pluronic F-68 solution (Sigma). Before TR-FRET measurements were conducted, the reactions were incubated for 15 minutes at RT. After excitation of terbium fluorescence at 337 nm, emission at 490 nm (terbium) and 520 nm (BODIPY) were recorded with a 70 μs delay over 600 μs to reduce background fluorescence and the reaction was followed over 30×200 second cycles of each data point using a PHERAstar FS microplate reader (BMG Labtech). The TR-FRET signal of each data point was extracted by calculating the 520/490 nm ratios. Data from three independent measurements (n=3), each calculated as an average of 5 technical replicates per well per experiment, was plotted and the half maximal effective concentrations EC₅₀ values calculated using variable slope equation in GraphPad Prism 7. Apparent affinities were determined by titrating Bodipy-FL labelled DDB1ΔB-CRBN to biotinylated Strep-Avi-SALL4 (constructs as indicated) at 100 nM, and terbium-streptavidin at 4 nM. The resulting data were fitted as described previously (Petzold et al., 2016).

### Quantitative RT-PCR Analysis

H9 hES cells treated with 10 μM thalidomide or DMSO for 24 hours were subjected to gene expression analysis. RNA was isolated using the RNeasy Plus mini kit (Qiagen) and cDNA created by reverse transcription using ProtoScript II reverse transcriptase (NEB) following the manufacturer's instructions. The following primer sets from IDT were used with SYBR Green Master Mix (Applied Biosystems) to probe both GAPDH and total SALL4 levels:

(SEQ ID NO: 15)
SALL4tota1-F: GGTCCTCGAGCAGATCTTGT
(SEQ ID NO: 16)
SALL4tota1-R: GGCATCCAGAGACAGACCTT
(SEQ ID NO: 17)
GAPDH-F: GAAGGTGAAGGTCGGAGTC
(SEQ ID NO: 18)
GAPDH-R: GAAGATGGTGATGGGATTTC

Analysis was performed on a CFX Connect Real-Time PCR System (Bio-Rad) in a white 96-well PCR plate. Relative expression levels were calculated using the AACT method.

### Sample Preparation TMT LC-MS3 Mass Spectrometry

H9 hESC, Kelly, SK-N-DZ and MM1s cells were treated with DMSO, 1 μM pomalidomide, 5 μM lenalidomide or 10 μM thalidomide in biological triplicates (DMSO) or biological duplicates (pomalidomide, lenalidomide, thalidomide) for 5 hours and cells harvested by centrifugation. Lysis buffer (8 M Urea, 50 mM NaCl, 50 mM 4-(2hydroxyethyl)-1-piperazineethanesulfonic acid (EPPS) pH 8.5, 1× Roche protease inhibitor and 1× Roche PhosphoStop was added to the cell pellets and cells were homogenized by 20 passes through a 21 gauge (1.25 in. long) needle to achieve a cell lysate with a protein concentration between 0.5-4 mg mL-1. The homogenized sample was clarified by centrifugation at 20,000×g for 10 minutes at 4° C. A micro-BCA assay (Pierce) was used to determine the final protein concentration in the cell lysate. 200 μg protein for each sample were reduced and alkylated as previously described (An et al., 2017). Proteins were precipitated using methanol/chloroform. In brief, four volumes of methanol were added to the cell lysate, followed by one volume of chloroform, and finally three volumes of water. The mixture was vortexed and centrifuged at 14,000×g for 5 minutes to separate the chloroform phase from the aqueous phase. The precipitated protein was washed with three volumes of methanol, centrifuged at 14,000×g for 5 minutes, and the resulting washed precipitated protein was allowed to air dry. Precipitated protein was resuspended in 4 M Urea, 50 mM HEPES pH 7.4, followed by dilution to 1 M urea with the addition of 200 mM EPPS pH 8 for digestion with LysC (1:50; enzyme:protein) for 12 hours at room temperature. The LysC digestion was diluted to 0.5 M Urea, 200 mM EPPS pH 8 and then digested with trypsin (1:50; enzyme:protein) for 6 hours at 37′C. Tandem mass tag (TMT) reagents (Thermo Fisher Scientific) were dissolved in anhydrous acetonitrile (ACN) according to manufacturer's instructions. Anhydrous ACN was added to each peptide sample to a final concentration of 30% v/v, and labeling was induced with the addition of TMT reagent to each sample at a ratio of 1:4 peptide:TMT label. The 10-plex labeling reactions were performed for 1.5 hours at room temperature and the reaction quenched by the addition of 0.3% hydroxylamine for 15 minutes at room temperature. The sample channels were combined at a 1:1:1:1:1:1:1:1:1:1 ratio, desalted using C₁₈ solid phase extraction cartridges (Waters) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak, Waters). Samples were then offline fractionated into 96 fractions by high pH reverse-phase HPLC (Agilent LC1260) through an aeris peptide xb-c18 column (phenomenex) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 fractions or 48 fractions and every fraction was used for subsequent mass spectrometry analysis.

Data were collected using an Orbitrap Fusion Lumos mass spectrometer (Thermo Fisher Scientific, San Jose, CA, USA) coupled with a Proxeon EASY-nLC 1200 LC pump (Thermo Fisher Scientific). Peptides were separated on a 50 cm and 75 μm inner diameter Easyspray column (ES803, Thermo Fisher Scientific). Peptides were separated using a 3 hour gradient of 6-27% acetonitrile in 1.0% formic acid with a flow rate of 300 nL/min.

Each analysis used an MS3-based TMT method as described previously (McAlister et al., 2014). The data were acquired using a mass range of m/z 350-1350, resolution 120,000, AGC target 1×10⁶, maximum injection time 100 ms, dynamic exclusion of 90 seconds for the peptide measurements in the Orbitrap. Data dependent MS2 spectra were acquired in the ion trap with a normalized collision energy (NCE) set at 35%, AGC target set to 1.8×10⁴ and a maximum injection time of 120 ms. MS3 scans were acquired in the Orbitrap with a HCD collision energy set to 55%, AGC target set to 1.5×10⁵, maximum injection time of 150 ms, resolution at 50,000 and with a maximum synchronous precursor selection (SPS) precursors set to 10.

### LC-MS Data Analysis

Proteome Discoverer 2.2 (Thermo Fisher) was used for .RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 20 ppm, fragment ion mass tolerance of 0.6 Da, static alkylation of cysteine (57.02146 Da), static TMT labeling of lysine residues and N-termini of peptides (229.16293 Da), and variable oxidation of methionine (15.99491 Da). TMT reporter ion intensities were measured using a 0.003 Da window around the theoretical m/z for each reporter ion in the MS3 scan. Peptide spectral matches with poor quality MS3 spectra were excluded from quantitation (summed signal-to-noise across 10 channels >200 and precursor isolation specificity <0.5). Reporter ion intensities were normalized and scaled using in house scripts and the R framework (Team, 2013). Statistical analysis was carried out using the limma package within the R framework (Ritchie et al., 2015).

### CRISPR/Cas9 Mediated Genome Editing

For the generation of HEK293T^{CRBN-/-} and Kelly^{CRBN-/-} cells, HEK293T or Kelly cells were transfected with 4 μg of spCas9-sgRNA-mCherry using Lipofectamine 2000. 48 hours post transfection, pools of mCherry expressing cells were obtained by fluorescence assisted cell sorting (FACS). Two independent pools were sorted to avoid clonal effects and artifacts specific to a single pool. For SALL4 antibody validation, HEK293T or Kelly cells were transfected with 4 μg of spCas9-sgRNA-mCherry using Lipofectamine 2000. Protein levels were assessed by western blot 48 hours post-transfection.

guide RNA sequences used:

(SEQ ID NO: 19)
CRBN: TGCGGGTAAACAGACATGGC
(SEQ ID NO: 20)
SALL4-1: CCTCCTCCGAGTTGATGTGC
(SEQ ID NO: 21)
SALL4-2: ACCCCAGCACATCAACTCGG
(SEQ ID NO: 22)
SALL4-3: CCAGCACATCAACTCGGAGG

### REFERENCES

Afifi, H. H., Abdel-Salam, G. M., Eid, M. M., Tosson, A. M., Shousha, W. G., Abdel Azeem, A. A., Farag, M. K., Mehrez, M. I., and Gaber, K. R. (2016). Expanding the mutation and clinical spectrum of Roberts syndrome. Congenit Anom (Kyoto) 56, 154-162.
An, J., Ponthier, C. M., Sack, R., Seebacher, J., Stadler, M. B., Donovan, K. A., and Fischer, E. S. (2017). pSILAC mass spectrometry reveals ZFP91 as IMiD-dependent substrate of the CRL4CRBN ubiquitin ligase. Nat Commun 8, 15398.
Bai, N., Cui, X. Y., Wang, J., Sun, C. G., Mei, H. K., Liang, B. B., Cai, Y., Song, X. J., Gu, J. K., and Wang, R. (2013). Determination of thalidomide concentration in human plasma by liquid chromatography-tandem mass spectrometry. Exp Ther Med 5, 626-630.
Butler, H. (1977). The effect of thalidomide on a prosimian: the greater galago (Galago crassicaudatus). J Med Primatol 6, 319-324.
Cavadini, S., Fischer, E. S., Bunker, R. D., Potenza, A., Lingaraju, G. M., Goldie, K. N., Mohamed, W. I., Faty, M., Petzold, G., Beckwith, R. E., et al. (2016). Cullin-RING ubiquitin E3 ligase regulation by the COP9 signalosome. Nature 531, 598-603.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Chen, N., Zhou, S., and Palmisano, M. (2017). Clinical Pharmacokinetics and Pharmacodynamics of Lenalidomide. Clin Pharmacokinet 56, 139-152.
D'Amato, R. J., Loughnan, M. S., Flynn, E., and Folkman, J. (1994). Thalidomide is an inhibitor of angiogenesis. Proceedings of the National Academy of Sciences of the United States of America 91, 4082-4085.
Dahut, W. L., Aragon-Ching, J. B., Woo, S., Tohnya, T. M., Gulley, J. L., Arlen, P. M., Wright, J. J., Ventiz, J., and Figg, W. D. (2009). Phase I study of oral lenalidomide in patients with refractory metastatic cancer. J Clin Pharmacol 49, 650-660.
Eichner, R., Heider, M., Fernandez-Saiz, V., van Bebber, F., Garz, A. K., Lemeer, S., Rudelius, M., Targosz, B. S., Jacobs, L., Knom, A. M., et al. (2016). Immunomodulatory drugs disrupt the cereblon-CD147-MCT1 axis to exert antitumor activity and teratogenicity. Nat Med 22, 735-743.
Fischer, E. S., Bdhm, K., Lydeard, J. R., Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Gandhi, A. K., Kang, J., Havens, C. G., Conklin, T., Ning, Y., Wu, L., Ito, T., Ando, H., Waldman, M. F., Thakurta, A., et al. (2014a). Immunomodulatory agents lenalidomide and pomalidomide co-stimulate T cells by inducing degradation of T cell repressors Ikaros and Aiolos via modulation of the E3 ubiquitin ligase complex CRL4(CRBN). British journal of haematology 164, 811-821.
Gandhi, A. K., Mendy, D., Waldman, M., Chen, G., Rychak, E., Miller, K., Gaidarova, S., Ren, Y., Wang, M., Breider, M., et al. (2014b). Measuring cereblon as a biomarker of response or resistance to lenalidomide and pomalidomide requires use of standardized reagents and understanding of gene complexity. British journal of haematology 164, 233-244.
Heath, R. J., Goel, G., Baxt, L. A., Rush, J. S., Mohanan, V., Paulus, G. L. C., Jani, V., Lassen, K. G., and Xavier, R. J. (2016). RNF166 Determines Recruitment of Adaptor Proteins during Antibacterial Autophagy. Cell Rep 17, 2183-2194.
Heger, W., Klug, S., Schmahl, H. J., Nau, H., Merker, H. J., and Neubert, D. (1988). Embryotoxic effects of thalidomide derivatives on the non-human primate Callithrix jacchus; 3. Teratogenic potency of the EM 12 enantiomers. Archives of toxicology 62, 205-208.
Hoffmann, M., Kasserra, C., Reyes, J., Schafer, P., Kosek, J., Capone, L., Parton, A., Kim-Kang, H., Surapaneni, S., and Kumar, G. (2013). Absorption, metabolism and excretion of [14C]pomalidomide in humans following oral administration. Cancer Chemother Pharmacol 71, 489-501.
Ingalls, T. H., Curley, F. J., and Zappasodi, P. (1964). Thalidomide Embryopathy in Hybrid Rabbits. N Engl J Med 271, 441-444.
Ito, T., Ando, H., Suzuki, T., Ogura, T., Hotta, K., Imamura, Y., Yamaguchi, Y., and Handa, H. (2010). Identification of a primary target of thalidomide teratogenicity. Science (New York, NY) 327, 1345-1350.
Knobloch, J., and Ruther, U. (2008). Shedding light on an old mystery: thalidomide suppresses survival pathways to induce limb defects. Cell cycle (Georgetown, Tex) 7, 1121-1127.
Kohlhase, J. (1993). SALL4-Related Disorders. In GeneReviews®, M. P. Adam, H. H. Ardinger, R. A. Pagon, S. E. Wallace, L. J. H. Bean, H. C. Mefford, K. Stephens, A. Amemiya, and N. Ledbetter, eds. (Seattle (WA)).
Kohlhase, J. (2004). SALL4-Related Disorders. In GeneReviews, M. P. Adam, H. H. Ardinger, and R. A. Pagon, eds. (Seattle (WA): University of Seattle, Seattle).
Kohlhase, J., Schubert, L., Liebers, M., Rauch, A., Becker, K., Mohammed, S. N., Newbury-Ecob, R., and Reardon, W. (2003). Mutations at the SALL4 locus on chromosome 20 result in a range of clinically overlapping phenotypes, including Okihiro syndrome, Holt-Oram syndrome, acro-renal-ocular syndrome, and patients previously reported to represent thalidomide embryopathy. J Med Genet 40, 473-478.
Koshiba-Takeuchi, K., Takeuchi, J. K., Arruda, E. P., Kathiriya, I. S., Mo, R., Hui, C. C., Srivastava, D., and Bruneau, B. G. (2006). Cooperative and antagonistic interactions between Sa114 and Tbx5 pattern the mouse limb and heart. Nat Genet 38, 175-183.
Kronke, J., Fink, E. C., Hollenbach, P. W., MacBeth, K. J., Hurst, S. N., Udeshi, N. D., Chamberlain, P. P., Mani, D. R., Man, H. W., Gandhi, A. K., et al. (2015). Lenalidomide induces ubiquitination and degradation of CKlalpha in del(5q) MDS. Nature 523, 183-188.
Kronke, J., Udeshi, N. D., Narla, A., Grauman, P., Hurst, S. N., McConkey, M., Svinkina, T., Heckl, D., Comer, E., Li, X., et al. (2014). Lenalidomide causes selective degradation of IKZF1 and IKZF3 in multiple myeloma cells. Science 343, 301-305.
Lee, K. M., Yang, S. J., Kim, Y. D., Choi, Y. D., Nam, J. H., Choi, C. S., Choi, H. S., and Park, C. S. (2013). Disruption of the cereblon gene enhances hepatic AMPK activity and prevents high-fat diet-induced obesity and insulin resistance in mice. Diabetes 62, 1855-1864.
Lenz, W. (1962). Thalidomide and congenital abnormalities. The Journal of American Medical Association.
Lenz, W. (1988). A short history of thalidomide embryopathy. Teratology 38, 203-215.
Lu, G., Middleton, R. E., Sun, H., Naniong, M., Ott, C. J., Mitsiades, C. S., Wong, K. K., Bradner, J. E., and Kaelin, W. G., Jr. (2014). The myeloma drug lenalidomide promotes the cereblon-dependent destruction of Ikaros proteins. Science 343, 305-309.
Matyskiela, M. E., Lu, G., Ito, T., Pagarigan, B., Lu, C. C., Miller, K., Fang, W., Wang, N. Y., Nguyen, D., Houston, J., et al. (2016). A novel cereblon modulator recruits GSPT1 to the CRL4(CRBN) ubiquitin ligase. Nature 535, 252-257.
McAlister, G. C., Nusinow, D. P., Jedrychowski, M. P., Wuhr, M., Huttlin, E. L., Erickson, B. K., Rad, R., Haas, W., and Gygi, S. P. (2014). MultiNotch MS3 enables accurate, sensitive, and multiplexed detection of differential expression across cancer cell line proteomes. Anal Chem 86, 7150-7158.
McBride, W. G. (1961). Thalidomide and congenital abnormalities. The Journal of American Medical Association 2.
Najafabadi, H. S., Mnaimneh, S., Schmitges, F. W., Garton, M., Lam, K. N., Yang, A., Albu, M., Weirauch, M. T., Radovani, E., Kim, P. M., et al. (2015). C2H2 zinc finger proteins greatly expand the human regulatory lexicon. Nat Biotechnol 33, 555-562.
Neubert, D., Heger, W., Merker, H. J., Sames, K., and Meister, R. (1988). Embryotoxic effects of thalidomide derivatives in the non-human primate Callithrix jacchus. II. Elucidation of the susceptible period and of the variability of embryonic stages. Archives of toxicology 61, 180-191.
Nguyen, T. V., Lee, J. E., Sweredoski, M. J., Yang, S. J., Jeon, S. J., Harrison, J. S., Yim, J. H., Lee, S. G., Handa, H., Kuhlman, B., et al. (2016). Glutamine Triggers Acetylation-Dependent Degradation of Glutamine Synthetase via the Thalidomide Receptor Cereblon. Mol Cell 61, 809-820.
Nishihara, M., Yamada, M., Nozaki, M., Nakahira, K., and Yanagihara, I. (2010). Transcriptional regulation of the human establishment of cohesion 1 homolog 2 gene. Biochem Biophys Res Commun 393, 111-117.
Nowak, R. P., DeAngelo, S. L., Buckley, D., He, Z., Donovan, K. A., An, J., Safaee, N., Jedrychowski, M. P., Ponthier, C. M., Ishoey, M., et al. (2018). Plasticity in binding confers selectivity in ligand-induced protein degradation. Nat Chem Biol.
Pan, B., and Lentzsch, S. (2012). The application and biology of immunomodulatory drugs (IMiDs) in cancer. Pharmacol Ther 136, 56-68.
Patel, N., Shamseldin, H. E., Sakati, N., Khan, A. O., Softa, A., Al-Fadhli, F. M., Hashem, M., Abdulwahab, F. M., Alshidi, T., Alomar, R., et al. (2017). GZF1 Mutations Expand the Genetic Heterogeneity of Larsen Syndrome. Am J Hum Genet 100, 831-836.
Petzold, G., Fischer, E. S., and Thoma, N. H. (2016). Structural basis of lenalidomide-induced CK1alpha degradation by the CRL4 ubiquitin ligase. Nature 532, 127-130.
Raina, K., and Crews, C. M. (2017). Targeted protein knockdown using small molecule degraders. Curr Opin Chem Biol 39, 46-53.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Sakaki-Yumoto, M., Kobayashi, C., Sato, A., Fujimura, S., Matsumoto, Y., Takasato, M., Kodama, T., Aburatani, H., Asashima, M., Yoshida, N., et al. (2006). The murine homolog of SALL4, a causative gene in Okihiro syndrome, is essential for embryonic stem cell proliferation, and cooperates with Sall1 in anorectal, heart, brain and kidney development. Development 133, 3005-3013.
Schmitges, F. W., Radovani, E., Najafabadi, H. S., Barazandeh, M., Campitelli, L. F., Yin, Y., Jolma, A., Zhong, G., Guo, H., Kanagalingam, T., et al. (2016). Multiparameter functional diversity of human C₂H₂ zinc finger proteins. Genome Res 26, 1742-1752.
Sheereen, A., Alaamery, M., Bawazeer, S., Al Yafee, Y., Massadeh, S., and Eyaid, W. (2017). A missense mutation in the CRBN gene that segregates with intellectual disability and self-mutilating behaviour in a consanguineous Saudi family. J Med Genet 54, 236-240.
Smith, R. L., Fabro, S., Schumacher, H., and Williams (1965). Studies on the relationship between the chemical structure and embryotoxic activity of thalidomide and related compounds. In Embryopathic Activity of Drugs, Biological Council Symposium, J. M. Robson, F. Sullivan, and R. L. Smith, eds. (London: Churchill), pp. 194-209.
Team, R. C. (2013). R: A language and environment for statistical computing. R Foundation for Statistical Computing, Vienna, Austria.
Teo, S. K., Colburn, W. A., Tracewell, W. G., Kook, K. A., Stirling, D. I., Jaworsky, M. S., Scheffler, M. A., Thomas, S. D., and Laskin, O. L. (2004). Clinical pharmacokinetics of thalidomide. Clin Pharmacokinet 43, 311-327.
Vargesson, N. (2015). Thalidomide-induced teratogenesis: history and mechanisms. Birth Defects Res C Embryo Today 105, 140-156.
Vickers, T. H. (1967). The thalidomide embryopathy in hybrid rabbits. Br J Exp Pathol 48, 107-117.
Zakeri, B., Fierer, J. O., Celik, E., Chittock, E. C., Schwarz-Linek, U., Moy, V. T., and Howarth, M. (2012). Peptide tag forming a rapid covalent bond to a protein, through engineering a bacterial adhesin. Proc Natl Acad Sci USA 109, E690-697.

### Example 2: Identification of Compounds that do not Induce Degradation of SALL4

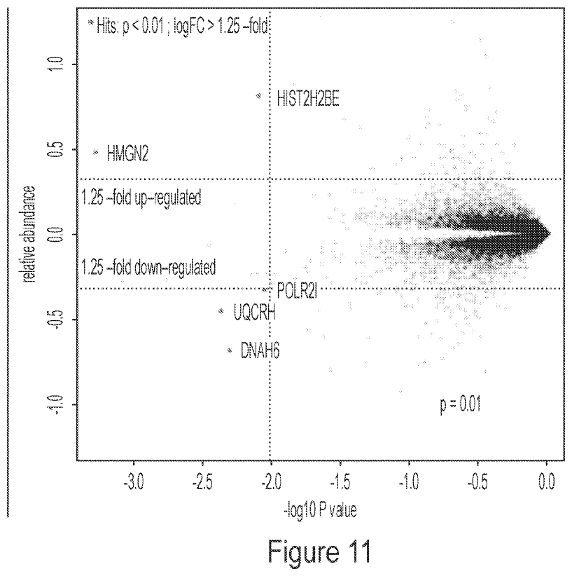

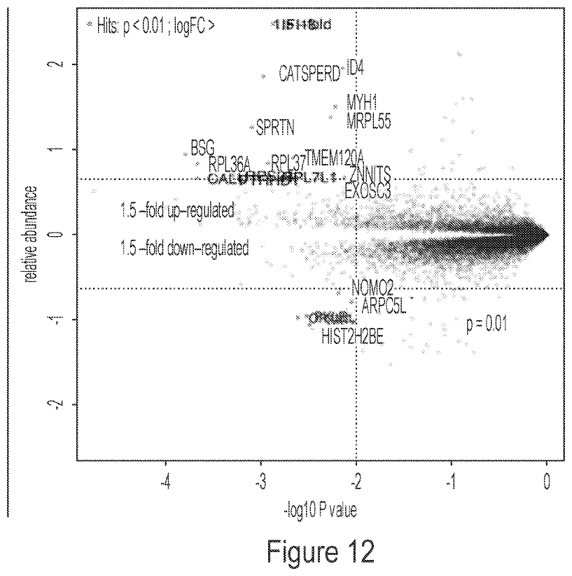

A library of approximately 100 IMiD compounds was generated and screened for the ability to degrade SALL4. Briefly, cells were treated with the library of IMiD compounds, and LC-MS was performed. The samples were prepared and the LC-MS data was analyzed as described in Example 1. Two compounds (DFCI1-DFCI2) were identified in which SALL4 degradation was not observed by LC-MS, indicating that the IMiD compounds are not teratogenic. This is shown in exemplary protein abundance data generated by LC-MS for compounds DFCI1 and DFCI2 shown in FIGS. 11 and 12, respectively.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1.-54. (canceled)

55. A transformed cell comprising:
a nucleic acid encoding SALL4; wherein if the nucleic acid is native to the cell, the nucleic acid is fused to a detectable label, and if the nucleic acid is recombinant to the cell, it may encode SALL4 or a fusion between SALL4 and the detectable label.

56. The transformed cell of claim 55, wherein SALL4 is human SALL4.

57. The transformed cell of claim 55, wherein SALL4 is native SALL4.

58. The transformed cell of claim 55, wherein SALL4 is recombinant.

59. The transformed cell of claim 55, wherein SALL4 comprises the nucleic acid sequence of SEQ ID NO: 1, or a sequence with 95% identity thereto.

60. The transformed cell of claim 55, wherein SALL4 is a SALL4 fragment that comprises or consists of an amino acid sequence of residues 370-440, 378-438, 410-433, or 402-436 of SEQ ID NO. 1, or a sequence with 95% identity thereto.

61. The transformed cell of claim 55, wherein SALL4 is a SALL4 fragment that comprises or consists of an amino acid sequence of residues 550-650, 594-616, 583-617, or 590-618 of SEQ ID NO. 1, or a sequence with 95% identity thereto.

62. The transformed cell of claim 55, wherein the detectable label is a biotin tag, a streptavidin tag, a spycatcher tag, a GST tag, a His-tag, a myc-tag, an S-peptide tag, a MBP tag, a GST tag, a FLAG tag, a thioredoxin tag, a GFP tag, a CFP tag, an RFP tag, a YFP tag, a BCCP, a calmodulin tag, a Strep tag, an HSV-epitope tag, a V5-epitope tag, a CBP tag or a component of a nanoBiT system.

63. The transformed cell of claim 62, wherein the component of the nanoBiT system is HiBiT, LoBiT, LgBiT, or SmBiT.

64. The transformed cell of claim 55, wherein the cell is a murine cell, a rat cell, a rabbit cell, a monkey cell, a zebrafish cell, or a human cell.


SEQUENCE LISTING (75 sequences)

SEQ ID NO: 1 - protein, 1053 aa, Homo sapiens
MSRRKQAKPQHINSEEDQGEQQPQQQTPEFADAAPAAPAAGELGAPVNHPGNDEVASEDEATVKRLRREETHVCEKCCAEFFSISEFLEHKKNCTKNPPVLIMNDSEGPVPSEDFSGAVLSHQPTSPGSKDCHRENGGSSEDMKEKPDAESVVYLKTETALPPTPQDISYLAKGKVANTNVTLQALRGTKVAVNQRSADALPAPVPGANSIPWVLEQILCLQQQQLQQIQLTEQIRIQVNMWASHALHSSGAGADTLKTLGSHMSQQVSAAVALLSQKAGSQGLSLDALKQAKLPHANIPSATSSLSPGLAPFTLKPDGTRVLPNVMSRLPSALLPQAPGSVLFQSPFSTVALDTSKKGKGKPPNISAVDVKPKDEAALYKHKCKYCSKVFGTDSSLQIHLRSHTGERPFVCSVCGHRFTTKGNLKVHFHRHPQVKANPQLFAEFQDKVAAGNGIPYALSVPDPIDEPSLSLDSKPVLVTTSVGLPQNLSSGTNPKDLTGGSLPGDLQPGPSPESEGGPTLPGVGPNYNSPRAGGFQGSGTPEPGSETLKLQQLVENIDKATTDPNECLICHRVLSCQSSLKMHYRTHTGERPFQCKICGRAFSTKGNLKTHLGVHRTNTSIKTQHSCPICQKKFTNAVMLQQHIRMHMGGQIPNTPLPENPCDFTGSEPMTVGENGSTGAICHDDVIESIDVEEVSSQEAPSSSSKVPTPLPSIHSASPTLGFAMMASLDAPGKVGPAPFNLQRQGSRENGSVESDGLTNDSSSLMGDQEYQSRSPDILETTSFQALSPANSQAESIKSKSPDAGSKAESSENSRTEMEGRSSLPSTFIRAPPTYVKVEVPGTFVGPSTLSPGMTPLLAAQPRRQAKQHGCTRCGKNFSSASALQIHERTHTGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKHQFPHFLEENKIAVS

SEQ ID NO: 2 - protein, 442 aa, Homo sapiens [sequence omitted]

SEQ ID NO: 3 - DNA, 22 bp, synthetic construct
  misc_feature 1..22: note = Synthetic Polynucleotide
  source 1..22: mol_type = other DNA
aagtactgtaacaaggtttttg

SEQ ID NO: 4 - DNA, 18 bp, synthetic construct
  misc_feature 1..18: note = Synthetic Polynucleotide
  source 1..18: mol_type = other DNA
acacttgtgcttgtagag

SEQ ID NO: 5 - DNA, 23 bp, synthetic construct
  misc_feature 1..23: note = Synthetic Polynucleotide
  source 1..23: mol_type = other DNA
tctgtctgtgctcatcgcttcac

SEQ ID NO: 6 - DNA, 18 bp, synthetic construct
  misc_feature 1..18: note = Synthetic Polynucleotide
  source 1..18: mol_type = other DNA
gcacacgaagggtctctc

SEQ ID NO: 7 - DNA, 27 bp, synthetic construct
  misc_feature 1..27: note = Synthetic Polynucleotide
  source 1..27: mol_type = other DNA
ctctgtctgtaatcatcgcttcaccac

SEQ ID NO: 8 - DNA, 18 bp, synthetic construct
  misc_feature 1..18: note = Synthetic Polynucleotide
  source 1..18: mol_type = other DNA
cacacgaagggtctctct

SEQ ID NO: 9 - DNA, 23 bp, synthetic construct
  misc_feature 1..23: note = Synthetic Polynucleotide
  source 1..23: mol_type = other DNA
aagatctgtgcccgagccttttc

SEQ ID NO: 10 - DNA, 18 bp, synthetic construct
  misc_feature 1..18: note = Synthetic Polynucleotide
  source 1..18: mol_type = other DNA
acactggaacggtctctc

SEQ ID NO: 11 - DNA, 27 bp, synthetic construct
  misc_feature 1..27: note = Synthetic Polynucleotide
  source 1..27: mol_type = other DNA
taagatctgtaaccgagccttttctac

SEQ ID NO: 12 - DNA, 18 bp, synthetic construct
  misc_feature 1..18: note = Synthetic Polynucleotide
  source 1..18: mol_type = other DNA
cactggaacggtctctcc

SEQ ID NO: 13 - DNA, 54 bp, synthetic construct
  misc_feature 1..54: note = Synthetic Polynucleotide
  source 1..54: mol_type = other DNA
agggcaatctcaaggtccactttcaccgacaccctcaggtgaaggcaaaccccc

SEQ ID NO: 14 - DNA, 55 bp, synthetic construct
  misc_feature 1..55: note = Synthetic Polynucleotide
  source 1..55: mol_type = other DNA
tggtggtgaagcggtgaccacagacagagcacacgaaaggtctctctccggtgtg

SEQ ID NO: 15 - DNA, 20 bp, synthetic construct
  misc_feature 1..20: note = Synthetic Polynucleotide
  source 1..20: mol_type = other DNA
ggtcctcgagcagatcttgt

SEQ ID NO: 16 - DNA, 20 bp, synthetic construct
  misc_feature 1..20: note = Synthetic Polynucleotide
  source 1..20: mol_type = other DNA
ggcatccagagacagacctt

SEQ ID NO: 17 - DNA, 19 bp, synthetic construct
  misc_feature 1..19: note = Synthetic Polynucleotide
  source 1..19: mol_type = other DNA
gaaggtgaaggtcggagtc

SEQ ID NO: 18 - DNA, 20 bp, synthetic construct
  misc_feature 1..20: note = Synthetic Polynucleotide
  source 1..20: mol_type = other DNA
gaagatggtgatgggatttc

SEQ ID NO: 19 - DNA, 20 bp, synthetic construct
  misc_feature 1..20: note = Synthetic Polynucleotide
  source 1..20: mol_type = other DNA
tgcgggtaaacagacatggc

SEQ ID NO: 20 - DNA, 20 bp, synthetic construct
  misc_feature 1..20: note = Synthetic Polynucleotide
  source 1..20: mol_type = other DNA
cctcctccgagttgatgtgc

SEQ ID NO: 21 - DNA, 20 bp, synthetic construct
  misc_feature 1..20: note = Synthetic Polynucleotide
  source 1..20: mol_type = other DNA
accccagcacatcaactcgg

SEQ ID NO: 22 - DNA, 20 bp, synthetic construct
  misc_feature 1..20: note = Synthetic Polynucleotide
  source 1..20: mol_type = other DNA
ccagcacatcaactcggagg

SEQ ID NO: 23 - protein, 23 aa, synthetic construct
  REGION 1..23: note = Synthetic Polypeptide
FQCNQCGASFTQKGNLLRHIKLH

SEQ ID NO: 24 - protein, 23 aa, synthetic construct
  REGION 1..23: note = Synthetic Polypeptide
LQCEICGFTCRQKASLNWHMKKH

SEQ ID NO: 25 - protein, 23 aa, synthetic construct
  REGION 1..23: note = Synthetic Polypeptide
FVCSVCGHRFTTKGNLKVHFHRH

SEQ ID NO: 26 - protein, 23 aa, synthetic construct
  REGION 1..23: note = Synthetic Polypeptide
FQCKICGRAFSTKGNLKTHLGVH

SEQ ID NO: 27 - protein, 33 aa, synthetic construct
  REGION 1..33: note = Synthetic Polypeptide
LNLIGRPSTEHSWFPGYAWTVAQCKICASHIGW

SEQ ID NO: 28 - protein, 33 aa, synthetic construct
  REGION 1..33: note = Synthetic Polypeptide
LNLIGRPSTEHSWFPGYAWTVAQCRICASHIGW

SEQ ID NO: 29 - protein, 33 aa, synthetic construct
  REGION 1..33: note = Synthetic Polypeptide
LNLIGRPSTVHSWFPGYAWTVAQCKICASHIGW

SEQ ID NO: 30 - protein, 33 aa, synthetic construct
  REGION 1..33: note = Synthetic Polypeptide
LNLSGRPSTEHSWFPGYAWTIAQCRICGNHMGW

SEQ ID NO: 31 - protein, 33 aa, synthetic construct
  REGION 1..33: note = Synthetic Polypeptide
LNLIGRPSTLHSWFPGYAWTIAQCRTCSSHMGW

SEQ ID NO: 32 - protein, 23 aa, synthetic construct
  REGION 1..23: note = Synthetic Polypeptide
YVCPICGHRFTTKGNLKVHLQRH

SEQ ID NO: 33 - protein, 23 aa, synthetic construct
  REGION 1..23: note = Synthetic Polypeptide
YVCPVCGHRFTTKGNLKVHFHRH

SEQ ID NO: 34 - protein, 23 aa, synthetic construct
  REGION 1..23: note = Synthetic Polypeptide
FKCNICGNRFTTKGNLKVHFQRH

SEQ ID NO: 35 - protein, 23 aa, synthetic construct
  REGION 1..23: note = Synthetic Polypeptide
YKCNICGNRFTTKGNLKVHFQRH

SEQ ID NO: 36 - protein, 23 aa, synthetic construct
  REGION 1..23: note = Synthetic Polypeptide
MNCDVCGLSCISFNVLMVHKRSH

SEQ ID NO: 37 - protein, 23 aa, synthetic construct
  REGION 1..23: note = Synthetic Polypeptide
LKCDICGIICIGPNVLMVHKRSH

SEQ ID NO: 38 - protein, 23 aa, synthetic construct
  REGION 1..23: note = Synthetic Polypeptide
YKCEFCGRSYKQRSSLEEHKERC

SEQ ID NO: 39 - protein, 24 aa, synthetic construct
  REGION 1..24: note = Synthetic Polypeptide
HKCGYCGRSYKQRSSLEEHKERCH

SEQ ID NO: 40 - protein, 23 aa, synthetic construct
  REGION 1..23: note = Synthetic Polypeptide
LQCEICGFTCRQKASLNWHQRKH

SEQ ID NO: 41 - protein, 23 aa, synthetic construct
  REGION 1..23: note = Synthetic Polypeptide
LQCEICGYQCRQRASLNWHMKKH

SEQ ID NO: 42 - protein, 24 aa, synthetic construct
  REGION 1..24: note = Synthetic Polypeptide
YRCDTCGQTFANRCNLKSHQRHVH

SEQ ID NO: 43 - protein, 23 aa, synthetic construct
  REGION 1..23: note = Synthetic Polypeptide
YRCTVCGHYSSTLNLMSKHVGVH

SEQ ID NO: 44 - protein, 23 aa, synthetic construct
  REGION 1..23: note = Synthetic Polypeptide
MRCDFCGAGFDTRAGLSSHARAH

SEQ ID NO: 45 - protein, 23 aa, synthetic construct
  REGION 1..23: note = Synthetic Polypeptide
TTCEVCGACFETRKGLSSHARSH

SEQ ID NO: 46 - protein, 23 aa, synthetic construct
  REGION 1..23: note = Synthetic Polypeptide
YSCHVCGFETELNVQFVSHMSLH

SEQ ID NO: 47 - protein, 23 aa, synthetic construct
  REGION 1..23: note = Synthetic Polypeptide
YSCKVCGKRFAHTSEFNYHRRIH

SEQ ID NO: 48 - protein, 23 aa, synthetic construct
  REGION 1..23: note = Synthetic Polypeptide
YGCTECGARFSQPSALKTHMRIH

SEQ ID NO: 49 - protein, 23 aa, synthetic construct
  REGION 1..23: note = Synthetic Polypeptide
FVCDECGARFTQNHMLIYHKRCH

SEQ ID NO: 50 - protein, 23 aa, synthetic construct
  REGION 1..23: note = Synthetic Polypeptide
YKCEECGKAFNRLSHLTTHKIIH

SEQ ID NO: 51 - protein, 23 aa, synthetic construct
  REGION 1..23: note = Synthetic Polypeptide
YKCEECGKAFSRLSHLTTHKRIH

SEQ ID NO: 52 - protein, 23 aa, synthetic construct
  REGION 1..23: note = Synthetic Polypeptide
YKCEECGKAFSQSSTLTTHKIIH

SEQ ID NO: 53 - protein, 23 aa, synthetic construct
  REGION 1..23: note = Synthetic Polypeptide
YKCEECGKAFKQSSTLTTHKRIH

SEQ ID NO: 54 - protein, 23 aa, synthetic construct
  REGION 1..23: note = Synthetic Polypeptide
YKCEECGKAFNLSSQLTTHKIIH

SEQ ID NO: 55 - protein, 23 aa, synthetic construct
  REGION 1..23: note = Synthetic Polypeptide
YKCEECGKAFNQSSHLTTHKMIH

SEQ ID NO: 56 - protein, 23 aa, synthetic construct
  REGION 1..23: note = Synthetic Polypeptide
YKCEECGKAFNQSANLTTHKRIH

SEQ ID NO: 57 - protein, 23 aa, synthetic construct
  REGION 1..23: note = Synthetic Polypeptide
YKCEECGKAFNQSSTLSKHKVIH

SEQ ID NO: 58 - protein, 23 aa, synthetic construct
  REGION 1..23: note = Synthetic Polypeptide
YKCEECGKAFNNSSILNRHKMIH

SEQ ID NO: 59 - protein, 23 aa, synthetic construct
  REGION 1..23: note = Synthetic Polypeptide
YKCKECGKAYNEASNLSTHKRIH

SEQ ID NO: 60 - protein, 23 aa, synthetic construct
  REGION 1..23: note = Synthetic Polypeptide
LACGECGWAFADPTALEQHRQLH

SEQ ID NO: 61 - protein, 23 aa, synthetic construct
  REGION 1..23: note = Synthetic Polypeptide
HRCGQCGKGLSSKTALRLHERTH

SEQ ID NO: 62 - protein, 23 aa, synthetic construct
  REGION 1..23: note = Synthetic Polypeptide
HGCTRCGKNFSSASALQIHERTH

SEQ ID NO: 63 - protein, 23 aa, synthetic construct
  REGION 1..23: note = Synthetic Polypeptide
YCCDQCGKQFTQLNALQRHRRIH

SEQ ID NO: 64 - protein, 23 aa, synthetic construct
  REGION 1..23: note = Synthetic Polypeptide
FHCNQCGASFTQKGNLLRHIKLH

SEQ ID NO: 65 - protein, 23 aa, synthetic construct
  REGION 1..23: note = Synthetic Polypeptide
FVCNICGRAFTTKGNLKVHYMTH

SEQ ID NO: 66 - protein, 24 aa, synthetic construct
  REGION 1..24: note = Synthetic Polypeptide
FTCDRCGKRFEKLDSVKFHTLKSH

SEQ ID NO: 67 - protein, 24 aa, synthetic construct
  REGION 1..24: note = Synthetic Polypeptide
FPCEFCGKRFEKPDSVAAHRSKSH

SEQ ID NO: 68 - protein, 24 aa, synthetic construct
  REGION 1..24: note = Synthetic Polypeptide
FSCNICGKKFEKKDSVVAHKAKSH

SEQ ID NO: 69 - protein, 24 aa, synthetic construct
  REGION 1..24: note = Synthetic Polypeptide
FPCELCGKKFKRKKDVKRHVLQVH

SEQ ID NO: 70 - protein, 23 aa, synthetic construct
  REGION 1..23: note = Synthetic Polypeptide
FMCNACGRTFTDKSTLRRHTSIH

SEQ ID NO: 71 - protein, 23 aa, synthetic construct
  REGION 1..23: note = Synthetic Polypeptide
FQCPICGLVIKRKSYWKRHMVIH

SEQ ID NO: 72 - protein, 23 aa, synthetic construct
  REGION 1..23: note = Synthetic Polypeptide
FPCDVCGKVFGRQQTLSRHLSLH

SEQ ID NO: 73 - protein, 23 aa, synthetic construct
  REGION 1..23: note = Synthetic Polypeptide
FMCETCGKSFASKEYLKHHNRIH

SEQ ID NO: 74 - protein, 23 aa, synthetic construct
  REGION 1..23: note = Synthetic Polypeptide
FTCETCGKSFKRKNHLEVHRRTH

SEQ ID NO: 75 - protein, 3435 aa, Homo sapiens
METSERTYRASNTYRVALVALTHRALAGLNLYSPROTHRALAVALASNGLYCYSVALTHRGLYHISPHETHRSERALAGLUASPLEUASNLEULEUILEALALYSASNTHRARGLEUGLUILETYRVALVALTHRALAGLUGLYLEUARGPROVALLYSGLUVALGLYMETTYRGLYLYSILEALAVALMETGLULEUPHEARGPROLYSGLYGLUSERLYSASPLEULEUPHEILELEUTHRALALYSTYRASNALACYSILELEUGLUTYRLYSGLNSERGLYGLUSERILEASPILEILETHRARGALAHISGLYASNVALGLNASPARGILEGLYARGPROSERGLUTHRGLYILEILEGLYILEILEASPPROGLUCYSARGMETILEGLYLEUARGLEUTYRASPGLYLEUPHELYSVALILEPROLEUASPARGASPASNLYSGLULEULYSALAPHEASNILEARGLEUGLUGLULEUHISVALILEASPVALLYSPHELEUTYRGLYCYSGLNALAPROTHRILECYSPHEVALTYRGLNASPPROGLNGLYARGHISVALLYSTHRTYRGLUVALSERLEUARGGLULYSGLUPHEASNLYSGLYPROTRPLYSGLNGLUASNVALGLUALAGLUALASERMETVALILEALAVALPROGLUPROPHEGLYGLYALAILEILEILEGLYGLNGLUSERILETHRTYRHISASNGLYASPLYSTYRLEUALAILEALAPROPROILEILELYSGLNSERTHRILEVALCYSHISASNARGVALASPPROASNGLYSERARGTYRLEULEUGLYASPMETGLUGLYARGLEUPHEMETLEULEULEUGLULYSGLUGLUGLNMETASPGLYTHRVALTHRLEULYSASPLEUARGVALGLULEULEUGLYGLUTHRSERILEALAGLUCYSLEUTHRTYRLEUASPASNGLYVALVALPHEVALGLYSERARGLEUGLYASPSERGLNLEUVALLYSLEUASNVALASPSERASNGLUGLNGLYSERTYRVALVALALAMETGLUTHRPHETHRASNLEUGLYPROILEVALASPMETCYSVALVALASPLEUGLUARGGLNGLYGLNGLYGLNLEUVALTHRCYSSERGLYALAPHELYSGLUGLYSERLEUARGILEILEARGASNGLYILEGLYILEHISGLUHISALASERILEASPLEUPROGLYILELYSGLYLEUTRPPROLEUARGSERASPPROASNARGGLUTHRASPASPTHRLEUVALLEUSERPHEVALGLYGLNTHRARGVALLEUMETLEUASNGLYGLUGLUVALGLUGLUTHRGLULEUMETGLYPHEVALASPASPGLNGLNTHRPHEPHECYSGLYASNVALALAHISGLNGLNLEUILEGLNILETHRSERALASERVALARGLEUVALSERGLNGLUPROLYSALALEUVALSERGLUTRPLYSGLUPROGLNALALYSASNILESERVALALASERCYSASNSERSERGLNVALVALVALALAVALGLYARGALALEUTYRTYRLEUGLNILEHISPROGLNGLULEUARGGLNILESERHISTHRGLUMETGLUHISGLUVALALACYSLEUASPILETHRPROLEUGLYASPSERASNGLYLEUSERPROLEUCYSALAILEGLYLEUTRPTHRASPILESERALAARGILELEULYSLEUPROSERPHEGLULEULEUHISLYSGLUMETLEUGLYGLYGLUILEILEPROARGSERILELEUMETTHRTHRPHEGLUSERSERHISTYRLEULEUCYSALALEUGLYASPGLYALALEUPHETYRPHEGLYLEUASNILEGLUTHRGLYLEULEUSERASPARGLYSLYSVALTHRLEUGLYTHRGLNPROTHRVALLEUARGTHRPHEARGSERLEUSERTHRTHRASNVALPHEALACYSSERASPARGPROTHRVALILETYRSERSERASNHISLYSLEUVALPHESERASNVALASNLEULYSGLUVALASNTYRMETCYSPROLEUASNSERASPGLYTYRPROASPSERLEUALALEUALAASNASNSERTHRLEUTHRILEGLYTHRILEASPGLUILEGLNLYSLEUHISILEARGTHRVALPROLEUTYRGLUSERPROARGLYSILECYSTYRGLNGLUVALSERGLNCYSPHEGLYVALLEUSERSERARGILEGLUVALGLNASPTHRSERGLYGLYTHRTHRALALEUARGPROSERALASERTHRGLNALALEUSERSERSERVALSERSERSERLYSLEUPHESERSERSERTHRALAPROHISGLUTHRSERPHEGLYGLUGLUVALGLUVALHISASNLEULEUILEILEASPGLNHISTHRPHEGLUVALLEUHISALAHISGLNPHELEUGLNASNGLUTYRALALEUSERLEUVALSERCYSLYSLEUGLYLYSASPPROASNTHRTYRPHEILEVALGLYTHRALAMETVALTYRPROGLUGLUALAGLUPROLYSGLNGLYARGILEVALVALPHEGLNTYRSERASPGLYLYSLEUGLNTHRVALALAGLULYSGLUVALLYSGLYALAVALTYRSERMETVALGLUPHEASNGLYLYSLEULEUALASERILEASNSERTHRVALARGLEUTYRGLUTRPTHRTHRGLULYSGLULEUARGTHRGLUCYSASNHISTYRASNASNILEMETALALEUTYRLEULYSTHRLYSGLYASPPHEILELEUVALGLYASPLEUMETARGSERVALLEULEULEUALATYRLYSPROMETGLUGLYASNPHEGLUGLUILEALAARGASPPHEASNPROASNTRPMETSERALAVALGLUILELEUASPASPASPASNPHELEUGLYALAGLUASNALAPHEASNLEUPHEVALCYSGLNLYSASPSERALAALATHRTHRASPGLUGLUARGGLNHISLEUGLNGLUVALGLYLEUPHEHISLEUGLYGLUPHEVALASNVALPHECYSHISGLYSERLEUVALMETGLNASNLEUGLYGLUTHRSERTHRPROTHRGLNGLYSERVALLEUPHEGLYTHRVALASNGLYMETILEGLYLEUVALTHRSERLEUSERGLUSERTRPTYRASNLEULEULEUASPMETGLNASNARGLEUASNLYSVALILELYSSERVALGLYLYSILEGLUHISSERPHETRPARGSERPHEHISTHRGLUARGLYSTHRGLUPROALATHRGLYPHEILEASPGLYASPLEUILEGLUSERPHELEUASPILESERARGPROLYSMETGLNGLUVALVALALAASNLEUGLNTYRASPASPGLYSERGLYMETLYSARGGLUALATHRALAASPASPLEUILELYSVALVALGLUGLULEUTHRARGILEHISSEQUENCELISTING